(19) United States
(12) Patent Application Publication
(10) Pub. No.: US 2020/0216435 A1
(43) Pub. Date: Jul. 9, 2020

(54) BICYCLIC HETEROAROMATIC AMIDE COMPOUNDS FOR USE IN THERAPY

(71) Applicant: European Molecular Biology Laboratory, Heidelberg (DE)

(72) Inventors: David William Will, Heidelberg (DE); George Reid, Heidelberg (DE); Iryna Charapitsa, Heidelberg (DE); Joe Lewis, Dielheim (DE)

(21) Appl. No.: 16/621,608

(22) PCT Filed: Jun. 14, 2018
(86) PCT No.: PCT/EP2018/065817

§ 371 Date: Dec. 11, 2019

(30) Foreign Application Priority Data
Jun. 14, 2017 (EP) 17175896.4

(51) Int. Cl. C07D 417/12; A61P 35/00

(52) U.S. Cl. CPC C07D 417/12; A61P 35/00

(57) ABSTRACT

The present invention relates to compounds of the formula I as described below or a tautomer or a pharmaceutically acceptable salt thereof; to a pharmaceutical composition containing such compounds; and to said compounds of the formula I or a tautomer or a pharmaceutically acceptable salt thereof for use as a medicament, especially for use in the treatment or prevention of a disease or disorder selected from the group consisting of an inflammatory disease, a hyperproliferative disease or disorder, a hypoxia-related pathology and a disease characterized by excessive vascularization, wherein X¹ is CR¹ or N; X² is CR² or N; X³ is CR³ or N; X⁴ is CR⁴ or N; Y¹ is N, NR^{5a}, S, O or CR^{5b}; Y2 is N, NR^{5c}, S, O or CR^{5d}; Z is N or C; with the proviso that at most two of X¹, X², X³ and X⁴ are N; with the proviso that Y¹ is not O if Y2 is CR5d and simultaneously Z is C; with the proviso that Y¹ and Y² are not both simultaneously O or S; with the proviso that at least one of Y¹, Y² and Z is a heteroatom or heteroatom-containing group; L¹ is a bond, optionally substituted C₁-C₆-alkylene or C₃-C₈-cycloalkylene; L² is a bond, optionally substituted C₁-C₆-alkylene, C₃-C₈-cycloalkylene etc.; A is 3-, 4-, 5-, 6-, 7- or 8-membered optionally substituted, saturated, partially unsaturated or maximally unsaturated carbocyclic or heterocyclic ring; or L²-A forms a group C₁-C₆-alkylene-OR¹³, C₁-C₆-alkylene-SR¹⁴ or C₁-C₆-alkylene-NR¹⁵SR¹⁶; and R¹, R², R³, R⁴, R^{5a}, R^{5b}, NR^{5c}, R^{5d}, R⁶, R¹³, R¹⁴, R¹⁵ and R¹⁶ are as defined in the claims and the description.

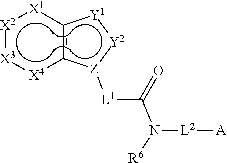

### CROSS-REFERENCE TO RELATED APPLICATION(S)

This patent application claims the benefit of priority of EP Application No. 17175896.4, filed Jun. 14, 2017.

### SEQUENCE LISTING

The instant application contains a Sequence Listing which has been submitted in ASCII format via EFS-Web and is hereby incorporated by reference in its entirety. Said ASCII copy, created on Feb. 21, 2020, is named 05710_039US1_SL.txt and is 1,154 bytes in size.

### FIELD OF THE INVENTION

The present invention relates to bicyclic heteroaromatic amide compounds, to a pharmaceutical composition containing these compounds, and to these compounds for use in therapy, especially for use in the treatment or prevention of a disease or disorder selected from the group consisting of an inflammatory disease, a hyperproliferative disease or disorder, a hypoxia-related pathology and a disease characterized by excessive vascularization.

### BACKGROUND OF THE INVENTION

Despite the recent extraordinary progress seen in cancer therapy using molecularly targeted drugs, cancer remains a major cause of death worldwide. The major barrier to successful treatment and prevention of cancer lies in the fact that many cancers are resistant or refractory to current chemotherapeutic and/or immunotherapy intervention, and many individuals suffer recurrence or death, even after aggressive therapy. Therefore, there is an ongoing need for expanding the treatment options for cancer patients, including the provision of new drugs.

Reductive characterization of tumors has uncovered a set of phenotypic states necessary for malignancy. These phenotypic states consist of distinct traits that are necessary and sufficient for malignancy. One of the earliest and most consistent traits of malignancy is the acquisition of a distinct metabolic programme, where cells limit their generation of energy largely to glycolytic fermentation, even when oxygen is available. This phenotype, known as aerobic glycolysis or the Warburg effect, was first reported by the Nobel laureate Otto Warburg in the 1930s' (O. Warburg et al., Berlin-Dahlem. London: Constable & Co. Ltd. (1930); O. Warburg, Science, 1956, 123, 309-314; 0. Warburg, Science, 1956, 124, 269-270) and differentiates proliferating cells from quiescent cells. Substrates for this aerobic glycolysis are glucose or amino acids, in particular glutamine or asparagine.

The PI3K-Akt-mTOR (phosphatidyl inositol 3 kinase, Akt Serine/Threonine Kinase and Mechanistic Target Of Rapamycin) cascade is a major signaling pathway that induces aerobic glycolysis and is associated with the development of the majority of cancers. The Akt signaling pathway is, thus, a major target for the development of cancer therapeutics (J. S. Brown et al., Pharmacol Ther., 2017, 172, 101-115).

The egr1 gene is an immediate early gene whose activity is controlled by expression. Its expression product, EGR1, is a transcription factor belonging to the family of Cys₂-His₂ zinc finger proteins. EGR1 is known to have a significant role in cancer (Baron et al., Cancer Gene Therapy, 2006, 13, 115-124). EGR1 integrates signals from many different pathways (I. Gudernova et al., Elife. 6:e21536 (2017)). EGR1 can act as tumor suppressor gene in fibrosarcoma, glioblastoma and in lung and breast cancer (C. Liu et al., J Biol Chem, 1999, 274(7), 4400-4411; C. Liu et al., J Biol Chem, 2000, 275(27), 20315-20323; M. M. Shareef et al., Cancer Res, 2007, 67(24), 11811-11820; R. P. Huang et al., Int J Cancer, 1997, 72(1), 102-109). EGR1 suppresses tumorogenesis by transactivating expression of TGFβ1, PTEN, fibronectin and p53 and by cooperating with Sp1, Jun-B and p21 (C. Liu et al., J Biol Chem, 1999, 274(7), 4400-4411; C. Liu et al., Cancer Gene Ther, 1998, 5(1), 3-28; V. Baron et al., Cancer Gene Ther, 2006, 13(2), 115-124). Therefore, compounds causing up-regulation of EGR1 expression at low dosage are considered to be useful in therapy of cancer and other proliferative diseases.

HSF1 (heat shock factor 1) is a transcription factor that is the master regulator of the expression of heat shock transcripts. C. Dai et al., Cell. 130:1005-18 (2007) found that HSF1 knock-out mice are resistant to chemically induced carcinogenesis and concluded that HSF1 is a central player in cancer. Moreover, HSF1 facilitates oncogenesis promoted by mutant p53. A large body of work has verified the importance of HSF1 in tumorigenesis and in cancer progression (see e.g. L. Whitesell et al., Expert Opin. Ther. Targets 2009, 13, 469-478; C. L. Moore, et al., ACS Chem. Biol. 2016, 11, 200-210, E. de Billy, et al., Oncotarget 2012, 3, 741-743). HSF1 supports the most aggressive forms of breast, lung and colon cancer, with HSF1-driven transcriptional programmes strongly associated with metastasis and death in a wide range of cancer (Mendillo et al., Cell 150: 549 (2012)). Finally, Kaplan Meier analysis demonstrates that patients whose tumors express high levels of HSF1 have a much poorer prognosis than patients expressing less HSF1, in multiple tumor types (B. Gyorffy et al. PLos One 8:e82241 (2013). C. Dai et al., Cell. 130:1005-18 (2007) further found that fibroblasts from HSF1 knockout mice have a lower requirement for glucose. Additionally, rohinitib, a rocaglamide that, amongst other activities (M. Li-Weber, Int J Cancer, 2015, 137(8), 1791-1799), prevents HSF1 binding to target enhancer elements, reduces glucose uptake of tumour cells (S. Santagata et al., Science, 2013, 341(6143):1238303). In conclusion, HSF1 has a sentinel, permissive role in licensing aerobic glycolysis by modulating glucose and neutral amino acid metabolism. Consequently, compromising HSF1 activity is an attractive target for new, effective and safe cancer treatment.

Pirin is a non-haem, iron containing protein that acts as a redox sensor in cells. It is ubiquitously expressed and is frequently expressed at higher levels in tumor cells than in surrounding normal tissue. For example, pirin has been linked to metastasis in myeloma (S. Licciulli et al., Am J Pathol, 2011, 178(5), 2397-2406; I. Miyazaki et al., Nat Chem Biol, 2010, 6(9), 667-673), is upregulated in the spleen and kidney of superoxide dismutase deficient mice (K. Brzoska et al., Redox Rep, 2011, 16(3), 129-133) and in the lungs of chronic smokers (B. D. Gelbman et al., Respir Res, 2007, 8:10). Pirin undergoes a conformational switch upon oxidation of the bound iron from Fe²⁺ to Fe³⁺. Oxidized pirin promotes the interaction of target promoters with the transcription factor NF-kB, a critical mediator of intracellular signaling that has been linked to cellular responses to proinflammatory signals and which controls the expression of a large array of genes involved in immune and stress responses (Lui et al., Proc. Natl. Acad. Sci. USA, 110:9722-7 (2013)).

M. D. Cheeseman et al., J Med Chem. 60:180-201 (2017) recently found that pirin is a key regulator of HSF1 and that small molecule ligands to pirin efficiently inhibit HSF1-mediated stress pathway. The authors could confirm in a human ovarian carcinoma xenograft model that their pirin ligand showed 70% tumor growth inhibition.

It is apparent from the foregoing that small molecule ligands to pirin will likely be useful in therapy of cancer and other proliferative diseases and also for therapy of inflammatory diseases, hypoxia-related pathologies and diseases characterized by excessive vascularization.

It is an object of the present invention to provide new therapeutic agents which allow for an efficient treatment of different proliferative and inflammatory diseases or disorders, hypoxia-related pathologies and/or diseases characterized by excessive vascularization. The compounds should be efficient ligands to pirin at low dosage and should cause up-regulation of EGR1 expression at low EC50 values. Expediently, the compounds should also downregulate the HSF1 expression and/or should also show good bioavailability and/or metabolic stability and/or low blockade of the hERG channel.

It was now found that the compounds of formula (I) as described herein efficiently cause up-regulation of EGR1 expression at low EC50 values, indicating that the compounds of formula (I) are efficient ligands to pirin.

### SUMMARY OF THE INVENTION

The present invention relates to compounds of the formula I as described below or a tautomer or a pharmaceutically acceptable salt thereof; to a pharmaceutical composition containing such compounds; and to the compounds of the formula I as described below or a tautomer or a pharmaceutically acceptable salt thereof for use as a medicament, especially for use in the treatment or prevention of a disease or disorder selected from the group consisting of an inflammatory disease, a hyperproliferative disease or disorder, a hypoxia-related pathology and a disease characterized by excessive vascularization.

Thus, in one aspect, the present invention relates to a compound of the formula I or a tautomer or a pharmaceutically acceptable salt thereof

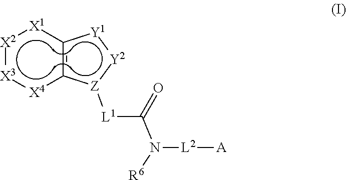

wherein

X¹ is CR¹ or N;

X² is CR² or N;

X³ is CR³ or N;

X⁴ is CR⁴ or N;

with the proviso that at most two of X¹, X², X³ and X⁴ are N;

Y¹ is N, NR^{5a}, S, O or CR^{5b};

Y² is N, NR^{5c}, S, O or CR^{5d};

Z is N or C;

with the proviso that Y¹ is not O if Y² is CR^{5d} and simultaneously Z is C;

with the proviso that Y¹ and Y² are not both simultaneously O or S;

with the proviso that at least one of Y¹, Y² and Z is a heteroatom or heteroatom-containing group;
L¹ is a bond, C₁-C₆-alkylene which may carry one or more substituents R⁷, or C₃-C₈-cycloalkylene which may carry one or more substituents R⁸;
L² is a bond, C₁-C₆-alkylene which may carry one or more substituents R⁷, C₃-C₈-cycloalkylene which may carry one or more substituents R⁸, C₁-C₆-alkylene-O, C₁-C₆-alkylene-S, C₁-C₆-alkylene-NR⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸;
A is 3-, 4-, 5-, 6-, 7- or 8-membered saturated, partially unsaturated or maximally unsaturated carbocyclic ring which may carry one or more substituents R⁹; or a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁰;
or L²-A forms a group C₁-C₆-alkylene-OR³, C₁-C₆-alkylene-SR¹⁴ or C₁-C₆-alkylene-NR¹⁵SR¹⁶;
R¹, R², R³ and R⁴, independently of each other, are selected from the group consisting of hydrogen, halogen, CN, nitro, SF₆, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹ and R², or R² and R³, or R³ and R⁴, together with the carbon atoms they are bound to, form a 3-, 4-, 5-, 6- or 7-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members, where the carbocyclic or heterocyclic ring may carry one or more substituents R¹⁸;
R^{5a}, R^{5b}, R^{5c} and R^{5d}, independently of each other, are selected from the group consisting of hydrogen, C₁-C₆-alkyl, C₁-C₆-haloalkyl, aryl, aryl-C₁-C₃-alkyl, where the aryl moiety in the two last-mentioned radicals may carry one or more substituents R¹⁸; hetaryl and hetaryl-C₁-C₃-alkyl, where hetaryl is a 5- or 6-membered heteroaromatic ring containing 1, 2, 3, or 4 heteroatoms selected from the group consisting of O, S and N as ring members, where the heteroaromatic ring may carry one or more substituents R¹⁸;
R⁶ is selected from the group consisting of hydrogen, C₁-C₆-alkyl which may carry one or more substituents R¹¹, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, where cycloalkyl in the two last-mentioned radicals may carry one or more substituents R¹²; C₁-C₆-alkoxy, C₁-C₆-haloalkoxy, aryl, aryl-C₁-C₃-alkyl, where the aryl moiety in the two last-mentioned radicals may carry one or more substituents R¹⁸; heterocyclyl and heterocyclyl-C₁-C₃-alkyl, where heterocyclyl is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R⁷ and R⁸, independently of each other and independently of each occurrence, are selected from the group consisting of F, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
   or two radicals R⁷ bound on the same carbon atom of the alkylene group, or two radicals R⁸ bound on the same carbon atom of the cycloalkylene group form together a group ═O or ═S;
each R⁹ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on adjacent ring atoms, together with the ring atoms they are bound to, may form a saturated, partially unsaturated or maximally unsaturated 3-, 4-, 5- or 6-membered carbocyclic ring which may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on non-adjacent ring atoms may form a bridge —CH₂— or —(CH₂)₂—;
each R¹⁰ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R¹⁰ bound on adjacent ring atoms, together with the ring atoms they are bound to, may form a saturated, partially unsaturated or maximally unsaturated 3-, 4-, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹¹ is independently selected from the group consisting of CN, nitro, SF₅, C₃-C₈-cycloalkyl which may carry one or more substituents R¹², OR¹³, S(O)R¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹² is independently selected from the group consisting of halogen, CN, nitro, SF₅, C₁-C₆-alkyl, C₁-C₆-haloalkyl, C₃-C₈-cycloalkyl, C₃-C₈-halocycloalkyl, OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹³ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, S(O)ₘR¹⁴, C(O)R¹⁷, C(O)OR²¹, C(O)NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁴ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, S(O)ₘR²², C(O)R¹⁷, C(O)OR²¹, C(O)NR²³R²⁴, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁸ is independently selected from the group consisting of halogen, CN, nitro, OH, SH, SF₅, C₁-C₆-alkyl which may carry one or more substituents selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; C₁-C₆-haloalkyl, C₃-C₈-cycloalkyl which may carry one or more substituents selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl; C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl, C₁-C₆-haloalkylcarbonyl, C₁-C₆-alkoxycarbonyl, C₁-C₆-haloalkoxycarbonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
or two radicals R¹⁸ bound on adjacent ring atoms, together with the ring atoms they are bound to, may form a saturated, partially unsaturated or maximally unsaturated 3-, 4-, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₃-C₈-cycloalkyl, C₃-C₈-halocycloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
each R²⁰ is independently selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl;
R²¹ and R²², independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl, C₃-C₈-halocycloalkyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;

m is 1 or 2; and

n is 0, 1 or 2.

Y¹, Y² and Z combine in such a way that the resulting condensed ring system containing X¹ to X⁴ and Y¹, Y² and Z as ring members is heteroaromatic.

The proviso that at least one of Y¹, Y² and Z is a heteroatom or heteroatom-containing group can be expressed alternatively in that Y¹, Y² and Z cannot be simultaneously a carbon ring atom (group); i.e. Y¹ cannot be CR^{5b} if Y² is CR^{5d} and simultaneously Z is C; Y² cannot be CR^{5d} if Y¹ is CR^{5b} and simultaneously Z is C; and Z cannot be C if Y¹ is CR^{5b} and simultaneously Y² is CR^{5d}.

In another aspect, the invention relates to a pharmaceutical composition containing a compound of formula I or a tautomer or a pharmaceutically acceptable salt thereof for use as a medicament. The composition may contain one or more than one compound I.

In another aspect, the invention relates to a compound of formula I or a tautomer or a pharmaceutically acceptable salt thereof for use as a medicament.

In another aspect, the invention relates to a compound of formula I or a tautomer or a pharmaceutically acceptable salt thereof for use in the treatment of conditions, disorders or diseases selected from the group consisting of inflammatory diseases, hyperproliferative diseases or disorders, a hypoxia related pathology and a disease characterized by pathophysiological hypervascularization.

In yet another aspect, the invention relates to the use of a compound of formula I or a tautomer or a pharmaceutically acceptable salt thereof for preparing a medicament for the treatment of conditions, disorders or diseases selected from the group consisting of inflammatory diseases, hyperproliferative diseases or disorders, a hypoxia related pathology and a disease characterized by pathophysiological hypervascularization.

In yet another aspect, the invention relates to a method for treating conditions, disorders or diseases selected from the group consisting of inflammatory diseases, hyperproliferative diseases or disorders, a hypoxia related pathology and a disease characterized by pathophysiological hypervascularization, which method comprises administering to a subject in need thereof a compound of formula I or a tautomer or a pharmaceutically acceptable salt thereof or a pharmaceutical composition containing a compound of formula I or a tautomer or a pharmaceutically acceptable salt thereof.

### DETAILED DESCRIPTION OF THE INVENTION

Provided the compounds of the formula I of a given constitution may exist in different spatial arrangements, for example if they possess one or more centers of asymmetry, polysubstituted rings or double bonds, or as different tautomers, the invention also relates to enantiomeric mixtures, in particular racemates, diastereomeric mixtures and tautomeric mixtures, preferably, however, the respective essentially pure enantiomers (enantiomerically pure), diastereomers and tautomers of the compounds of formula (I) and/or of their salts.

One center of asymmetry is for example L¹ if this is methylene substituted by one R⁷ or by two different R⁷, or is C₂-C₆-alkylene with at least one asymmetric C atom, or is C₃-C₈-cycloalkylene with at least one asymmetric C atom. One example for such L¹ being a center of asymmetry is CH(CH₃). Analogously, L² can be a center of asymmetry if this is methylene substituted by one R⁷ or by two different R⁷, or is C₂-C₆-alkylene with at least one asymmetric C atom, or is C₃-C₈-cycloalkylene with at least one asymmetric C atom. Other centers of chirality are for example compounds I in which A is saturated or partially unsaturated carbocyclic or heterocyclic ring containing at least one asymmetric C atom.

Racemates obtained can be resolved into the isomers mechanically or chemically by methods known per se. Diastereomers are preferably formed from the racemic mixture by reaction with an optically active resolving agent. Examples of suitable resolving agents are optically active acids, such as the D and L forms of tartaric acid, diacetyltartaric acid, dibenzoyltartaric acid, mandelic acid, malic acid, lactic acid or the various optically active camphorsulfonic acids, such as D- or L-camphorsulfonic acid. Also advantageous is enantiomer resolution with the aid of a column filled with an optically active resolving agent (for example dinitrobenzoylphenylglycine); an example of a suitable eluent is a hexane/isopropanol/acetonitrile mixture. The diastereomer resolution can also be carried out by standard purification processes, such as, for example, chromatography or fractional crystallization. It is also possible to obtain optically active compounds of formula (I) by the methods described below by using starting materials which are already optically active.

The invention also relates to “pharmaceutically acceptable salts” of the compounds of the formula (I), especially acid addition salts with physiologically tolerated, i.e. pharmaceutically acceptable acids. Examples of suitable physiologically tolerated organic and inorganic acids include, but are not limited to, hydrochloric acid, hydrobromic acid, phosphoric acid, sulfuric acid, C₁-C₄-alkylsulfonic acids, such as methanesulfonic acid, aromatic sulfonic acids, such as benzenesulfonic acid and toluenesulfonic acid, carboxylic acids such as oxalic acid, malic acid, maleic acid, fumaric acid, lactic acid, tartaric acid, adipic acid, mandelic acid, salicylic acid, phenylpropionic acid, nicotinic acid, benzoic acid acetate, alginic acid, ascorbic acid, aspartic acid, tannic acid, butyric acid, camphoric acid, citric acid, clavulanic acid, cyclopentanepropionic acid, gluconic acid, formic acid, acetic acid, propionic acid, pivalic acid, valeric acid, hexoic acid, heptoic acid, oleic acid, palmitic acid, pantothenic acid, pectinic acid, stearic acid, hexylresorcinic acid, hydroxynaphthoic acid, lactobionic acid and mucic acid. Other utilizable acids are described in Fortschritte der Arzneimittelforschung [Advances in drug research], Volume 10, pages 224 ff., Birkhauser Verlag, Basel and Stuttgart, 1966 and in Berge, S. M., et al., “Pharmaceutical Salts”, Journal of Pharmaceutical Science, 1977, 66, 1-19. Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formiate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like. Certain specific compounds of the present invention contain both basic and acidic functionalities that allow the compounds to be converted into either base or acid addition salts. Furthermore, where the compound of the invention carries an acidic moiety, suitable pharmaceutically acceptable salts thereof may include alkali metal salts (e.g., sodium or potassium salts); alkaline earth metal salts (e.g., calcium or magnesium salts); and salts formed with suitable organic ligands (e.g., ammonium, quaternary ammonium and amine cations formed using counteranions such as halide, hydroxide, carboxylate, sulfate, phosphate, nitrate, alkyl sulfonate and aryl sulfonate).

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

The invention also relates to N-oxides of the compounds of the formula (I), provided that those compounds contain a basic nitrogen atom, such as the nitrogen atom of a nitrogen containing heterocycle which may be present A, or one of X¹ to X⁴ being N. Examples of nitrogen containing heterocycle, where the nitrogen may be present in the form of an N-oxide, include pyridinyl, pyrimidinyl, pyrazinyl, pyridazinyl, pyrazolyl, imidazolyl, oxazolyl, oxadiazolyl, triazolyl and the like.

The invention moreover relates to tautomers of compounds I as depicted. For instance, amide/imidic acid tautomerism in the depicted C(O)—NH group may be present. Analogously, tautomerism may be present if in ring A a NH ring member is adjacent to C═O or inversely ring A contains a moiety —C(OH)═N—. Also if X¹ is N and X² is C—OH or X² is N and X¹ or X³ is C—OH or X³ is N and X² or X⁴ is C—OH or X⁴ is N and X³ is C—OH, tautomerism may be present. Further, keto/enol tautomerism may be present if A contains a moiety —C(═O)—CH₂— or —C(═O)—CHR⁹— or —C(═O)—CHR¹⁰—or —C(OH)═CH— or —C(OH)═CR⁹— or —C(OH)═CR¹⁰—.

In addition to salt forms, the N-oxides, the salts of the N-oxides and the tautomers, the present invention provides compounds which are in a prodrug form. Prodrugs of the compounds described herein are those compounds that readily undergo chemical changes under physiological conditions to provide a compound of general formula (I). A prodrug is a pharmacologically active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters, see Svensson and Tunek, Drug Metabolism Reviews 16.5 (1988), and Bundgaard, Design of Prodrugs, Elsevier (1985). Examples of a masked acidic anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)). Hydroxy groups have been masked as esters and ethers. EP 0 039 051 (Sloan and Little, Apr. 11, 1981) discloses Mannich-base hydroxamic acid prodrugs, their preparation and use.

Certain compounds of the present invention can exist in unsolvated forms as well as in solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The compounds of the present invention may also contain unnatural proportions of atomic isotopes at one or more of the atoms that constitute such compounds. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulfur, fluorine and chlorine such as ²H, ³H, ¹³C, ¹⁴C, ¹⁵N, ¹⁷O, ¹⁸O ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents. All isotopic variations of the compounds and compositions of the present invention, whether radioactive or not, are intended to be encompassed within the scope of the present invention.

If L² is C₁-C₆-alkylene-O, C₁-C₆-alkylene-S, C₁-C₆-alkylene-NR¹⁵, C₃-C₈-cycloalkylene-O, C₃-C₈-cycloalkylene-S or C₃-C₈-cycloalkylene-NR¹⁵, O, S and NR¹⁵ are bound to the ring A.

The organic moieties mentioned in the above definitions of the variables are—like the term halogen—collective terms for individual listings of the individual group members. The prefix Cₙ-Cₘ indicates in each case the possible number of carbon atoms in the group. If two or more radicals can be selected independently from each other, then the term “independently” means that the radicals may be the same or may be different.

The term “halogen” denotes in each case fluorine, bromine, chlorine or iodine, in particular fluorine, chlorine or bromine. Halogen as a substituent on an aromatic or heteroaromatic group is preferably F or Cl, and on an aliphatic (e.g. on an alkyl, alkenyl, alkynyl, alkylene (derived) group) or cycloaliphatic (e.g. on a cycloalkyl group) group or on a saturated or partially unsaturated heterocyclic ring is F.

The term “alkyl” as used herein and in the alkyl moieties of alkoxy and the like refers to saturated straight-chain or branched hydrocarbon radicals having 1 to 2 (“C₁-C₂-alkyl”), 1 to 3 (“C₁-C₃-alkyl”), 1 to 4 (“C₁-C₄-alkyl”) or 1 to 6 (“C₁-C₆-alkyl”). C₁-C₂-Alkyl is methyl or ethyl. C₁-C₃-Alkyl is additionally propyl and isopropyl. C₁-C₄-Alkyl is additionally butyl, 1-methylpropyl (sec-butyl), 2-methylpropyl (isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl.

The term “haloalkyl” as used herein, which may also be expressed as “alkyl which is partially or fully halogenated”, refers to straight-chain or branched alkyl groups having 1 to 2 (“C₁-C₂-haloalkyl”), 1 to 3 (“C₁-C₃-haloalkyl”), 1 to 4 (“C₁-C₄-haloalkyl”) or 1 to 6 (“C₁-C₂-haloalkyl”) carbon atoms (as mentioned above), where some or all of the hydrogen atoms in these groups are replaced by fluorine atoms. Examples for C₁-C₂-haloalkyl (indeed for fluorinated C₁-C₂-alkyl) are fluoromethyl, difluoromethyl, trifluoromethyl, 1-fluoroethyl, 2-fluoroethyl, 2,2-difluoroethyl, 2,2,2-trifluoroethyl, or pentafluoroethyl. Examples for C₁-C₃-haloalkyl (indeed for fluorinated C₁-C₃-alkyl) are, in addition to those mentioned for C₁-C₂-haloalkyl, 1-fluoropropyl, 2-fluoropropyl, (R)-2-fluoropropyl, (S)-2-fluoropropyl, 3-fluoropropyl, 1,1-difluoropropyl, 2,2-difluoropropyl, 1,2-difluoropropyl, 2,3-difluoropropyl, 3,3-difluoropropyl, 2,2,3-trifluoropropyl, 3,3,3-trifluoropropyl, 2,2,3,3-tetrafluoropropyl, 2,2,3,3,3-pentafluoropropyl, heptafluoropropyl, 1,1,1-trifluoroprop-2-yl, 2-fluoro-1-methylethyl, (R)-2-fluoro-1-methylethyl, (S)-2-fluoro-1-methylethyl, 2,2-difluoro-1-methylethyl, (R)-2,2-difluoro-1-methylethyl, (S)-2,2-difluoro-1-methylethyl, 2,2,2-trifluoro-1-methylethyl, (R)-2,2,2-trifluoro-1-methylethyl, (S)-2,2,2-trifluoro-1-methylethyl, 2-fluoro-1-(fluoromethyl)ethyl, 1-(difluoromethyl)-2,2-difluoroethyl, 1-(trifluoromethyl)-2,2,2-trifluoroethyl, 1-(trifluoromethyl)-1,2,2,2-tetrafluoroethyl and the like. Examples for C₁-C₄-haloalkyl are, in addition to those mentioned for C₁-C₃-haloalkyl, 2-fluorobutyl, (R)-2-fluorobutyl, (S)-2-fluorobutyl, 3-fluorobutyl, (R)-3-fluorobutyl, (S)-3-fluorobutyl, 4-fluorobutyl, 2,2-difluorobutyl, 3,3-difluorobutyl, 4,4-difluorobutyl, 4,4,4-trifluorobutyl, 3,3,4,4-tetrafluorobutyl, 3,4,4,4-tetrafluorobutyl, 2,2,4,4,4-pentafluorobutyl, 3,3,4,4,4-pentafluorobutyl, 2,2,3,4,4,4-hexafluorobutyl, 1-methyl-2,2-3,3-tetrafluoropropyl and the like.

The term “alkenyl” as used herein refers to monounsaturated straight-chain or branched hydrocarbon radicals having 3 or 4 (“C₃-C₄-alkenyl”), 2 to 4 (“C₂-C₄-alkenyl”) or 2 to 6 (“C₂-C₆-alkenyl”) carbon atoms and a double bond in any position. Examples for C₃-C₄-alkenyl are 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl. Examples for C₂-C₄-alkenyl are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl. Examples for C₂-C₆-alkenyl are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl or 1-ethyl-2-methyl-2-propenyl.

The term “haloalkenyl” as used herein, which may also be expressed as “alkenyl which is partially or fully halogenated”, refers to unsaturated straight-chain or branched hydrocarbon radicals having 3 or 4 (“C₃-C₄-haloalkenyl”), 2 to 4 (“C₂-C₄-haloalkenyl”) or 2 to 6 (“C₂-C₆-haloalkenyl”) carbon atoms and a double bond in any position (as mentioned above), where some or all of the hydrogen atoms in these groups are replaced by fluorine atoms, for example fluorovinyl, fluoroallyl and the like.

The term “alkynyl” as used herein refers to straight-chain or branched hydrocarbon groups having 2 or 3 (“C₂-C₃-alkynyl”), 2 to 4 (“C₂-C₄-alkynyl”) or 2 to 6 (“C₂-C₆-alkynyl”) carbon atoms and one triple bond in any position. Examples for C₂-C₃-alkynyl are ethynyl, 1-propynyl or 2-propynyl. Examples for C₂-C₄-alkynyl are ethynyl, 1-propynyl, 2-propynyl, 1-butynyl, 2-butynyl, 3-butynyl or 1-methyl-2-propynyl. Examples for C₂-C₆-alkynyl are ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl or 1-ethyl-1-methyl-2-propynyl.

The term “haloalkynyl” as used herein, which can also be expressed as “alkynyl which is partially or fully halogenated”, refers to unsaturated straight-chain or branched hydrocarbon radicals having 2 or (“C₂-C₃-haloalkynyl”), 2 to 4 (“C₃-C₄-haloalkynyl”) or 2 to 6 (“C₂-C₆-haloalkynyl”) carbon atoms and one triple bond in any position (as mentioned above), where some or all of the hydrogen atoms in these groups are replaced by fluorine atoms.

The term “cycloalkyl” as used herein refers to mono- or bi- or polycyclic saturated hydrocarbon radicals having 3 to 8 (“C₃-C₈-cycloalkyl”), in particular 3 to 6 carbon atoms (“C₃-C₆-cycloalkyl”) or 5 or 6 carbon atoms (“C₅-C₆-cycloalkyl”). Examples of monocyclic radicals having 5 or 6 carbon atoms are cyclopentyl and cyclohexyl. Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl. Preferably, the term cycloalkyl denotes a monocyclic saturated hydrocarbon radical.

The term “halocycloalkyl” as used herein, which can also be expressed as “cycloalkyl which is partially or fully halogenated”, refers to mono- or bi- or polycyclic saturated hydrocarbon groups having 3 to 8 (“C₃-C₈-halocycloalkyl”) or preferably 3 to 6 (“C₃-C₆-halocycloalkyl”) or 5 or 6 (“C₅-C₆-halocycloalkyl”) carbon ring members (as mentioned above) in which some or all of the hydrogen atoms are replaced by fluorine atoms.

The term “cycloalkyl-C₁-C₄-alkyl” refers to a C₃-C₈-cycloalkyl group (“C₃-C₈-cycloalkyl-C₁-C₄-alkyl”), preferably a C₃-C₆-cycloalkyl group (“C₃-C₆-cycloalkyl-C₁-C₄-alkyl”), more preferably a C₃-C₄-cycloalkyl group (“C₃-C₄-cycloalkyl-C₁-C₄-alkyl”) as defined above (preferably a monocyclic cycloalkyl group) which is bound to the remainder of the molecule via a C₁-C₄-alkyl group, as defined above. Examples for C₃-C₄-cycloalkyl-C₁-C₄-alkyl are cyclopropylmethyl, cyclopropylethyl, cyclopropylpropyl, cyclobutylmethyl, cyclobutylethyl and cyclobutylpropyl, Examples for C₃-C₆-cycloalkyl-C₁-C₄-alkyl are, in addition to those mentioned for C₃-C₄-cycloalkyl-C₁-C₄-alkyl, cyclopentylmethyl, cyclopentylethyl, cyclopentylpropyl, cyclohexylmethyl, cyclohexylethyl and cyclohexylpropyl. Examples for C₃-C₈-cycloalkyl-C₁-C₄-alkyl are, in addition to those mentioned for C₃-C₆-cycloalkyl-C₁-C₄-alkyl, cycloheptylmethyl, cycloheptylethyl, cyclooctylmethyl and the like.

The term “C₃-C₈-halocycloalkyl-C₁-C₄-alkyl” refers to a C₃-C₈-halocycloalkyl group as defined above, i.e. to fluorinated C₃-C₈-cycloalkyl, which is bound to the remainder of the molecule via a C₁-C₄-alkyl group, as defined above.

The term “C₁-C₂-alkoxy” denotes a C₁-C₂-alkyl group, as defined above, attached via an oxygen atom to the remainder of the molecule. The term “C₁-C₃-alkoxy” denotes a C₁-C₃-alkyl group, as defined above, attached via an oxygen atom. The term “C₁-C₄-alkoxy” denotes a C₁-C₄-alkyl group, as defined above, attached via an oxygen atom. The term “C₁-C₆-alkoxy” denotes a C₁-C₆-alkyl group, as defined above, attached via an oxygen atom. C₁-C₂-Alkoxy is methoxy or ethoxy. C₁-C₃-Alkoxy is additionally, for example, n-propoxy or 1-methylethoxy (isopropoxy). C₁-C₄-Alkoxy is additionally, for example, butoxy, 1-methylpropoxy (sec-butoxy), 2-methylpropoxy (isobutoxy) or 1,1-dimethylethoxy (tert-butoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy.

The term “C₁-C₂-haloalkoxy” denotes a C₁-C₂-haloalkyl group, as defined above, attached via an oxygen atom to the remainder of the molecule. The term “C₁-C₃-haloalkoxy” denotes a C₁-C₃-haloalkyl group, as defined above, attached via an oxygen atom. The term “C₁-C₄-haloalkoxy” denotes a C₁-C₄-haloalkyl group, as defined above, attached via an oxygen atom. The term “C₁-C₆-haloalkoxy” denotes a C₁-C₆-haloalkyl group, as defined above, attached via an oxygen atom. C₁-C₂-Haloalkoxy (indeed fluorinated C₁-C₂-alkoxy) is, for example, OCH₂F, OCHF₂, OCF₃, 2-fluoroethoxy, 2-2,2-difluoroethoxy, 2,2,2-trifluoroethoxy or OC₂F₅. C₁-C₃-Haloalkoxy (indeed fluorinated C₁-C₃-alkoxy) is additionally, for example, 2-fluoropropoxy, 3-fluoropropoxy, 2,2-difluoropropoxy, 2,3-difluoropropoxy, 3,3,3-trifluoropropoxy, OCH₂—C₂F₅, OCF₂—C₂F₅ or 1-(CH₂F)-2-fluoroethoxy. C₁-C₄-Haloalkoxy (indeed fluorinated C₁-C₄-alkoxy) is additionally, for example, 4-fluorobutoxy or nonafluorobutoxy. C₁-C₆-Haloalkoxy (indeed fluorinated C₁-C₆-alkoxy) is additionally, for example, 5-fluoropentoxy, undecafluoropentoxy, 6-fluorohexoxy or dodecafluorohexoxy.

The term “C₁-C₄-alkoxy-C₁-C₄-alkyl” as used herein, refers to a straight-chain or branched alkyl group having 1 to 4 carbon atoms, as defined above, where one hydrogen atom is replaced by a C₁-C₄-alkoxy group, as defined above. The term “C₁-C₆-alkoxy-C₁-C₆-alkyl” as used herein, refers to a straight-chain or branched alkyl group having 1 to 6 carbon atoms, as defined above, where one hydrogen atom is replaced by a C₁-C₆-alkoxy group, as defined above. Examples are methoxymethyl, ethoxymethyl, propoxymethyl, isopropoxymethyl, n-butoxymethyl, sec-butoxymethyl, isobutoxymethyl, tert-butoxymethyl, 1-methoxyethyl, 1-ethoxyethyl, 1-propoxyethyl, 1-isopropoxyethyl, 1-n-butoxyethyl, 1-sec-butoxyethyl, 1-isobutoxyethyl, 1-tert-butoxyethyl, 2-methoxyethyl, 2-ethoxyethyl, 2-propoxyethyl, 2-isopropoxyethyl, 2-n-butoxyethyl, 2-sec-butoxyethyl, 2-isobutoxyethyl, 2-tert-butoxyethyl, 1-methoxypropyl, 1-ethoxypropyl, 1-propoxypropyl, 1-isopropoxypropyl, 1-n-butoxypropyl, 1-sec-butoxypropyl, 1-isobutoxypropyl, 1-tert-butoxypropyl, 2-methoxypropyl, 2-ethoxypropyl, 2-propoxypropyl, 2-isopropoxypropyl, 2-n-butoxypropyl, 2-sec-butoxypropyl, 2-isobutoxypropyl, 2-tert-butoxypropyl, 3-methoxypropyl, 3-ethoxypropyl, 3-propoxypropyl, 3-isopropoxypropyl, 3-n-butoxypropyl, 3-sec-butoxypropyl, 3-isobutoxypropyl, 3-tert-butoxypropyl and the like.

C₁-C₆-Haloalkoxy-C₁-C₆-alkyl is a straight-chain or branched alkyl group having from 1 to 6, especially 1 to 4 carbon atoms (═C₁-C₆-haloalkoxy-C₁-C₄-alkyl), wherein one of the hydrogen atoms is replaced by a C₁-C₆-alkoxy group and wherein at least one, e.g. 1, 2, 3, 4 or all of the remaining hydrogen atoms (either in the alkoxy moiety or in the alkyl moiety or in both) are replaced by fluorine atoms. C₁-C₄-Haloalkoxy-C₁-C₄-alkyl (indeed fluorinated C₁-C₄-alkoxy-C₁-C₄-alkyl) is a straight-chain or branched alkyl group having from 1 to 4 carbon atoms, wherein one of the hydrogen atoms is replaced by a C₁-C₄-alkoxy group and wherein at least one, e.g. 1, 2, 3, 4 or all of the remaining hydrogen atoms (either in the alkoxy moiety or in the alkyl moiety or in both) are replaced by fluorine atoms. Examples are difluoromethoxymethyl (CHF₂OCH₂), trifluoromethoxymethyl, 1-difluoromethoxyethyl, 1-trifluoromethoxyethyl, 2-difluoromethoxyethyl, 2-trifluoromethoxyethyl, difluoro-methoxy-methyl (CH₃OCF₂), 1,1-difluoro-2-methoxyethyl, 2,2-difluoro-2-methoxyethyl and the like.

The term “C₁-C₂-alkylthio” denotes a C₁-C₂-alkyl group, as defined above, attached via a sulfur atom to the remainder of the molecule. The term “C₁-C₃-alkylthio” denotes a C₁-C₃-alkyl group, as defined above, attached via a sulfur atom. The term “C₁-C₄-alkylthio” denotes a C₁-C₄-alkyl group, as defined above, attached via a sulfur atom. The term “C₁-C₆-alkylthio” denotes a C₁-C₆-alkyl group, as defined above, attached via a sulfur atom. C₁-C₂-Alkylthio is methylthio or ethylthio. C₁-C₃-Alkylthio is additionally, for example, n-propylthio or 1-methylethylthio (isopropylthio). C₁-C₄-Alkylthio is additionally, for example, butylthio, 1-methylpropylthio (sec-butylthio), 2-methylpropylthio (isobutylthio) or 1,1-dimethylethylthio (tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio.

The term “C₁-C₂-haloalkylthio” denotes a C₁-C₂-haloalkyl group, as defined above, attached via a sulfur atom to the remainder of the molecule. The term “C₁-C₃-haloalkylthio” denotes a C₁-C₃-haloalkyl group, as defined above, attached via a sulfur atom. The term “C₁-C₄-haloalkylthio” denotes a C₁-C₄-haloalkyl group, as defined above, attached via a sulfur atom. The term “C₁-C₆-haloalkylthio” denotes a C₁-C₆-haloalkyl group, as defined above, attached via a sulfur atom. C₁-C₂-Haloalkylthio (indeed fluorinated C₁-C₂-alkylthio) is, for example, SCH₂F, SCHF₂, SCF₃, 2-fluoroethylthio, 2,2-difluoroethylthio, or SCZFs. C₁-C₃-Haloalkylthio (indeed fluorinated C₁-C₃-alkylthio) is additionally, for example, 2-fluoropropylthio, 3-fluoropropylthio, 2,2-difluoropropylthio, 2,3-difluoropropylthio, 3,3,3-trifluoropropylthio, SCH₂—C₂F₅, SCF₂—C₂F₅ or 1-(CH₂F)-2-fluoroethylthio, C₁-C₄-Haloalkylthio (indeed fluorinated C₁-C₄-alkylthio) is additionally, for example, 4-fluorobutylthio or nonafluorobutylthio. C₁-C₆-Haloalkylthio (indeed fluorinated C₁-C₆-alkylthio) is additionally, for example, 5-fluoropentylthio, undecafluoropentylthio, 6-fluorohexylthio or dodecafluorohexylthio.

The term “C₁-C₂-alkylsulfonyl” denotes a C₁-C₂-alkyl group, as defined above, attached via a sulfonyl [S(O)₂] group to the remainder of the molecule. The term “C₁-C₃-alkylsulfonyl” denotes a C₁-C₃-alkyl group, as defined above, attached via a sulfonyl [S(O)₂] group. The term “C₁-C₄-alkylsulfonyl” denotes a C₁-C₄-alkyl group, as defined above, attached via a sulfonyl [S(O)₂] group. The term “C₁-C₆-alkylsulfonyl” denotes a C₁-C₆-alkyl group, as defined above, attached via a sulfonyl [S(O)₂] group. C₁-C₂-Alkylsulfonyl is methylsulfonyl or ethylsulfonyl. C₁-C₃-Alkylsulfonyl is additionally, for example, n-propylsulfonyl or 1-methylethylsulfonyl (isopropylsulfonyl). C₁-C₄-Alkylsulfonyl is additionally, for example, butylsulfonyl, 1-methylpropylsulfonyl (sec-butylsulfonyl), 2-methylpropylsulfonyl (isobutylsulfonyl) or 1,1-dimethylethylsulfonyl (tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” denotes a C₁-C₂-haloalkyl group, as defined above, attached via a sulfonyl [S(O)₂] group to the remainder of the molecule. The term “C₁-C₃-haloalkylsulfonyl” denotes a C₁-C₃-haloalkyl group, as defined above, attached via a sulfonyl [S(O)₂] group. The term “C₁-C₄-haloalkylsulfonyl” denotes a C₁-C₄-haloalkyl group, as defined above, attached via a sulfonyl [S(O)₂] group. The term “C₁-C₆-haloalkylsulfonyl” denotes a C₁-C₆-haloalkyl group, as defined above, attached via a sulfonyl [S(O)₂] group. C₁-C₂-Haloalkylsulfonyl (indeed fluorinated C₁-C₂-alkylsulfonyl) is, for example, S(O)₂CH₂F, S(O)₂CHF₂, S(O)₂CF₃, 2-fluoroethylsulfonyl, 2,2-difluoroethylsulfonyl, 2,2,2-trifluoroethylsulfonyl or S(O)₂C₂F₅. C₁-C₃-Haloalkylsulfonyl (indeed fluorinated C₁-C₃-alkylsulfonyl) is additionally, for example, 2-fluoropropylsulfonyl, 3-fluoropropylsulfonyl, 2,2-difluoropropylsulfonyl, 2,3-difluoropropylsulfonyl, 3,3,3-trifluoropropylsulfonyl, S(O)₂CH₂—C₂F₅, S(O)₂CF₂—C₂F₅ or 1-(CH₂F)-2-fluoroethylsulfonyl. C₁-C₄-Haloalkylsulfonyl (indeed fluorinated C₁-C₄-alkylsulfonyl) is additionally, for example, 4-fluorobutylsulfonyl or nonafluorobutylsulfonyl. C₁-C₆-Haloalkylsulfonyl (indeed fluorinated C₁-C₆-alkylsulfonyl) is additionally, for example, 5-fluoropentylsulfonyl, undecafluoropentylsulfonyl, 6-fluorohexylsulfonyl or dodecafluorohexylsulfonyl.

The substituent “oxo” is ═O; i.e. it replaces a CH₂ group by a C(═O) group.

“Carboxyl” is —C(═O)OH group.

The term “alkylcarbonyl” denotes a C₁-C₆-alkyl (“C₁-C₆-alkylcarbonyl”), preferably a C₁-C₄-alkyl (“C₁-C₄-alkylcarbonyl”) group, as defined above, attached to the remainder of the molecule via a carbonyl [C(═O)] group. Examples are acetyl (methylcarbonyl), propionyl (ethylcarbonyl), propylcarbonyl, isopropylcarbonyl, n-butylcarbonyl and the like.

The term “haloalkylcarbonyl” denotes a C₁-C₆-haloalkyl (“C₁-C₆-haloalkylcarbonyl”; indeed fluorinated C₁-C₆-alkylcarbonyl), preferably a C₁-C₄-haloalkyl (“C₁-C₄-haloalkylcarbonyl”; indeed fluorinated C₁-C₄-alkylcarbonyl) group, as defined above, attached to the remainder of the molecule via a carbonyl [C(═O)] group. Examples are trifluoromethylcarbonyl, 2,2,2-trifluoroethylcarbonyl and the like.

The term “alkoxycarbonyl” denotes a C₁-C₆-alkoxy (“C₁-C₆-alkoxycarbonyl”), preferably a C₁-C₄-alkoxy (“C₁-C₄-alkoxycarbonyl”) group, as defined above, attached to the remainder of the molecule via a carbonyl [C(═O)] group. Examples are methoxycarbonyl), ethoxycarbonyl, propoxycarbonyl, isopropoxycarbonyl, n-butoxycarbonyl and the like.

The term “haloalkoxycarbonyl” denotes a C₁-C₆-haloalkoxy (“C₁-C₆-haloalkoxycarbonyl”; indeed fluorinated C₁-C₆-alkoxycarbonyl), preferably a C₁-C₄-haloalkoxy (“C₁-C₄-haloalkoxycarbonyl”; indeed fluorinated C₁-C₄-alkoxycarbonyl) group, as defined above, attached to the remainder of the molecule via a carbonyl [C(═O)] group. Examples are trifluoromethoxycarbonyl, 2,2,2-trifluoroethoxycarbonyl and the like.

The term “3-, 4-, 5-, 6-, 7- or 8-membered saturated, partially unsaturated or maximally unsaturated carbocyclic ring” as used herein denotes monocyclic radicals containing only C atoms as ring members, the monocyclic radicals being saturated, partially unsaturated or maximum unsaturated (including aromatic).

Unsaturated carbocyclic rings contain at least one C—C double bond. Maximally unsaturated rings contain as many conjugated C—C double bonds as allowed by the ring size. Partially unsaturated rings contain less than the maximum number of C—C double bond(s) allowed by the ring size.

A 3-, 4-, 5-, 6-, 7- or 8-membered saturated unsaturated carbocyclic ring is C₃-C₈-cycloalkyl, as defined above.

Examples for 3-, 4-, 5-, 6-, 7- or 8-membered partially unsaturated carbocyclic rings are cyclobut-1-en-1-yl, cyclobut-1-en-3-yl, cyclopent-1-en-1-yl, cyclopent-1-en-3-yl, cyclopent-1-en-4-yl, cyclopenta-1,3-dien-1-yl, cyclopenta-1,3-dien-2-yl, cyclopenta-1,3-dien-5-yl, cyclohex-1-en-1-yl, cyclohex-1-en-3-yl, cyclohex-1-en-4-yl, cyclohexa-1,3-dien-1-yl, cyclohexa-1,3-dien-2-yl, cyclohexa-1,3-dien-5-yl, cyclohexa-1,4-dien-1-yl, cyclohexa-1,4-dien-3-yl, cyclohept-1-en-1-yl, cyclohept-1-en-3-yl, cyclohept-1-en-4-yl, cyclohept-1-en-5-yl, cyclohepta-1,3-dien-1-yl, cyclohepta-1,3-dien-2-yl, cyclohepta-1,3-dien-5-yl, cyclohepta-1,3-dien-6-yl, cyclohepta-1,4-dien-1-yl, cyclohepta-1,4-dien-2-yl, cyclohepta-1,4-dien-3-yl, cyclohepta-1,4-dien-6-yl, cyclooct-1-en-1-yl, cyclooct-1-en-3-yl, cyclooct-1-en-4-yl, cyclooct-1-en-5-yl, cycloocta-1,3-dien-1-yl, cycloocta-1,3-dien-2-yl, cycloocta-1,3-dien-5-yl, cycloocta-1,3-dien-6-yl, cycloocta-1,4-dien-1-yl, cycloocta-1,4-dien-2-yl, cycloocta-1,4-dien-3-yl, cycloocta-1,4-dien-6-yl, cycloocta-1,4-dien-7-yl, cycloocta-1,5-dien-1-yl, and cycloocta-1,5-dien-3-yl.

Examples for 3-, 4-, 5-, 6-, 7- or 8-membered maximally unsaturated carbocyclic rings are cycloprop-1-en-1-yl, cycloprop-1-en-3-yl, cyclobutadienyl, cyclopenta-1,3-dien-1-yl, cyclopenta-1,3-dien-2-yl, cyclopenta-1,3-dien-5-yl, phenyl, cyclohepta-1,3,5-trien-1-yl, cyclohepta-1,3,5-trien-2-yl, cyclohepta-1,3,5-trien-3-yl, cyclohepta-1,3,5-trien-7-yl and cyclooctatetraenyl.

Aryl is an aromatic carbocyclic ring containing 6 to 14 carbon atoms. Examples are phenyl, naphthyl, phenanthrenyl and anthracenyl.

The term “aryl-C₁-C₃-alkyl” refers to an aryl group, as defined above, bound to the remainder of the molecule via a C₁-C₃-alkyl group. Examples are benzyl, 1-phenylethyl, 2-phenylethyl (phenethyl), 1-phenylpropyl, 2-phenylpropyl, 3-phenylpropyl, naphth-1-yl-methyl or naphth-2-yl-methyl.

The term “3-, 4-, 5-, 6-, 7- or 8-membered saturated, partially unsaturated or maximally unsaturated heterocyclic ring containing 1, 2, 3 or 4 heteroatoms or heteroatom groups selected from the group consisting of O, N, S, NO, SO and SO₂, as ring members” [wherein “maximum unsaturated” includes also “aromatic” ] as used herein denotes monocyclic radicals, the monocyclic radicals being saturated, partially unsaturated or maximum unsaturated (including aromatic).

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heteromonocyclic rings are generally aromatic. Exceptions are maximally unsaturated 6-membered rings containing O, S, SO and/or SO₂ as ring members, such as pyran and thiopyran, which are not aromatic. Partially unsaturated rings contain less than the maximum number of C—C and/or C—N and/or N—N double bond(s) allowed by the ring size. The heterocyclic ring may be attached to the remainder of the molecule via a carbon ring member or via a nitrogen ring member. As a matter of course, the heterocyclic ring contains at least one carbon ring atom. If the ring contains more than one 0 ring atom, these are not adjacent.

Examples of a 3-, 4-, 5-, 6-, 7- or 8-membered saturated heteromonocyclic ring containing 1, 2, 3 or 4 heteroatoms or heteroatom groups selected from the group consisting of O, N, S, NO, SO and SO₂, as ring members include: Oxiran-2-yl, thiiran-2-yl, aziridin-1-yl, aziridin-2-yl, oxetan-2-yl, oxetan-3-yl, thietan-2-yl, thietan-3-yl, 1-oxothietan-2-yl, 1-oxothietan-3-yl, 1,1-dioxothietan-2-yl, 1,1-dioxothietan-3-yl, azetidin-1-yl, azetidin-2-yl, azetidin-3-yl, tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-2-yl, 1,2,4-oxadiazolidin-3-yl, 1,2,4-oxadiazolidin-4-yl, 1,2,4-oxadiazolidin-5-yl, 1,2,4-thiadiazolidin-2-yl, 1,2,4-thiadiazolidin-3-yl, 1,2,4-thiadiazolidin-4-yl, 1,2,4-thiadiazolidin-5-yl, 1,2,4-triazolidin-1-yl, 1,2,4-triazolidin-3-yl, 1,2,4-triazolidin-4-yl, 1,3,4-oxadiazolidin-2-yl, 1,3,4-oxadiazolidin-3-yl, 1,3,4-thiadiazolidin-2-yl, 1,3,4-thiadiazolidin-3-yl, 1,3,4-triazolidin-1-yl, 1,3,4-triazolidin-2-yl, 1,3,4-triazolidin-3-yl, 1,2,3,4-tetrazolidin-1-yl, 1,2,3,4-tetrazolidin-2-yl, 1,2,3,4-tetrazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl, 1,2,4-hexahydrotriazin-1-yl, 1,2,4-hexahydrotriazin-2-yl, 1,2,4-hexahydrotriazin-3-yl, 1,2,4-hexahydrotriazin-4-yl, 1,2,4-hexahydrotriazin-5-yl, 1,2,4-hexahydrotriazin-6-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, oxocane, thiocane, azocanyl, [1,3]diazocanyl, [1,4]diazocanyl, [1,5]diazocanyl, [1,5]oxazocanyl and the like.

Examples of a 3-, 4-, 5-, 6-, 7- or 8-membered partially unsaturated heteromonocyclic ring containing 1, 2, 3 or 4 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl, tetrahydro-1,4-dioxepinyl, 1,2,3,4,5,6-hexahydroazocine, 2,3,4,5,6,7-hexahydroazocine, 1,2,3,4,5,8-hexahydroazocine, 1,2,3,4,7,8-hexahydroazocine, 1,2,3,4,5,6-hexahydro-[1,5]diazocine,1,2,3,4,7,8-hexahydro-[1,5]diazocine and the like.

Examples of a 3-, 4-, 5-, 6-, 7- or 8-membered maximally unsaturated (including aromatic) heteromonocyclic ring containing 1, 2, 3 or 4 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, 2H-oxazin-2-yl, 2H-oxazin-3-yl, 2H-oxazin-4-yl, 2H-oxazin-5-yl, 2H-oxazin-6-yl, 4H-oxazin-3-yl, 4H-oxazin-4-yl, 4H-oxazin-5-yl, 4H-oxazin-6-yl, 6H-oxazin-3-yl, 6H-oxazin-4-yl, 7H-oxazin-5-yl, 8H-oxazin-6-yl, 2H-1,3-oxazin-2-yl, 2H-1,3-oxazin-4-yl, 2H-1,3-oxazin-5-yl, 2H-1,3-oxazin-6-yl, 4H-1,3-oxazin-2-yl, 4H-1,3-oxazin-4-yl, 4H-1,3-oxazin-5-yl, 4H-1,3-oxazin-6-yl, 6H-1,3-oxazin-2-yl, 6H-1,3-oxazin-4-yl, 6H-1,3-oxazin-5-yl, 6H-1,3-oxazin-6-yl, 2H-1,4-oxazin-2-yl, 2H-1,4-oxazin-3-yl, 2H-1,4-oxazin-5-yl, 2H-1,4-oxazin-6-yl, 4H-1,4-oxazin-2-yl, 4H-1,4-oxazin-3-yl, 4H-1,4-oxazin-4-yl, 4H-1,4-oxazin-5-yl, 4H-1,4-oxazin-6-yl, 6H-1,4-oxazin-2-yl, 6H-1,4-oxazin-3-yl, 6H-1,4-oxazin-5-yl, 6H-1,4-oxazin-6-yl, 1,4-dioxine-2-yl, 1,4-oxathiin-2-yl, 1H-azepine, 1H-[1,3]-diazepine, 1H-[1,4]-diazepine, [1,3]diazocine, [1,5]diazocine, [1,5]diazocine and the like.

Examples of a 3-, 4-, 5-, 6-, 7- or 8-membered saturated heteromonocyclic ring containing 1 or 2 heteroatoms or heteroatom groups selected from the group consisting of O, N, S, NO, SO and SO₂, as ring members include: Oxiran-2-yl, thiiran-2-yl, aziridin-1-yl, aziridin-2-yl, oxetan-2-yl, oxetan-3-yl, thietan-2-yl, thietan-3-yl, 1-oxothietan-2-yl, 1-oxothietan-3-yl, 1,1-dioxothietan-2-yl, 1,1-dioxothietan-3-yl, azetidin-1-yl, azetidin-2-yl, azetidin-3-yl, tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, oxocane, thiocane, azocanyl, [1,3]diazocanyl, [1,4]diazocanyl, [1,5]diazocanyl, [1,5]oxazocanyl and the like.

Examples of a 3-, 4-, 5-, 6-, 7- or 8-membered partially unsaturated heteromonocyclic ring containing 1 or 2 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl, tetrahydro-1,4-dioxepinyl, 1,2,3,4,5,6-hexahydroazocine, 2,3,4,5,6,7-hexahydroazocine, 1,2,3,4,5,8-hexahydroazocine, 1,2,3,4,7,8-hexahydroazocine, 1,2,3,4,5,6-hexahydro-[1,5]diazocine,1,2,3,4,7,8-hexahydro-[1,5]diazocine and the like.

Examples of a 3-, 4-, 5-, 6-, 7- or 8-membered maximally unsaturated (including aromatic) heteromonocyclic ring containing 1 or 2 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, 2H-oxazin-2-yl, 2H-oxazin-3-yl, 2H-oxazin-4-yl, 2H-oxazin-5-yl, 2H-oxazin-6-yl, 4H-oxazin-3-yl, 4H-oxazin-4-yl, 4H-oxazin-5-yl, 4H-oxazin-6-yl, 6H-oxazin-3-yl, 6H-oxazin-4-yl, 7H-oxazin-5-yl, 8H-oxazin-6-yl, 2H-1,3-oxazin-2-yl, 2H-1,3-oxazin-4-yl, 2H-1,3-oxazin-5-yl, 2H-1,3-oxazin-6-yl, 4H-1,3-oxazin-2-yl, 4H-1,3-oxazin-4-yl, 4H-1,3-oxazin-5-yl, 4H-1,3-oxazin-6-yl, 6H-1,3-oxazin-2-yl, 6H-1,3-oxazin-4-yl, 6H-1,3-oxazin-5-yl, 6H-1,3-oxazin-6-yl, 2H-1,4-oxazin-2-yl, 2H-1,4-oxazin-3-yl, 2H-1,4-oxazin-5-yl, 2H-1,4-oxazin-6-yl, 4H-1,4-oxazin-2-yl, 4H-1,4-oxazin-3-yl, 4H-1,4-oxazin-4-yl, 4H-1,4-oxazin-5-yl, 4H-1,4-oxazin-6-yl, 6H-1,4-oxazin-2-yl, 6H-1,4-oxazin-3-yl, 6H-1,4-oxazin-5-yl, 6H-1,4-oxazin-6-yl, 1,4-dioxine-2-yl, 1,4-oxathiin-2-yl, 1H-azepine, 1H-[1,3]-diazepine, 1H-[1,4]-diazepine, [1,3]diazocine, [1,5]diazocine, [1,5]diazocine and the like.

Examples of a 5- or 6-membered saturated heteromonocyclic ring containing 1, 2, 3 or 4 heteroatoms or heteroatom groups selected from the group consisting of O, N, S, NO, SO and SO₂, as ring members include: tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-2-yl, 1,2,4-oxadiazolidin-3-yl, 1,2,4-oxadiazolidin-4-yl, 1,2,4-oxadiazolidin-5-yl, 1,2,4-thiadiazolidin-2-yl, 1,2,4-thiadiazolidin-3-yl, 1,2,4-thiadiazolidin-4-yl, 1,2,4-thiadiazolidin-5-yl, 1,2,4-triazolidin-1-yl, 1,2,4-triazolidin-3-yl, 1,2,4-triazolidin-4-yl, 1,3,4-oxadiazolidin-2-yl, 1,3,4-oxadiazolidin-3-yl, 1,3,4-thiadiazolidin-2-yl, 1,3,4-thiadiazolidin-3-yl, 1,3,4-triazolidin-1-yl, 1,3,4-triazolidin-2-yl, 1,3,4-triazolidin-3-yl, 1,2,3,4-tetrazolidin-1-yl, 1,2,3,4-tetrazolidin-2-yl, 1,2,3,4-tetrazolidin-5-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl, 1,2,4-hexahydrotriazin-1-yl, 1,2,4-hexahydrotriazin-2-yl, 1,2,4-hexahydrotriazin-3-yl, 1,2,4-hexahydrotriazin-4-yl, 1,2,4-hexahydrotriazin-5-yl, 1,2,4-hexahydrotriazin-6-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, and the like.

Examples of a 5- or 6-membered partially unsaturated heteromonocyclic ring containing 1, 2, 3 or 4 heteroatoms or heteroatom groups selected from the group consisting of O, N, S, NO, SO and SO₂, as ring members include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, and the like.

Examples of a 5- or 6-membered maximally unsaturated (including aromatic) heteromonocyclic ring containing 1, 2, 3 or 4 heteroatoms or heteroatom groups selected from the group consisting of O, N, S, NO, SO and SO₂, as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 1,2,3,4-tetrazol-1-yl, 1,2,3,4-tetrazol-2-yl, 1,2,3,4-tetrazol-5-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl, pyran-2-yl, pyran-3-yl, pyran-4-yl, thiopyran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, and the like.

Examples for 5- or 6-membered monocyclic heteroaromatic rings containing 1, 2, 3 or 4 heteroatoms selected from the group consisting of N, O and S as ring members are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl and the like.

Examples for 5- or 6-membered monocyclic heteroaromatic rings containing 1 heteroatom selected from the group consisting of N, O and S as ring member are 2-furyl, 3-furyl, 2-thienyl, 3-thienyl, 1-pyrrolyl, 2-pyrrolyl, 3-pyrrolyl, 2-pyridinyl, 3-pyridinyl and 4-pyridinyl.

Examples for a 5-membered monocyclic heteroaromatic ring containing 1 heteroatom selected from the group consisting of N, O and S as ring member are 2-furyl, 3-furyl, 2-thienyl, 3-thienyl, 1-pyrrolyl, 2-pyrrolyl and 3-pyrrolyl.

“Hetaryl-C₁-C₃-alkyl” refers to a 5- or 6-membered heteroaromatic ring containing 1, 2, 3, or 4 heteroatoms selected from the group consisting of O, S and N as ring members, as defined above, bound to the remainder of the molecule via a C₁-C₃-alkyl group. Examples are 2-furyl-methyl, 3-furyl-methyl, 2-thienyl-methyl, 3-thienyl-methyl, 1-pyrrolyl-methyl, 2-pyrrolyl-methyl, 3-pyrrolyl-methyl, 1-pyrazolyl-methyl, 3-pyrazolyl-methyl, 4-pyrazolyl-methyl, 5-pyrazolyl-methyl, 1-imidazolyl-methyl, 2-imidazolyl-methyl, 4-imidazolyl-methyl, 5-imidazolyl-methyl, 2-oxazolyl-methyl, 4-oxazolyl-methyl, 5-oxazolyl-methyl, 3-isoxazolyl-methyl, 4-isoxazolyl-methyl, 5-isoxazolyl-methyl, 2-thiazolyl-methyl, 4-thiazolyl-methyl, 5-thiazolyl-methyl, 3-isothiazolyl-methyl, 4-isothiazolyl-methyl, 5-isothiazolyl-methyl, 1,3,4-triazol-1-yl-methyl, 1,3,4-triazol-2-yl-methyl, 1,3,4-triazol-3-yl-methyl, 1,2,3-triazol-1-yl-methyl, 1,2,3-triazol-2-yl-methyl, 1,2,3-triazol-4-yl-methyl, 1,2,5-oxadiazol-3-yl-methyl, 1,2,3-oxadiazol-4-yl-methyl, 1,2,3-oxadiazol-5-yl-methyl, 1,3,4-oxadiazol-2-yl-methyl, 1,2,5-thiadiazol-3-yl-methyl, 1,2,3-thiadiazol-4-yl-methyl, 1,2,3-thiadiazol-5-yl-methyl, 1,3,4-thiadiazol-2-yl-methyl, 2-pyridinyl-methyl, 3-pyridinyl-methyl, 4-pyridinyl-methyl, 3-pyridazinyl-methyl, 4-pyridazinyl-methyl, 2-pyrimidinyl-methyl, 4-pyrimidinyl-methyl, 5-pyrimidinyl-methyl, 2-pyrazinyl-methyl, 1,3,5-triazin-2-yl-methyl, 1,2,4-triazin-3-yl-methyl, 1,2,4-triazin-5-yl-methyl, 1,2,3,4-tetrazin-1-yl-methyl, 1,2,3,4-tetrazin-2-yl-methyl, 1,2,3,4-tetrazin-5-yl-methyl and the like.

“Heterocyclyl-C₁-C₃-alkyl” is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, as defined above, bound to the remainder of the molecule via a C₁-C₃-alkyl group.

“Alkylene” is a linear or branched divalent alkanediyl radical. C₁-C₆-Alkylene is a linear or branched divalent alkyl radical having 1, 2, 3, 4, 5 or 6 carbon atoms. Examples are —CH₂—, —CH₂CH₂—, —CH(CH₃)—, —CH₂CH₂CH₂—, —CH(CH₃)CH₂—, —CH₂CH(CH₃)—, —C(CH₃)₂—, —CH₂CH₂CH₂CH₂—, —CH(CH₃)CH₂CH₂—, —CH₂CH₂CH(CH₃)—, —C(CH₃)₂CH₂—, —CH₂C(CH₃)₂—, —(CH₂)₅—, —(CH₂)₆—, —(CH₂)₇—, —(CH₂)₈—, —(CH₂)₉—, —(CH₂)₁₀— and positional isomers thereof.

“C₃-C₈-Cycloalkylene” stands for a divalent monocyclic, saturated hydrocarbon group having 3 to 8 carbon ring members. Examples are cyclopropane-1,1-diyl, cyclopropane-1,2-diyl, cyclobutane-1,1-diyl, cyclobutane-1,2-diyl, cyclobutane-1,3-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, cycloheptane-1,1-diyl, cycloheptane-1,2-diyl, cycloheptane-1,3-diyl, cycloheptane-1,4-diyl, cyclooctane-1,1-diyl, cyclooctane-1,2-diyl, cyclooctane-1,3-diyl, cyclooctane-1,4-diyl, and cyclooctane-1,5-diyl.

The remarks made above and in the following with respect to preferred aspects of the invention, e.g. to preferred meanings of the variables A, X¹, X², X³, X⁴, Y¹, Y², Z, L¹, L², R¹, R², R³, R⁴, R⁵, R^{5a}, R^{5b}, R^{5c}, R^{5d}, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴, m and n of compounds I, to preferred compounds I and to preferred embodiments of the methods or the use according to the invention, apply in each case on their own or in particular to combinations thereof.

In one embodiment, X¹ is CR¹, X² is CR², X³ is CR³ and X⁴ is CR⁴. In another embodiment, X¹ is N, X² is CR², X³ is CR³ and X⁴ is CR⁴. In yet another embodiment, X¹ is CR¹, X² is N, X³ is CR³ and X⁴ is CR⁴. In yet another embodiment, X¹ is CR¹, X² is CR², X³ is N and X⁴ is CR⁴. In yet another embodiment, X¹ is CR¹, X² is CR², X³ is CR³ and X⁴ is N. In yet another embodiment, X¹ is N, X² is CR², X³ is N and X⁴ is CR⁴. In yet another embodiment, X¹ is CR¹, X² is N, X³ is CR³ and X⁴ is N.

Preferably,

X¹ is CR¹, X² is CR², X³ is CR³ and X⁴ is CR⁴; or

X¹ is N, X² is CR², X³ is CR³ and X⁴ is CR⁴; or

X¹ is CR¹, X² is N, X³ is CR³ and X⁴ is CR⁴; or

X¹ is CR¹, X² is CR², X³ is N and X⁴ is CR⁴; or

X¹ is CR¹, X² is CR², X³ is CR³ and X⁴ is N.

In particular, X¹ is CR¹, X² is CR², X³ is CR³ and X⁴ is CR⁴.

Preferably,
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸, and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO, as ring members, where the heterocyclic ring may carry one or more substituents R¹⁸; and
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy;
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO, as ring members.

More preferably,
R¹ and R², independently of each other, are selected from the group consisting of hydrogen, halogen, CN, C₁-C₄-alkyl and C₁-C₄-alkoxy; and
R³ and R⁴, independently of each other, are selected from the group consisting of hydrogen, F, C₁-C₄-alkyl and C₁-C₄-alkoxy;
or R¹ and R², or R² and R³ form together a bridging group —CH₂CH₂CH₂—, —CH₂CH₂CH₂CH₂—, or —O—CH₂—O—.

Even more preferably,
R¹ and R², independently of each other, are selected from the group consisting of hydrogen, F, Cl, CN and C₁-C₄-alkyl; and
R³ and R⁴ are hydrogen;
or R¹ and R², or R² and R³ form together a bridging group —CH₂CH₂CH₂—, —CH₂CH₂CH₂CH₂—, or —O—CH₂—O—.

In particular,
R¹ and R², independently of each other, are selected from the group consisting of hydrogen, F, Cl, CN and C₁-C₄-alkyl;
R³ and R⁴ are hydrogen;
or R¹ and R², or R² and R³ form together a bridging group —CH₂CH₂CH₂—.

Specifically,
R¹ and R², independently of each other, are selected from the group consisting of hydrogen, F, Cl and C₁-C₄-alkyl; and
R³ and R⁴ are hydrogen.

More specifically,
R¹ and R², independently of each other, are selected from the group consisting of hydrogen, Cl and C₁-C₄-alkyl; in particular hydrogen, Cl and methyl; and
R³ and R⁴ are hydrogen.

Very specifically,
R¹ and R², independently of each other, are selected from the group consisting of hydrogen and C₁-C₄-alkyl; in particular hydrogen and methyl; and
R³ and R⁴ are hydrogen.

Even more specifically,

R² is selected from the group consisting of hydrogen, Cl and C₁-C₄-alkyl; and

R¹, R³ and R⁴ are hydrogen.

In a preferred embodiment,
Y¹ is NR^{5a}, Y² is CR^{5d} and Z is C; or
Y¹ is NR^{5a}, Y² is N and Z is C; or
Y¹ is S, Y² is CR^{5d} and Z is C; or
Y¹ is O, Y² is N and Z is C; or
Y¹ is N, Y² is CR^{5d} and Z is N; or
Y¹ is S, Y² is N and Z is C; or
Y¹ is CR^{5b}, Y² is NR^{5c} and Z is C; or
Y¹ is CR^{5b}, Y² is S and Z is C; or
Y¹ is CR^{5b}, Y² is CR^{5d} and Z is N; or
Y¹ is N, Y² is NR^{5c} and Z is C; or
Y¹ is N, Y² is O and Z is C; or
Y¹ is N, Y² is N and Z is N; or
Y¹ is N, Y² is S and Z is C; or
Y¹ is CR^{5b}, Y² is O and Z is C.

In particular,
Y¹ is NR^{5a}, Y² is CR^{5d} and Z is C; or
Y¹ is NR^{5a}, Y² is N and Z is C; or
Y¹ is S, Y² is CR^{5d} and Z is C.

Preferably, R^{5a}, R^{5b}, R^{5c} and R^{5d}, independently of each other, are selected from the group consisting of hydrogen and C₁-C₄-alkyl. In particular, R^{5a} and R^{5c}, independently of each other, are hydrogen or C₁-C₄-alkyl and R^{5b} and R^{5d} are hydrogen.

R⁶ is preferably selected from the group consisting of hydrogen, C₁-C₄-alkyl, C₃-C₄-alkenyl and phenyl which carries a substituent R¹⁸; where R¹⁸ has one of the above general or, in particular, one of the below preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

In one preferred embodiment R⁶ is hydrogen. In another preferred embodiment R⁶ is C₃-C₄-alkenyl or phenyl which carries a substituent R¹⁸; where R¹⁸ has one of the above general or, in particular, one of the above preferred meanings. Preferably, in this context R¹⁸ is selected from the group consisting of halogen, C₃-C₆-cycloalkyl, C₁-C₄-alkoxy, C₁-C₄-haloalkoxy, C₁-C₄-alkylthio, C₁-C₄-haloalkylthio, C₁-C₄-alkylsulfonyl, C₁-C₄-haloalkylsulfonyl, and C₁-C₄-alkylcarbonyl; and is specifically C₁-C₄-alkylthio, C₁-C₄-haloalkylthio, or C₁-C₄-alkylcarbonyl.

In particular, R⁶ is hydrogen.

Preferably, L¹ is C₁-C₆-alkylene which may carry one or more, in particular 1 or 2, substituents R⁷; where R⁷ has one of the above general or, in particular, one of the below preferred meanings. Preferably, however, each R⁷ in this context is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸, where R¹⁸ has one of the above general or, in particular, one of the below preferred meanings; or two radicals R⁷ bound on the same carbon atom of the alkylene group, form together a group ═O. Preferably, each R¹⁸ in this context is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl; or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo. More preferably, each R¹⁸ in this context is independently selected from the group consisting of halogen, CN, C₁-C₄-alkyl, C₁-C₆-haloalkyl, C₁-C₆-alkoxy and C₁-C₆-haloalkoxy. More preferably, each R⁷ in this context is independently C₁-C₄-alkyl and is specifically methyl.

More preferably, L¹ is CH₂, CH(CH₃) or CH₂CH₂. Specifically, L¹ is CH₂ or CH(CH₃). Very specifically, L¹ is CH₂.

Preferably L² is a bond, C₁-C₆-alkylene or C₁-C₆-alkylene-NR¹⁵, where the alkylene moiety in the two last-mentioned radicals may carry one or more substituents R⁷, where R⁷ and R¹⁵ have one of the above general or, in particular, one of the below preferred meanings. Preferably, however, each R⁷ in this context is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸; or two radicals R⁷ bound on the same carbon atom of the alkylene group, form together a group ═O. Preferably, each R¹⁸ in this context is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl; or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo. More preferably, each R¹⁸ in this context is independently selected from the group consisting of halogen, CN, C₁-C₄-alkyl, C₁-C₆-haloalkyl, C₁-C₆-alkoxy and C₁-C₆-haloalkoxy. More preferably, each R⁷ in this context is independently C₁-C₄-alkyl and is specifically methyl. Also preferably in this context, R¹⁵ is selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl; and is more preferably hydrogen or C₁-C₆-alkyl.

More preferably, L² is a bond, CH₂, CH₂CH₂ or CH₂CH₂NH, and is in particular a bond or CH₂CH₂NH. Specifically, L² is a bond.

A is preferably C₅-C₆-cycloalkyl which may carry one or two substituents R⁹, or is a 5- or 6-membered saturated, partially unsaturated or aromatic heterocyclic ring containing 1 or 2 heteroatoms selected from the group consisting of O, N and S as ring members, where the heterocyclic ring may carry one or more substituents R¹⁰; where R⁹ and R¹⁰ have one of the above general or, in particular, one of the below preferred meanings.

Preferably, however,
each R⁹ in this context is independently selected from the group consisting of halogen, C₁-C₆-alkyl which may carry one or more substituents R¹¹, and C₁-C₆-haloalkyl,
or two radicals R⁹ bound on adjacent ring atoms, together with the ring atoms they are bound to, may form a maximally unsaturated 5- or 6-membered carbocyclic ring;
or two radicals R⁹ bound on non-adjacent ring atoms may form a bridge —CH₂—;
and
each R¹⁰ in this context is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
   where
   each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
   each R¹³ is independently C₁-C₆-alkyl or C₁-C₆-haloalkyl;
   R¹⁴ is phenyl which may carry one or more substituents R¹⁸;
   R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
   or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁷ is independently C₁-C₆-alkyl or C₁-C₆-haloalkyl;
   each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
   or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; and
   R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

More preferably, A is a 5- or 6-membered saturated or aromatic heterocyclic ring containing 1 or 2 heteroatoms selected from the group consisting of O, N and S as ring members, where the heterocyclic ring may carry one or more substituents R¹⁰; where R¹⁰ has one of the above general or, in particular, one of the above or below preferred meanings.

Preferably, however,
each R¹⁰ in this context is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
   where
   each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
   each R¹³ is independently C₁-C₆-alkyl or C₁-C₆-haloalkyl;
   R¹⁴ is phenyl which may carry one or more substituents R¹⁸;
   R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
   or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁷ is independently C₁-C₆-alkyl or C₁-C₆-haloalkyl;
   each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
   or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
   each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; and
   R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

Even more preferably, A is a 5-membered heteroaromatic ring containing one nitrogen atom and one further heteroatom selected from the group consisting of O, N and S as ring members (i.e. A is an oxazole, isoxazole, pyrazole, imidazole, thiazole or isothiazole ring), where the heterocyclic ring may carry one or more substituents R¹⁰; where R¹⁰ has one of the above general or, in particular, one of the above or below preferred meanings.

Preferably, however,
each R¹⁰ in this context is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy; where
   each R¹¹ is independently selected from the group consisting of OH, C₁-C₄-alkoxy, C₁-C₄-haloalkoxy, NR¹⁵R¹⁶ and C(O)NR¹⁵R¹⁶;
   R¹³ is C₁-C₄-alkyl;
   R¹⁵ and R¹⁶, independently of each other and independently of each occurrence, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₈-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
   R²³ and R²⁴, independently of each other and independently of each occurrence, are selected from the group consisting of hydrogen and C₁-C₄-alkylcarbonyl.

In one specific embodiment of the invention, A is selected from the group consisting of oxazolyl, thiazolyl and imidazolyl, in particular from oxazol-2-yl, thiazol-2-yl and imidazol-2-yl, where oxazolyl, thiazolyl, imidazolyl and in particular oxazol-2-yl, thiazol-2-yl and imidazol-2-yl may carry one or two substituents R¹⁰, where R¹⁰ has one of the above general or, in particular, one of the above or below preferred meanings.

Preferably, however,
each R¹⁰ is independently selected from the group consisting of CN, C₁-C₄-alkyl which may carry one or more substituents R¹¹, C₁-C₄-haloalkyl, C(O)R¹⁷, C(O)OR³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy; wherein
   each R¹¹ is independently selected from the group consisting of OH, C₁-C₄-alkoxy, C₁-C₄-haloalkoxy and NR¹⁵R¹⁶;
   R¹³ is C₁-C₄-alkyl;
   R¹⁵ and R¹⁶, independently of each other, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
   R²³ and R²⁴, independently of each other and independently of each occurrence, are selected from the group consisting of hydrogen and C₁-C₄-alkylcarbonyl.

In another specific embodiment of the invention, A is a 5-membered heteroaromatic ring containing one nitrogen atom and one further heteroatom selected from the group consisting of N and S as ring members (i.e. A is a pyrazole, imidazole, thiazole or isothiazole ring), where the heterocyclic ring may carry one or more substituents R¹⁰; where R¹⁰ has one of the above general or, in particular, one of the above or below preferred meanings.

Preferably, however,
each R¹⁰ is independently selected from the group consisting of CN, C₁-C₄-alkyl which may carry one or more substituents R¹¹, C₁-C₄-haloalkyl, C(O)R¹⁷, C(O)OR¹³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy; wherein
   each R¹¹ is independently selected from the group consisting of OH, C₁-C₄-alkoxy, C₁-C₄-haloalkoxy and NR¹⁵R¹⁶;
   R¹³ is C₁-C₄-alkyl;
   R¹⁵ and R¹⁶, independently of each other, are selected from the group consisting of hydrogen, C₁-C₄-alkyl and C₁-C₄-alkylcarbonyl;
   R¹⁷ is C₁-C₄-alkyl;
   each R¹⁸ is independently selected from the group consisting of halogen, C₁-C₆-alkyl which may carry one substituent NR²³R²⁴; C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
   or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
   R²³ and R²⁴, independently of each other and independently of each occurrence, are selected from the group consisting of hydrogen and C₁-C₄-alkylcarbonyl.

In this specific embodiment, A is in particular selected from imidazole and thiazole, where imidazole and thiazole may carry one or two substituents R¹⁰; where R¹⁰ has one of the above general or, in particular, one of the above or below preferred meanings.

More specifically, A is a 5-membered heteroaromatic ring containing one nitrogen atom and one further heteroatom selected from the group consisting of N and S as ring members, where the heterocyclic ring may carry one or two, in particular one, substituents R¹⁰; where R¹⁰ is C₁-C₄-alkyl or C₁-C₄-haloalkyl and is in particular C₁-C₄-haloalkyl. Very specifically A is thiazol-2-yl which may carry one or two, in particular one, substituents R¹⁰; where R¹⁰ is C₁-C₄-alkyl or C₁-C₄-haloalkyl and is in particular C₁-C₄-haloalkyl.

In an alternatively preferred embodiment, L²-A forms a group C₁-C₆-alkylene-NR¹⁵R¹⁶; where R¹⁵ and R¹⁶ have one of the above general meanings. Preferably, however, in this context,
R¹⁵ and R¹⁶, independently of each other, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO, as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo.

More preferably, in this context, R¹⁵ and R¹⁶, independently of each other, are selected from the group consisting of hydrogen, C₁-C₄-alkyl and C₁-C₄-alkylcarbonyl and in particular from hydrogen and C₁-C₄-alkyl. Specifically, they are both hydrogen.

In particular, L²-A forms a group CH₂CH₂—NR¹⁵R¹⁶; where R¹⁵ and R¹⁶ have one of the above general or, in particular, one of the above preferred meanings. Preferably, in this context, R¹⁵ and R¹⁶, independently of each other, are selected from the group consisting of hydrogen, C₁-C₄-alkyl and C₁-C₄-alkylcarbonyl and in particular from hydrogen and C₁-C₄-alkyl. Specifically, they are both hydrogen.

In a preferred embodiment, in compounds I

X¹ is CR¹, X² is CR², X³ is CR³ and X⁴ is CR⁴; or

X¹ is N, X² is CR², X³ is CR³ and X⁴ is CR⁴; or

X¹ is CR¹, X² is N, X³ is CR³ and X⁴ is CR⁴; or

X¹ is CR¹, X² is CR², X³ is N and X⁴ is CR⁴; or

X¹ is CR¹, X² is CR², X³ is CR³ and X⁴ is N; or

X¹ is N, X² is CR², X³ is N and X⁴ is CR⁴; or

X¹ is CR¹, X² is N, X³ is CR³ and X⁴ is N;

where in particular X¹ is CR¹, X² is CR², X³ is CR³ and X⁴ is CR⁴;
Y¹ is NR^{5a}, Y² is CR^{5d} and Z is C; or Y¹ is NR^{5a}, Y² is N and Z is C; or Y¹ is S, Y² is CR^{5d} and Z is C; or Y¹ is O, Y² is N and Z is C; or Y¹ is N, Y² is CR^{5d} and Z is N; or Y¹ is S, Y² is N and Z is C; or Y¹ is CR^{5b}, Y² is NR^{5c} and Z is C; or Y¹ is CR^{5b}, Y² is S and Z is C; or Y¹ is CR^{5b}, Y² is CR^{5d} and Z is N; or Y¹ is N, Y² is NR^{5c} and Z is C; or Y¹ is N, Y² is O and Z is C; or Y¹ is N, Y² is N and Z is N; or Y¹ is N, Y² is S and Z is C; or Y¹ is CR^{5b}, Y² is O and Z is C;
L¹ is C₁-C₆-alkylene which may carry one or more substituents R⁷;
L² is a bond, C₁-C₆-alkylene or C₁-C₆-alkylene-NR¹⁵, where the alkylene moiety in the two last-mentioned radicals may carry one or more substituents R⁷;
A is C₅-C₆-cycloalkyl which may carry 1 or two substituents R⁹, or is a 5- or 6-membered saturated, partially unsaturated or aromatic heterocyclic ring containing 1 or 2 heteroatoms selected from the group consisting of O, N and S as ring members, where the heterocyclic ring may carry one or more substituents R¹⁰;
or L²-A forms a group C₁-C₆-alkylene-NR¹⁵R¹⁶;
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸, and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO, as ring members, where the heterocyclic ring may carry one or more substituents R¹⁸;
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy (where R⁴ is in particular hydrogen, F or methyl, more particularly hydrogen or methyl and specifically hydrogen);
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO, as ring members;
R^{5a}, R^{5b}, R^{5c} and R^{5d}, independently of each other, are selected from the group consisting of hydrogen and C₁-C₄-alkyl;
R⁶ is selected from the group consisting of hydrogen, C₁-C₄ alkyl, C₃-C₄-alkenyl and phenyl which carries a substituent R¹⁸;
each R⁷ is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸; or two radicals R⁷ bound on the same carbon atom of the alkylene group, form together a group ═O;
each R⁹ is independently selected from the group consisting of halogen, C₁-C₆-alkyl which may carry one or more substituents R¹¹, and C₁-C₆-haloalkyl,
or two radicals R⁹ bound on adjacent ring atoms, together with the ring atoms they are bound to, may form a maximally unsaturated 5- or 6-membered carbocyclic ring;
or two radicals R⁹ bound on non-adjacent ring atoms may form a bridge —CH₂—;
each R¹⁰ is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
each R¹³ is independently C₁-C₆-alkyl or C₁-C₆-haloalkyl;
R¹⁴ is phenyl which may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently C₁-C₆-alkyl or C₁-C₆-haloalkyl;
each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; and
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

In a more preferred embodiment, in compounds I
X¹ is CR¹;
X² is CR²;
X³ is CR³;
X⁴ is CR⁴;
Y¹ is NR^{5a}, Y² is CR^{5d} and Z is C; or Y¹ is NR^{5a}, Y² is N and Z is C; or Y¹ is S, Y² is CR^{5d} and Z is C;
L¹ is CH₂, CH(CH₃) or CH₂CH₂;
L² is a bond or CH₂CH₂NH;
A is a 5- or 6-membered saturated or aromatic heterocyclic ring containing 1 or 2 heteroatoms selected from the group consisting of O, N and S as ring members, where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ and R⁴, independently of each other, are selected from the group consisting of hydrogen, F, C₁-C₄-alkyl and C₁-C₄-alkoxy (where R⁴ is in particular hydrogen, F or methyl, more particularly hydrogen or methyl and specifically hydrogen);
or R¹ and R², or R² and R³ form together a bridging group —CH₂CH₂CH₂—, —CH₂CH₂CH₂CH₂—, or —O—CH₂—O—;
R^{5a} and R^{5c}, independently of each other, are hydrogen or C₁-C₄-alkyl;
R^{5b} and R^{5d} are hydrogen;
R⁶ is selected from the group consisting of hydrogen, C₁-C₄ alkyl, C₃-C₄-alkenyl and phenyl which carries a substituent R¹⁸;
each R¹⁰ is independently selected from the group consisting of CN, C₁-C₆-alkyl which may carry one or more substituents R¹¹, C₁-C₆-haloalkyl, C₁-C₆-alkoxy, C₁-C₆-haloalkoxy, S(O)₂R¹⁴, C(O)R¹⁷, C(O)OR¹³, C(O)NR¹⁵R¹⁶, aryl which may carry one or more substituents R¹⁸, and a 5- or 6-membered heteroaromatic ring containing 1, 2, 3 or 4 heteroatoms groups selected from the group consisting of O, N and S as ring members, where the heteroaromatic ring may carry one or more substituents R¹⁸;
or two radicals R¹⁰ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, C₁-C₆-alkyl which may carry one or more substituents R¹¹, C₁-C₆-haloalkyl, C₁-C₆-alkoxy, C₁-C₆-haloalkoxy, C₁-C₆-alkylsulfonyl, C₁-C₆-haloalkylsulfonyl, and phenyl which may carry one or more substituents selected from the group consisting of halogen, C₁-C₆-alkyl, C₁-C₆-haloalkyl, C₁-C₆-alkoxy and C₁-C₆-haloalkoxy;
each R¹¹ is independently selected from the group consisting of OH, C₁-C₆-alkoxy, C₁-C₆-haloalkoxy, NR¹⁵R¹⁶, C(O)OR¹³, C(O)NR¹⁵R¹⁶, phenyl which may carry one or more substituents R¹⁸, and a 3-, 4-, 5-, 6-, 7- or 8-membered saturated heterocyclic ring containing 1 or 2 heteroatoms or heteroatom-containing groups selected from the group consisting of O, N, S, NO, SO and SO₂ as ring members, where the heterocyclic ring may carry one or more substituents R¹⁸;
each R¹³ is independently C₁-C₆-alkyl or C₁-C₆-haloalkyl;
R¹⁴ is phenyl which may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₆-cycloalkyl, C₃-C₆-halocycloalkyl, C₁-C₆-alkylcarbonyl and C₁-C₆-haloalkylcarbonyl;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently C₁-C₆-alkyl or C₁-C₆-haloalkyl;
each R¹⁸ is independently selected from the group consisting of halogen, CN, nitro, OH, SH, C₁-C₆-alkyl which may carry one or more substituents NR²³R²⁴; C₁-C₆-haloalkyl, C₃-C₈-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl and C₁-C₆-haloalkylcarbonyl;
or two radicals R¹⁸ bound on adjacent ring atoms, together with the ring atoms they are bound to, may form a saturated, partially unsaturated or maximally unsaturated 5- or 6-membered carbocyclic or heterocyclic ring, where the heterocyclic ring contains 1 or 2 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; and
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy.

In an even more preferred embodiment, in compounds I
X¹ is CR¹;
X² is CR²;
X³ is CR³;
X⁴ is CR⁴;
Y¹ is NR^{5a}, Y² is CR^{5d} and Z is C; or Y¹ is NR^{5a}, Y² is N and Z is C; or Y¹ is S, Y² is CR^{5d} and Z is C;
L¹ is CH₂, CH(CH₃) or CH₂CH₂;
L² is a bond or CH₂CH₂NH;
A is a 5-membered heteroaromatic ring containing one nitrogen atom and one further heteroatom selected from the group consisting of O, N and S as ring members (i.e. A is an oxazole, isoxazole, pyrazole, imidazole, thiazole or isothiazole ring), where the heterocyclic ring may carry one or more substituents R¹⁰;
R¹ and R², independently of each other, are selected from the group consisting of hydrogen, halogen, CN, C₁-C₄-alkyl, C₁-C₄-alkoxy and C₁-C₄-haloalkoxy;
R³ and R⁴, independently of each other, are selected from the group consisting of hydrogen, F, C₁-C₄-alkyl and C₁-C₄-alkoxy (where R⁴ is in particular hydrogen, F or methyl, more particularly hydrogen or methyl and specifically hydrogen);
or R¹ and R², or R² and R³ form together a bridging group —CH₂CH₂CH₂—, —CH₂CH₂CH₂CH₂—, or —O—CH₂—O—;
R^{5a} and R^{5c}, independently of each other, are hydrogen or C₁-C₄-alkyl;
R^{5b} and R^{5d} are hydrogen;
R⁶ is selected from the group consisting of hydrogen, C₁-C₄ alkyl, C₃-C₄-alkenyl and phenyl which carries a substituent R¹⁸;
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
R¹³ is C₁-C₄-alkyl;
R¹⁵ and R¹⁶, independently of each other and independently of each occurrence, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₈-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
R²³ and R²⁴, independently of each other and independently of each occurrence, are selected from the group consisting of hydrogen and C₁-C₄-alkylcarbonyl.

In particular, in compounds I
X¹ is CR¹;
X² is CR²;
X³ is CR³;
X⁴ is CR⁴;
Y¹ is NR^{5a}, Y² is CR^{5d} and Z is C; or Y¹ is NR^{5a}, Y² is N and Z is C; or Y¹ is S, Y² is CR^{5d} and Z is C;
L¹ is or CH₂, CH(CH₃) or CH₂CH₂; in particular CH₂ or CH(CH₃); specifically CH₂;
L² is a bond;
A is a 5-membered heteroaromatic ring containing one nitrogen atom and one further heteroatom selected from the group consisting of N and S as ring members, where the heterocyclic ring may carry one or more substituents R¹⁰;
R¹ and R², independently of each other, are selected from the group consisting of hydrogen, F, Cl, CN and C₁-C₄-alkyl;
R³ and R⁴ are hydrogen;
R⁵ is hydrogen;
R⁶ is hydrogen;
each R¹⁰ is independently selected from the group consisting of CN, C₁-C₄-alkyl which may carry one or more substituents R¹¹, C₁-C₄-haloalkyl, C(O)R⁷, C(O)OR³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy and NR¹⁵R¹⁶;
each R¹³ is independently C₁-C₄-alkyl;
R¹⁵ and R¹⁶, independently of each other, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

In particular, the compound of formula I is a compound of formula I.a

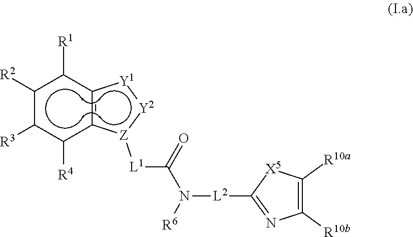

wherein R¹, R², R³, R⁴, R⁶, Y¹, Y², Z, L¹ and L² have one of the above general or, in particular, one of the above preferred meanings; R^{10a} and R^{10b} are independently of each other hydrogen or have one of the general or, in particular, one of the preferred meanings given above for R¹⁰; and X⁵ is S or NR^{x}; where R^{x} is hydrogen or C₁-C₄-alkyl.

Preferably, however, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂, CH(CH₃) or CH₂CH₂;
L² is a bond or CH₂CH₂NH;
X⁵ is S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₂-haloalkoxy; R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R^{5a} is hydrogen or C₁-C₄-alkyl;
R^{5d} is hydrogen;
R⁶ is selected from the group consisting of hydrogen, C₁-C₄-alkyl, C₃-C₄-alkenyl, and phenyl which carries a substituent R¹⁸; where R¹⁸ is as defined in any of the preceding claims;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
R¹¹ is selected from the group consisting of OH and C₁-C₄-alkoxy;
R¹³ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

More preferably, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂, CH(CH₃) or CH₂CH₂; in particular CH₂ or CH(CH₃);
L² is a bond;
X⁵ is S;
R¹ and R², independently of each other, are selected from the group consisting of hydrogen, F, Cl and C₁-C₄-alkyl;
R³ and R⁴ are hydrogen;
R^{5a} is hydrogen or C₁-C₄-alkyl;
R^{5d} is hydrogen;
R⁶ is hydrogen;
R^{10a} is selected from the group consisting of hydrogen, CN, C₁-C₄-alkyl which may carry one substituent R¹¹; and C₁-C₄-haloalkyl; and is in particular selected from the group consisting of hydrogen, C₁-C₄-alkyl and C₁-C₄-haloalkyl;
R^{10b} is selected from the group consisting of hydrogen and phenyl which may carry one or two substituents R¹⁸; and is in particular hydrogen;
or R^{10a} and R^{10b} bound on adjacent ring atoms form together a bridging group —CH═CH—CH═CH—;
each R¹¹ is independently selected from the group consisting of OH and C₁-C₄-alkoxy;
each R¹⁸ is independently selected from the group consisting of halogen, C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members.

Specifically, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂ or CH(CH₃); in particular CH₂;
L² is a bond;
X⁵ is S;
R¹ and R², independently of each other, are selected from the group consisting of hydrogen, F, Cl and methyl; in particular hydrogen, Cl and methyl;
R³ and R⁴ are hydrogen;
R^{5a} is hydrogen or C₁-C₄-alkyl;
R^{5d} is hydrogen;
R⁶ is hydrogen;
R^{10a} is selected from the group consisting of hydrogen and C₁-C₄-haloalkyl; and
R^{10b} is hydrogen.

More specifically, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂;
L² is a bond;
X⁵ is S;
R¹ and R², independently of each other, are selected from the group consisting of hydrogen and methyl;
R³ and R⁴ are hydrogen;
R^{5a} is hydrogen or methyl;
R^{5d} is hydrogen;
R⁶ is hydrogen;
R^{10a} is selected from the group consisting of hydrogen, C₁-C₄-alkyl and C₁-C₄-haloalkyl; in particular hydrogen and C₁-C₄-haloalkyl; and
R^{10b} is hydrogen.

Very specifically, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂;
L² is a bond;
X⁵ is S;
R¹ and R², independently of each other, are selected from the group consisting of hydrogen, Cl and C₁-C₄-alkyl; in particular hydrogen, Cl and methyl;
R³ and R⁴ are hydrogen;
R^{5a} is hydrogen or C₁-C₄-alkyl; in particular hydrogen or methyl;
R^{5d} is hydrogen;
R⁶ is hydrogen;
R^{10a} is selected from the group consisting of hydrogen, C₁-C₄-alkyl and C₁-C₄-haloalkyl; and
R^{10b} is hydrogen.

In an even more specific embodiment, in compounds I.a
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂;
L² is a bond;
X⁵ is S;
R² is selected from the group consisting of hydrogen, Cl and C₁-C₄-alkyl; in particular hydrogen, C; and methyl;
R¹, R³ and R⁴ are hydrogen;
R^{5a} is hydrogen or C₁-C₄-alkyl; in particular hydrogen or methyl;
R^{5d} is hydrogen;
R⁶ is hydrogen;
R^{10a} is C₁-C₄-alkyl or C₁-C₄-haloalkyl; in particular C₁-C₂-alkyl or C₁-C₂-haloalkyl; and
R^{10b} is hydrogen.

In a specific embodiment, the invention relates to a compounds I selected from the compounds of the examples, either in form of free bases or of any pharmaceutically acceptable salt thereof or a stereoisomer, the racemate or any mixture of stereoisomers thereof or a tautomer or a tautomeric mixture or an N-oxide thereof.

The compounds I according to the invention can be prepared by analogy to methods known from the literature and as described in the examples of the present application. In particular, the compounds of the formula I can be prepared according to the following schemes, wherein the variables, if not stated otherwise, are as defined above. An important approach to the compounds according to the invention is the reaction of a carboxylic acid compound 2 with an amine compound 3 to yield the compounds I according to the present invention, as depicted in scheme 1.

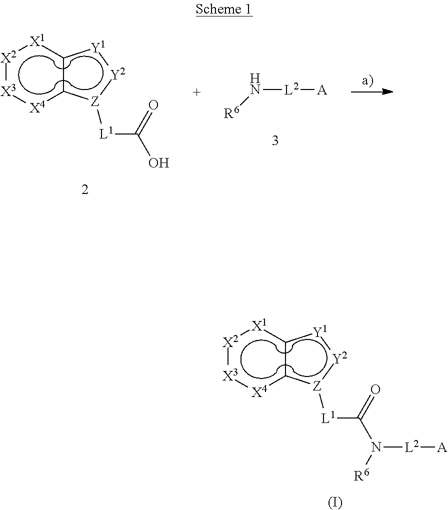

In step a) of scheme 1, the carboxylic acid of the formula 2 reacts with the amine group of compound 3 under conditions suitable for amide bond formation. The skilled person is familiar with the reaction conditions which are required for this type of reaction. Typically, the amide bond formation is carried out in the presence of a coupling reagent. Suitable coupling reagents (activators) are well known and are for instance selected from the group consisting of 1,1′-carbonyldiimidazole (CDI), carbodiimides, such as EDCI (1-ethyl-3-(3-dimethylaminopropyl)carbodiimide; also abbreviated as EDC), DCC (dicyclohexylcarbodiimide) and DIC (diisopropylcarbodiimide), benzotriazole derivatives, such as HOBt (1-hydroxybenzotriazole), HATU (O-(7-azabenzotriazol-1-yl)-N,N,N′,N′-tetramethyluronium hexafluorophosphate), HBTU ((O-benzotriazol-1-yl)-N,N,N′,N′-tetramethyluronium hexafluorophosphate) and HCTU (1H-benzotriazolium-1-[bis(dimethylamino)methylene]-5-chloro tetrafluoroborate), phosphonium-derived activators, such as BOP ((benzotriazol-1-yloxy)-tris(dimethylamino)phosphonium hexafluorophosphate), Py-BOP ((benzotriazol-1-yloxy)-tripyrrolidinphosphonium hexafluorophosphate) and Py-BrOP (bromotripyrrolidinphosphonium hexafluorophosphate), and others, such as COMU ((1-cyano-2-ethoxy-2-oxoethylidenaminooxy)dimethylamino-morpholino-carbenium-hexafluorophosphate). The above activators can also be used in combination with each other. Generally, the activator is used in at least equimolar amounts, with respect to that reactant not used in excess. The benzotriazole and phosphonium coupling reagents are generally used in a basic medium.

Alternatively, the carboxylic acid 2 can be first converted into a so-called active ester, which is obtained in a formal sense by the reaction of the carboxylic acid with an active ester-forming alcohol, such as p-nitrophenol,N-hydroxybenzotriazole (HOBt), N-hydroxysuccinimide or OPfp (pentafluorophenol). The active ester is then reacted with the amine 3 either in the presence or the absence of a coupling reagent.

Furthermore, the OH group of the carboxylic acid 2 can also first be converted into a suitable leaving group (LG), such as Cl, Br, I or a sulfonate, such as tosylate, mesylate, triflate or nonaflate, using reaction procedures that are known to the skilled person. The thus activated carboxylic acid 2 is then reacted with the amine 3. In this variant, the amide bond formation is generally carried out in the presence of a base to neutralize the acid formed during the reaction. Typically, organic bases are used for this purpose. Suitable organic bases are for example tertiary amines, e.g. trimethylamine, triethylamine, tripropylamine, ethyldiisopropylamine and the like, or basic N-heterocycles, such as morpholine, pyridine, lutidine, DABCO, DBU or DBN.

In some particular cases it may be necessary to use appropriate protecting groups in order to avoid side reactions with other reactive groups, which may be present in compound 2 and/or compound 3 and may compete in or disturb the reaction. Just by way of example, if one or more of R¹, R², R³, R⁴, R⁷ and R⁸ is or contains a group C(O)OH, NH₂ or OH and this group has a similar or even stronger reactivity than the desired reaction sites, it is expedient to protect these groups before the above-described amidation reaction is carried out. In these cases, additional deprotecting steps may be necessary to remove these protecting groups after amide bond formation. Suitable protecting groups and the methods for protecting and deprotecting different substituents using such suitable protecting groups are well known to those skilled in the art; examples of which may be found in T. Greene and P. Wuts, Protective Groups in Organic Synthesis (3^{rd} ed.), John Wiley & Sons, NY (1999).

In case that L² is not a bond, the compounds I (termed hereinafter compounds I′) can alternatively be prepared by the reaction of a carboxylic acid compound 2 with a precursor amine 4 to yield the intermediate amide 5, as depicted in scheme 2, which is then further reacted with a compound 6 to yield the compound I′, as depicted in scheme 3.

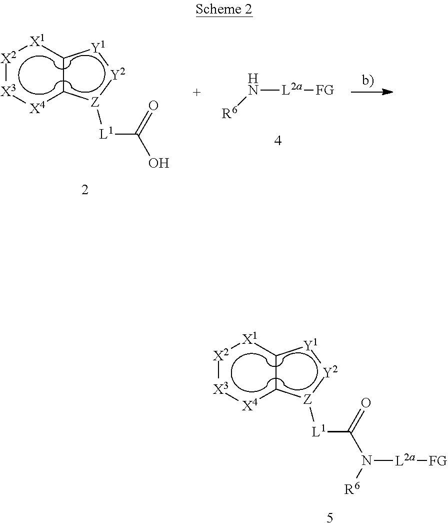

Typically, the amide bond formation in step b) of scheme 2 can be performed as described for step a). The intermediate amide compound 5 is then further reacted with a compound 6 to yield the compound I′, as depicted in scheme 3.

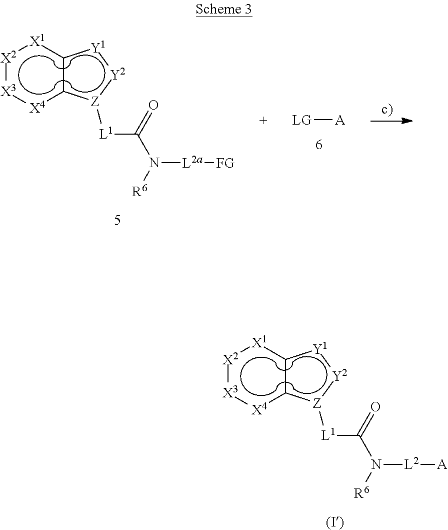

In scheme 3, L² in compound I′ has the aforementioned meanings, but for a bond. L² is selected from C₁-C₆-alkylene which may carry one or more substituents R⁷ and C₃-C₈-cycloalkylene which may carry one or more substituents R⁸. R⁷ and R⁸ are as defined above, under the provision that R⁷ and R⁸ are not selected from functional groups and/or do not comprise any functional groups that might interfere or disturb the reactions in steps b) and c), such as, in particular, halogen, haloalkyl, hydroxyl, CN, SF₅, primary or secondary amines, carboxylic acid or carboxylic acid esters. The choice of suitable R⁷ and R⁸ lies within the routine practice of the skilled person.

The precursor amine 4 carries a suitable functional group (FG) to allow the attachment of further building blocks, in particular to allow the attachment of the cyclic moiety A. For example, FG is selected from —OH, —SH and —N(R¹⁵)H, which may be protected with suitable protective groups, if required, to allow a selective amidation reaction in step b). Before step c), the protective group is of course removed. R¹⁵ is as defined above, under the provision that R¹⁵ is not selected from functional groups and/or does not comprise any functional groups that might interfere or disturb the reactions in steps b) and c). If in the reaction of compounds 4 (and downstream of compounds 5) FG is selected from —OH, —SH and —N(R¹⁵)H, this results in compounds I′ in which L² is C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸.

The compounds 6 comprise the group LG, which, in case that FG is —OH, —SH and —N(R¹⁵)H, is suitably a leaving group, such as those as defined above.

If FG is selected from —OH, —SH and —N(R¹⁵)H, the reaction in step c) is performed under conditions suitable for nucleophilic substitution reactions. Typically, this reaction is performed in the presence of a base. The skilled person is familiar with the reaction conditions which are required for this type of nucleophilic substitution reaction. In case that A is an aromatic or heteroaromatic ring, the exchange of substituents by nucleophilic reagents is however distinctly more difficult than in case of A being a saturated or partially unsaturated ring. It is essential that the leaving group LG in A forms an anion of low energy or an uncharged molecule or can be removed by an energetically advantageous process. Therefore, the leaving group LG is mostly a halide, a sulfonic acid group or a diazonium group in non-activated (hetero)aromatic compounds. Nucleophilic aromatic substitution on carboaromatic rings (phenyl, naphthyl etc.) is eased if the aromatic ring is activated, i.e. contains substituents with a −M effect in ortho and/or para position to the carbon atom carrying the leaving group. Substituents with a −M effect and which fall under the present substituents R¹⁰ are for example the nitro, cyano, formyl, or acetyl group. In this case, also less favoured leaving groups can react; e.g. even hydrogen atoms can be replaced (i.e. LG in 6 can in this case even be H). Electron-poor heteroaromatic rings, like the 6-membered heteroaromatic compounds (pyridine, pyridazine, pyrimidine, pyrazine, the triazines) or quinoline, also undergo readily nucleophilic substitution, even with poor leaving groups, like the hydrogen atom.

In case the group FG in compound 5 is selected from —OH or —N(R¹⁵)H and A is an aromatic or heteroaromatic ring, the reaction in step c) can also be performed under conditions of transition metal-catalyzed C—O or C—N coupling reactions. Transition metal-catalyst C—O or C—N coupling reactions are well known to the skilled person. An important example is the Buchwald-Hartwig reaction. The Buchwald-Hartwig reaction is a transition metal-catalyzed, mostly a Pd catalyzed, C—N or C—O bond formation between an aryl or heteroaryl halogenide or sulfonate and a primary or secondary amine (for C—N bond formation) or an alcohol (for C—O bond formation), generally in the presence of a base. The skilled person is familiar with identifying suitable reaction conditions for the Buchwald-Hartwig reaction.

For preparing compounds I′ in which L² is C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸, it is alternatively possible to use compounds 5 in which FG is a leaving group, such as a halide atom (especially Cl, Br or I or a sulfonate (such as tosylate, mesylate, triflate or nonaflate), and compounds 6 in which LG is a group —OH, —SH or —N(R¹⁵)H. This reaction can be carried out under typical conditions for nucleophilic substitution.

Compounds of the formula 3 can either be purchased or can be readily synthesized using standard methods of heterocyclic chemistry, as for example described in Joule, J. A. and Mills, K. Heterocyclic Chemistry, 5th Edition. 2010, Wiley, Weinheim. ISBN: 978-1-4051-3300-5 and knowledge of functional group interconversion, as for example described in Larock, R. C. Comprehensive Organic Transformations, A Guide to Functional Group Preparations. 2017, Wiley, Weinheim. ISBN: 978-0-470-92795-3.

The compounds of formula 3 can also be synthesized, e.g., following the procedure as depicted in scheme 4.

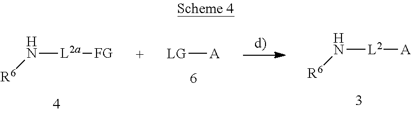

In scheme 4, L² in compound 3 has the aforementioned meanings, but for a bond. L², FG and LG have the aforementioned meanings.

Typically, the reaction in step d) of scheme 4 is performed under conditions suitable for nucleophilic substitution reactions, as described for step c).

For obtaining compounds 3 in which L² is a bond, a compound N(R⁶)H₂ can be used instead of compound 4 for the reaction with 6 in scheme 4.

Several compounds of the formula 2 are commercially available. Those which are not commercially available can be synthesized following different procedures that are described in the prior art, e.g. in Joule, J. A. and Mills, K. Heterocyclic Chemistry, 5th Edition. 2010, Wiley, Weinheim. ISBN: 978-1-4051-3300-5, if necessary using knowledge of functional group interconversion, as for example described in Larock, R. C. Comprehensive Organic Transformations, A Guide to Functional Group Preparations. 2017, Wiley, Weinheim. ISBN: 978-0-470-92795-3. The selection of the appropriate synthetic route depends on the substitution pattern of the compounds of formula 2 and lies within the routine expertise of the skilled person. In the following, the synthesis of some exemplary compounds 2 is specified.

For example, Wittig reaction of N-protected indol-3(2H)-ones or analogous aza systems with suitable ylides and subsequent hydrolysis and, if necessary, deprotection, yields (aza)indole compounds 2a, i.e. compounds 2 in which Y¹ is NR^{5a}, Y² is CR^{5d}, Z is C and L¹ is an optionally substituted methylene bridge, as shown in scheme 5. The reaction can be carried out in analogy to the process described by T. Kawaski et al. in Synthesis, 1991, 701-702. R^{5aa} in compounds 7 is R^{5a}, but for hydrogen, or is a suitable N-protective group, such as acetyl, boc or benzyl. R^{7a} in compounds 8 and 2a is hydrogen or R⁷, as far as it does not disturb the Wittig reaction. Generally it is H or C₁-C₆-alkyl. X is C₁-C₄-alkoxycarbonyl or CN. Hydrolysis of the C₁-C₄-alkoxycarbonyl or CN the direct Wittig product yields the carboxyl group of 2a.

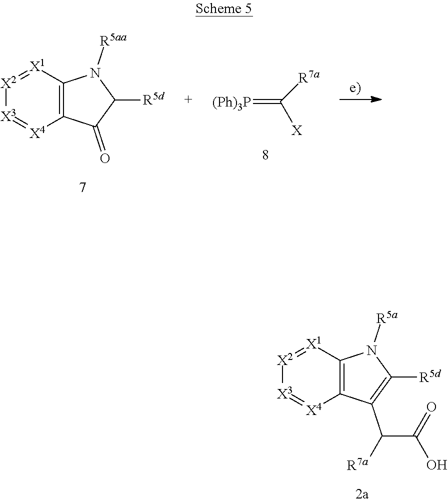

In analogy to the above Wittig reaction, principally all compounds 2 in which Y² is CR^{5d}, Z is C and L¹ is CHR^{7a} can be prepared.

Alternatively, compounds 2 in which Y¹ is NH, Y² is CH, Z is C and L¹ is a methylene bridge, termed in the following compounds 2aa, can be prepared in analogy to the reaction described by K. Samizu et al. in Synlett, 1994, 499-500, as shown in scheme 6 below. Heck reaction of the iodine compound 9 with 2,5-dihydro-2,5-dimethoxyfuran 10 in the presence of a Pd catalyst and a base yields 11. Stirring of 11 with trifluoroacetic acids yields the (aza)indole 12, which can then be hydrolyzed/deprotected to 2aa. R in compounds 9, 11 and 12 is C₁-C₄-alkyl.

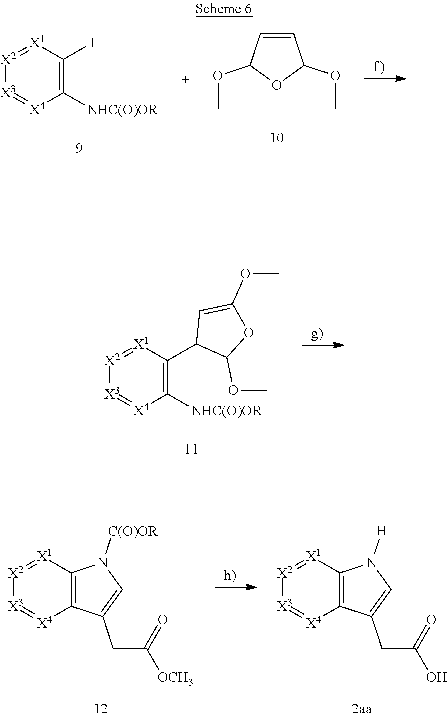

In an alternative route for preparing compounds 2 in which Y¹ is NH, Y² is CR^{5d}, Z is C and L¹ is a methylene bridge (termed hereinafter compounds 2ab), an N-protected indoxyl or its aza derivative is reacted with cyanoacetic acid in a Knoevenagel reaction, in analogy to the synthetic path described by C. Nenitzescu et al. in Chemische Berichte 1958, 1141-1145, and as shown in scheme 7 below. Compound 7, in which R^{5aa} is a protective group, especially an alkylcarbonyl group or boc, is reacted with cyanoacetic acid 13 to 14. Subsequent hydrolysis and if necessary deprotection at the nitrogen atom yields 2ab.

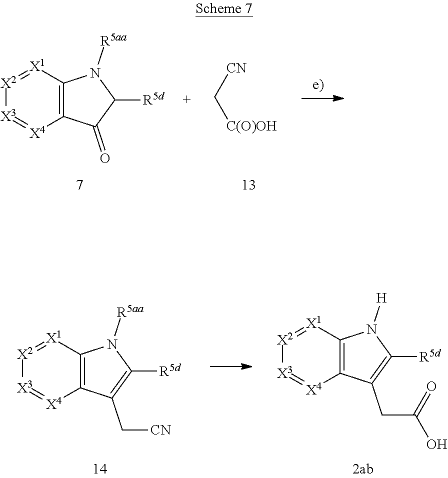

In analogy to the above reaction path of Knoevenagel reaction with cyanoacetic acid and subsequent hydrolysis, principally all compounds 2 in which Y² is CR^{5d}, Z is C and L¹ is CH₂ can be prepared.

Compounds 2 wherein Y¹ is NR^{5a}, Y² is CR^{5d}, Z is C and L¹ is CH₂ (hereinafter termed compounds 2ac) can be obtained by Pd catalyzed alkylation of 15, as described in scheme 8. X is Cl, Br, I or a sulfonate, such as triflate, meslate, tosylate or nonaflate.

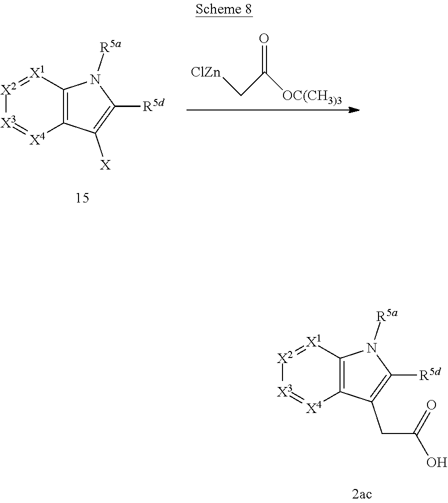

Also possible is the direct acylation of 16 with oxalyl chloride at the 3-position of the indole to 17, followed by reduction to 2ac in analogy to the method described in Brogan, J. T. et al ACS Chemical Neuroscience, 3(9), 658-664; 2012 and depicted in scheme 9. X is Cl, Br, I or a sulfonate, such as triflate, meslate, tosylate or nonaflate.

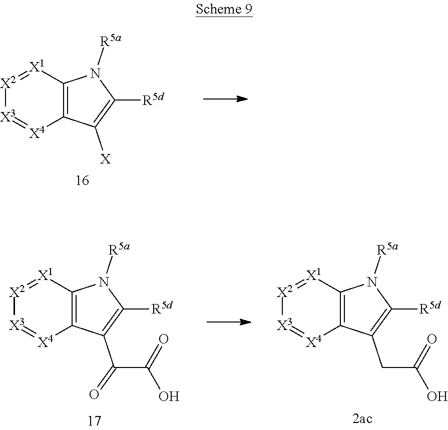

For obtaining compounds 2 in which Y¹ is NR^{5a}, Y² is CR^{5d}, Z is C and L¹ is CH₂CH₂ (hereinafter termed compounds 2b) the aldehyde 18 can be subjected to a Knoevenagel reaction with malonic acid, as shown in scheme 10 below. Double bond hydrogenation, e.g. with Pd catalysis, of 19 yields 2b. 18 in turn can be obtained by Vilsmeier-Hack reaction (for example DMF and POCl₃ followed by hydrolysis) on the indole. R^{5ab} is R⁵ or a protective group.

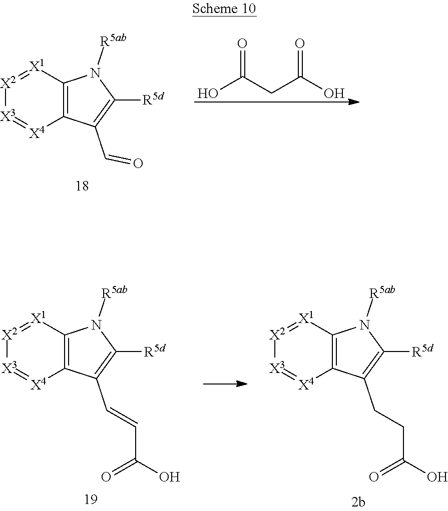

Another method for obtaining compounds 2b is the Heck vinylation of 20 with methylacrylate, as shown in scheme 11 below. R^{5aa} is R^{5a} or a protective group. X is Cl, Br, I or a sulfonate, such as triflate, meslate, tosylate or nonaflate. Double bond hydrogenation, e.g. with Pd catalysis, of 21, ester hydrolysis and, if R^{5aa} is a protective group, deprotection yields 2b.

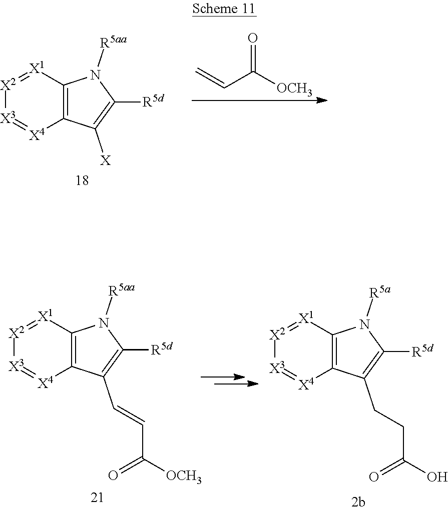

Compounds 2 wherein Y¹ is CR^{5b}, Y² is CR^{5d} and Z is N (termed hereinafter compounds 2c) can be obtained by alkylation or carbonylation of compounds 22, generally in presence of a base such as NaOH, KOH, K₂CO₃, Cs₂CO₃ and the like, in analogy to the method described by Brogan, J. T. et al. ACS Chemical Neuroscience, 3(9), 658-664; 2012, as depicted in scheme 12. LG is Cl or Br. R is C₁-C₄-alkyl.

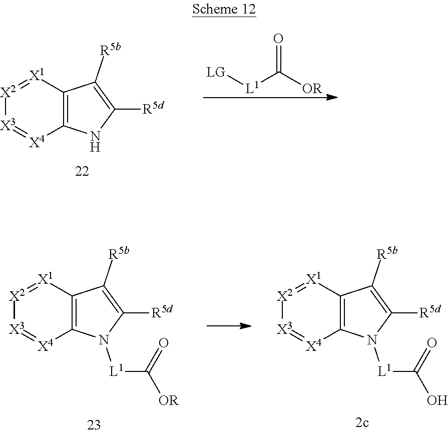

Principally all compounds 2 wherein Z in N can be prepared as depicted in scheme 12.

Indoles used as starting compounds can be prepared using Fischer indole synthesis and variants thereof; Japp-Klingemann indole synthesis; Bartoli indole synthesis; Leimgruber-Batcho indole synthesis; Reissert indole synthesis; and Larock indole synthesis. Azaindoles, i.e. fused systems in which at least one of X¹ to X⁴ is N, are also known. Some specific methods and which often involve ring-closure of an alkynyl or alkenyl group are described in the following papers, and can be modified to produce aza-indoles useful for the current invention: D. K. Whelligan, D. W. Thomson, D. Taylor, S. Hoelder, J. Org. Chem., 2010, 75, 11-15, M. McLaughlin, M. Palucki, I. W. Davies, Org. Lett., 2006, 8, 3307-3310, M. C. de Mattos, S. Alatorre-Santamaria, V. Gotor-Fernández, V. Gotor, Synthesis, 2007, 2149-2152, M. Nazare, C. Schneider, A. Lindenschmidt, D. W. Will, Angew. Chem. Int. Ed., 2004, 43, 4526-4528, H. Schirok, J. Org. Chem., 2006, 71, 5538-5545.

Compounds 2 wherein Y¹ is NH, Y² is N, Z is C and L¹ is CH₂ (termed hereinafter compounds 2da) can be prepared in analogy to the method described in EP-A-0008759 and the literature cited therein and as depicted in scheme 13 below. Aminoacetic acid derivative 24 is reacted under reductive cyclization conditions to 2da. Suitable conditions are the use of metals such as Al, Zn and the like under basic conditions, or the use of hydrazines such as hydrazine, suitably used as hydrate, alkylhydrazines, such as methylhydrazine, hydrazides, such as acethydrazide, or hydrazine salts, such as the hydrochloride. The reaction with a hydrazine compound is generally carried out in the presence of a catalyst, such as activated charcoal or Raney nickel.

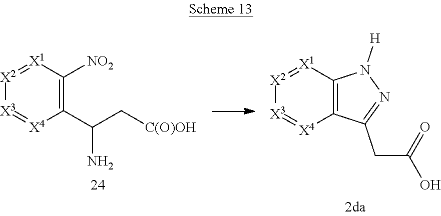

Another approach to compounds 2da is the reaction sequences described by C. Ainsworth in J. Am. Chem. Soc., 1958, 80(4), 967-970 and the literature cited therein and as depicted in scheme 14 below. The 2-carboxyvinyl diazonium chloride 25 is reacted with sodium sulfite to 26, which either reacts directly to 2da under acidic conditions, or is first reduced to 27, e.g. with Zn/HCl, which then reacts to 2da under acidic conditions.

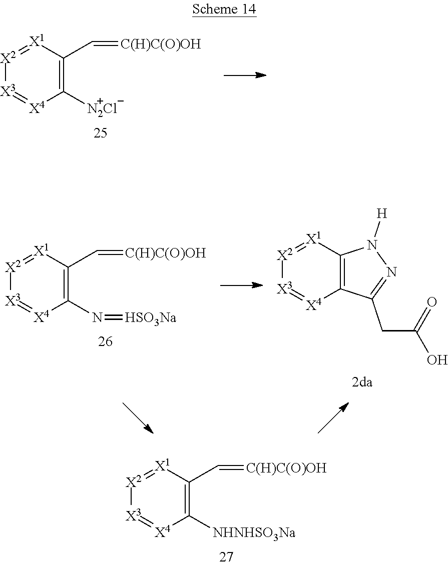

Compounds 2da can furthermore be synthesized in analogy to the process described by N. Halland et al. in Angew. Chem. Int. Ed. 2009, 48, 6879-6882, as depicted in scheme 15 below. The acetylene compound 28, in which X is Cl, Br or I and R is C₁-C₄-alkyl, is reacted with a hydrazine compound 29. In a first step (not shown in scheme 15), X is replaced by a hydrazine radical, followed by an intramolecular hydroamination through a 5-exo-dig cyclization (not shown in scheme 15). Subsequent isomerization gives 30. Hydrolysis of the alkylcarbonyl group then yields 2da.

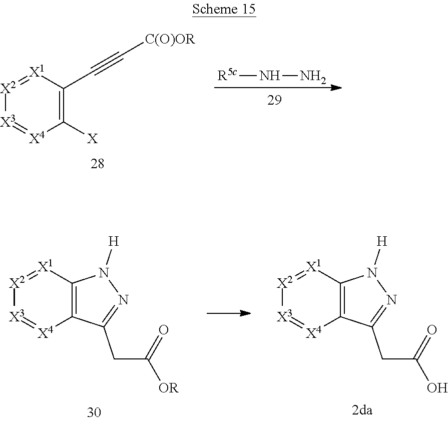

Compounds 2 in which Y¹, Y² and Z are N (termed hereinafter compounds 2e) can be prepared in analogy to the method described by F. Shi in Org. Lett. 2008, 10(12), 2409-2412 by a [3+2] cycloaddition of arynes or derivatives thereof and azides, as shown in scheme 16 below. In compound 31 TMS is trimethylsilyl and OTf is triflate. In situ ortho-elimination in the presence of a fluorine source, such as TBAF or CsF, yields an aryne which reacts with the azide 32, in which R is C₁-C₄-alkyl, in a [3+2] cyclization to 24. Hydrolysis yields 2e.

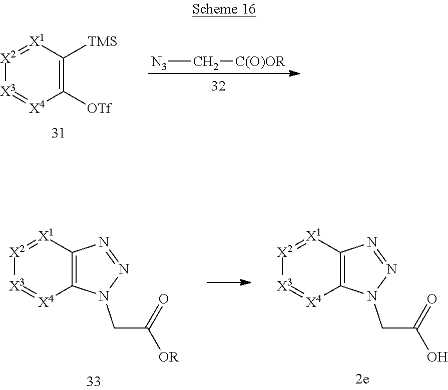

Compounds 2 in which Y¹ is S, Y² is CH Z is C and L¹ is CH₂ (termed hereinafter compounds 2fa) can be prepared in analogy to the method described by N. Beaurain et al. in Journal of Enzyme Inhibition and Medicinal Chemistry 2002, 17(6), 409-414 and as depicted in scheme 17 below. The thiol 34 is reacted with 4-chloro-3-oxobutyric acid ester 35 (R═C₁-C₄-alkyl) to 36. Oxidative ring closure to 37 is effected using suitable oxidizing agents, e.g. phosphorus pentoxide. Finally, the hydrolysis of 37 leads to 2fa.

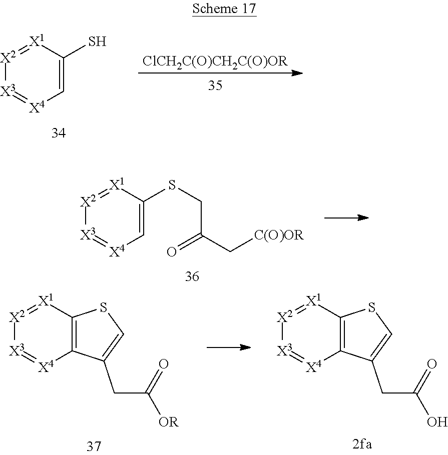

Apart from the method described in scheme 12, compounds 2 wherein Y¹ and Z are N and Y² is CH (hereinafter termed compounds 2ga) can be prepared by the ring closing method described by E. J. Hanan, B. K. Chan, A. A. Estrada, D. G. Shore, J. P. Lyssikatos, Synlett 2010, 2759-2764, as depicted in scheme 18 below. Suitable reaction conditions are Fe/NH₄Cl, isopropanol and formic acid.

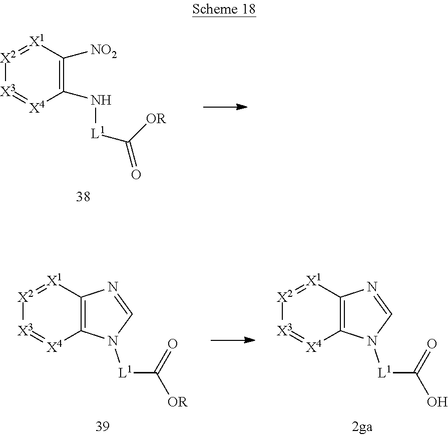

Compounds 2 wherein Y¹ and Z are N and Y² is C—CH₃ (hereinafter termed compounds 2gb) can moreover be prepared by the ring closing method described by S. Caron, B. P. Jones, L. Wei, Synthesis, 2012, 44, 3049-3054 or the method of S. V. Ryabukhin, A. S. Plaskon, D. M. Volochnyuk, A. A. Tolmachev, Synthesis, 2006, 3715-3726, as depicted in scheme 19 below.

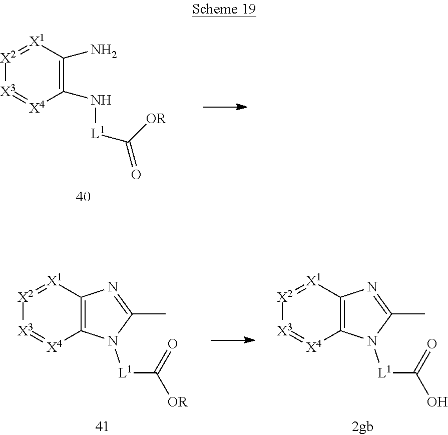

In the method of Caron, 40 is reacted with 2,2,2-trichloroethyl ethanimidate, generally under acidic conditions.

In the method of Ryabukhin, 40 is reacted with trimethylsilyl chloride and oxidized.

Compounds 2 wherein Y¹ and Z are N and Y² is CR^{5d} can moreover be prepared in analogy to the methods described by A. Alonso et al. in Eur. J. Chem. 2011, 234-237. Compounds 2 wherein Y¹ is O, S or NR^{5a}, Y² is N and Z is C can be prepared in analogy to the methods described by M. Gianella et al. in Phytochemistry 1971, 10, 539-544.

Compounds 2 wherein Y¹ is S, Y² is CR^{5d} and Z is N can be prepared in analogy to the methods described by S. Ryabukhin et al. in Synthesis 2006, 21, 3715-3726. Compounds 2 wherein Y¹ is O, Y² is N and Z is C can be prepared in analogy to the methods described by A. Dubrovskiy et al. in Org. Lett. 2010, 12(6), 1180-1183, Dubrovskiy, A. V. et al. ACS Combinatorial Science (2013), 15(4), 193-201, Malik, S. et al. European Journal of Medicinal Chemistry, 84, 42-50; 2014, WO 2008/026217, Yevich, J. P. et al Journal of Medicinal Chemistry, 29(3), 359-69; 1986 or Chauhan, J. et al. Tetrahedron Letters, 53(37), 4951-4954; 2012.

Compounds 2 wherein Y¹ is N, Y² is O or S and Z is C can be prepared in analogy to the methods described by J. P. Yevich et al. in J. Med. Chem. 1986, 29, 359-369 or by M. Jain et al. in J. Med. Chem. 2003, 46, 5428-5436.

Further standard chemical transformation of the introduced functional groups of the above starting materials and intermediates provide further compounds of formula 2.

If not indicated otherwise, the above-described reactions are usually performed in an organic solvent, including aprotic organic solvent, e.g. substituted amides, lactams and ureas; such as dimethylformamide, dimethylacetamide, N-methylpyrrolidone, tetramethyl urea, cyclic ethers; such as dioxane, tetrahydrofurane, halogenated hydrocarbons; such as dichloromethane, and mixtures thereof as well as mixtures thereof with C₁-C₆-alkanols and/or water.

The reactions described above will be usually performed at temperatures between room temperature and the boiling temperature of the solvent employed, depending on the reactivity of the used compounds.

The reaction mixtures are worked up in a conventional way, e.g. by mixing with water, separating the phases and, where appropriate, purifying the crude products by chromatography. If the intermediates and final products are obtained as solids, the purification can also take place by recrystallization or digestion.

Routine experimentations, including appropriate manipulation of the reaction conditions, reagents and sequence of the synthetic route, protection of any chemical functionality that may not be compatible with the reaction conditions, and deprotection at a suitable point in the reaction sequence of the preparation methods are within routine techniques.

Synthesis of the compounds of the invention may be accomplished by methods analogous to those described in the synthetic schemes described hereinabove and in specific examples.

Starting materials, if not commercially available, may be prepared by procedures selected from standard organic chemical techniques, techniques that are analogous to the synthesis of known, structurally similar compounds, or techniques that are analogous to the above described schemes or the procedures described in the synthetic examples section.

The acid addition salts of compounds I are prepared in a customary manner by mixing the free base with a corresponding acid, where appropriate in solution in an organic solvent, for example acetonitrile, a lower alcohol, such as methanol, ethanol or propanol, an ether, such as diethyl ether, methyl tert-butyl ether or diisopropyl ether, a ketone, such as acetone or methyl ethyl ketone, an ester, such as ethyl acetate, mixtures thereof as well as mixtures thereof with water.

The invention further relates to a pharmaceutical composition containing a compound I. The pharmaceutical composition of the invention can contain one or more than one compound of formula I. It comprises moreover at least one pharmaceutically acceptable carrier and/or auxiliary substance.

Examples of suitable carriers and auxiliary substances for the various different forms of pharmaceutical compositions are well known and may be found in the “Handbook of Pharmaceutical Excipients”, 2nd Edition, (1994), Edited by A Wade and P J Weller or in Remington's Pharmaceutical Sciences, Mack Publishing Co. (A. R Gennaro edit. 1985).

For preparing pharmaceutical compositions from the compounds I, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material.

In powders, the carrier is a finely divided solid, which is in a mixture with the finely divided active component. In tablets, the active component is mixed with the carrier having the necessary binding properties in suitable proportions and compacted in the shape and size desired.

The powders and tablets preferably contain from 1% to 80%, more preferably from 5% to 60% of the active compound or active compounds. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. Liquid forms are particularly preferred for topical applications to the eye. For parenteral injection, liquid preparations can be formulated in solution as in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Examples for carriers are thus magnesium carbonate, magnesium stearate, talc, sugar, lactose, pectin, dextrin, starch, gelatin, tragacanth, methylcellulose, sodium carboxymethylcellulose, a low melting wax, cocoa butter, water, water/propylene glycol solutions, or water/polyethylene glycol solutions, and the like.

Examples for auxiliary substances for the present pharmaceutical composition are glidants; wetting agents; emulsifying and suspending agents; dispersants, preservatives; antioxidants; antiirritants; chelating agents; coating auxiliaries; emulsion stabilizers; film formers; gel formers; odor masking agents; flavors, taste corrigents; artificial and natural sweeteners, resin; hydrocolloids; solvents; solubilizers; neutralizing agents; buffers, diffusion accelerators; colorants, pigments; quaternary ammonium compounds; refatting and overfatting agents; raw materials for ointments, creams or oils; silicone derivatives; spreading auxiliaries; stabilizers; sterilants; binders, fillers, disintegrants, coatings; propellants; drying agents; opacifiers; thickeners; waxes; plasticizers, white mineral oils and the like.

The present invention further relates to the compound I as defined above, a stereoisomer, tautomer or pharmaceutically acceptable salt thereof for use as a medicament.

The invention moreover relates to the compound I as defined above, a stereoisomer, tautomer or pharmaceutically acceptable salt thereof for use in the treatment of conditions, disorders or diseases selected from the group consisting of inflammatory diseases, hyperproliferative diseases or disorders, a hypoxia related pathology and a disease characterized by pathophysiological hypervascularization. The invention also relates to the use of compounds I, a stereoisomer, tautomer or pharmaceutically acceptable salt thereof for preparing a medicament for the treatment of conditions, disorders or diseases selected from the group consisting of inflammatory diseases, hyperproliferative diseases or disorders, a hypoxia related pathology and a disease characterized by pathophysiological hypervascularization. The invention also relates to a method for treating conditions, disorders or diseases selected from the group consisting of inflammatory diseases, hyperproliferative diseases or disorders, a hypoxia related pathology and a disease characterized by pathophysiological hypervascularization, which method comprises administering to a patient in need thereof at least one compound I, a stereoisomer, tautomer or pharmaceutically acceptable salt thereof.

In preferred embodiments, the inflammatory disease is selected form the group consisting of atherosclerosis, rheumatoid arthritis, asthma, inflammatory bowel disease, psoriasis, in particular psoriasis vulgaris, psoriasis capitis, psoriasis guttata, psoriasis inversa; neurodermatitis; ichtyosis; alopecia areata; alopecia totalis; alopecia subtotalis; alopecia universalis; alopecia diffusa; atopic dermatitis; lupus erythematodes of the skin; dermatomyositis of the skin; atopic eczema; morphea; scleroderma; alopecia areata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and perioral dermatitis.

In preferred embodiments, the hyperproliferative disease is selected from the group consisting of a tumor or cancer disease, precancerosis, dysplasia, histiocytosis, a vascular proliferative disease and a virus-induced proliferative disease. In particular, the hyperproliferative disease is a tumor or cancer disease selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal (in particular malignant renal cell carcinoma (RCC)), uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.

The precancerosis are for example selected from the group consisting actinic keratosis, cutaneaous horn, actinic cheilitis, tar keratosis, arsenic keratosis, x-ray keratosis, Bowen's disease, bowenoid papulosis, lentigo maligna, lichen sclerosus, and lichen rubber mucosae; precancerosis of the digestive tract, in particular erythroplakia, leukoplakia, Barrett's esophagus, Plummer-Vinson syndrome, crural ulcer, gastropathia hypertrophica gigantea, borderline carcinoma, neoplastic intestinal polyp, rectal polyp, porcelain gallbladder; gynaecological precancerosis, in particular carcinoma ductale in situ (CDIS), cervical intraepithelial neoplasia (CIN), endometrial hyperplasia (grade Ill), vulvar dystrophy, vulvar intraepithelial neoplasia (VIN), hydatidiform mole; urologic precancerosis, in particular bladder papillomatosis, Queyrat's erythroplasia, testicular intraepithelial neoplasia (TIN), carcinoma in situ (CIS); precancerosis caused by chronic inflammation, in particular pyoderma, osteomyelitis, acne conglobata, lupus vulgaris, and fistula.

Dysplasia is frequently a forerunner of cancer, and is can be found in e.g. the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated with the compounds of the present invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephaloophthalmic dysplasia, dysplasia epiphysialis heminelia, dysplasia epiphysialis multiplex, dysplasia epiphysalis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysical dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

A hypoxia related pathology is for example diabetic retinopathy, ischemic reperfusion injury, ischemic myocardial and limb disease, ischemic stroke, sepsis and septic shock (see, e.g. Liu F Q, et al., Exp Cell Res. 2008 Apr. 1; 314(6):1327-36).

A disease characterized by pathophysiological hyper-vascularization is for example angiogenesis in osteosarcoma (see, e.g.: Yang, Qing-cheng et al., Dier Junyi Daxue Xuebao (2008), 29(5), 504-508), macular degeneration, in particular, age-related macular degeneration and vasoproliferative retinopathy (see e.g. Kim J H, et al., J Cell Mol Med. 2008 Jan. 19).

The following examples serve to explain the present invention without limiting its scope.

### EXAMPLES

In the below examples the names of the synthesized target compounds as well as their structure are given. Any discrepancy between name and structure is unintentional; in this case the structure is decisive.

### A. Synthesis Examples

### Examples

The present invention is now illustrated in further details by the following examples, without imposing any limitation thereto.

In the below examples the names of the synthesized target compounds as well as their structure are given. Any discrepancy between name and structure is unintentional; in this case the structure is decisive.

### Abbreviations

aq. for aqueous; DIPEA for N,N-diisopropylethylamine; DMF for dimethylformamide; DMSO for dimethylsulfoxide; EDC for 1-ethyl-3-(3-dimethylaminopropyl)carbodiimide; eq for equivalent; EtOH for ethanol; EtOAc for ethyl acetate; HOAt for 1-hydroxy-7-azabenzotriazole; PyBOP for benzotriazol-1-yl-oxytripyrrolidinophosphonium hexafluorophosphate; r.t. for room temperature; sat. for saturated; sat. for saturated; THF for tetrahydrofuran; TLC for thin layer chromatography.

Compounds can be characterized e.g. by melting point, ¹H-NMR, LC-MS and retention times. ¹H-NMR: The signals are characterized by chemical shift (ppm, δ [delta]) vs. tetramethylsilane, by their multiplicity and by their integral (relative number of hydrogen atoms given). The following abbreviations are used to characterize the multiplicity of the signals: m=multiplet, q=quartet, t=triplet, d=doublet and s=singlet.

### HPLC-MS Instrument Specifications:

Agilent 1100 Series LC/MSD system with DAD¥ELSD and Agilent LC¥MSD VL (G1956A), SL (G1956B) mass-spectrometer or Agilent 1200 Series LC/MSD system with DAD¥ELSD and Agilent LC¥MSD SL (G6130A), SL (G6140A) mass-spectrometer. All the LC/MS data were obtained using positive/negative mode switching.

### Acquisition Parameters:

Column: Zorbax SB-C18 1.8 μm 4.6×15 mm Rapid Resolution cartridge (PN 821975-932); Mobile phase: A—acetonitrile, 0.1% formic acid; B—water (0.1% formic acid); Flow rate: 3 mL/min; Gradient: 0 min—100% B; 0.01 min—100% B; 1.5 min—0% B; 1.8 min—0% B; 1.81 min—100% B; Injection volume: 1 μl; Ionization mode: atmospheric pressure chemical ionization (APCI); Scan range: m/z 80-1000.

### UPLC-MS Specifications

Agilent Infinity 1290 UPLC-MS System; Mass Spectrometer: Single Quadrupole, Electrospray Ionisation; Flow rate: 1 mL/min; inject volume 3 μl; runtime 3 min; Column: Acquity UPLC BEH C18; 1.7 μm; 2.1×50 mm; T=40° C.; Elution: A: Water plus 0.1% trifluoroacetic acid; B: CH₃CN plus 0.1% trifluoroacetic acid; 3 minute gradient: 0 min—5% B; 2.3 min—100% B; 2.5 min—100% B; 2.6 min—5% B; 3 min 5% B.

### HPLC Purification:

Purification was performed using HPLC (H₂O—MeOH, H₂O—CH₃CN; Agilent 1260 Infinity systems equipped with DAD and mass-detectors. Waters Sunfire C18 OBD Prep Column, 100A, 5 μm, 19 mm×100 mm with SunFire C18 Prep Guard Cartridge, 100A, 10 μm, 19 mm×10 mm) The material was dissolved in 0.7 mL DMSO. Flow: 30 mL/min. Purity of the obtained fractions was checked via the analytical LCMS. Spectra were recorded for each fraction as it was obtained straight after chromatography in the solution form. The solvent was evaporated in the flow of N₂ at 80° C. On the basis of post-chromatography LCMS analysis fractions were united. Solid fractions were dissolved in 0.5 mL MeOH/CH₃CN and transferred into a pre-weighted marked vials. Obtained solutions were again evaporated in the flow of N₂ at 80° C. After drying, products were finally characterized by LC-MS and ¹H NMR.

### General Method A

The carboxylic acid in question (2 mmol) and 1,1′-carbonyldiimidazole (2.4 mmol) were dissolved in acetonitrile and stirred for 1 hour. The amine in question (2 mmol) was added to the reaction mixture and refluxed overnight. After TLC control the suspension was cooled and the solvent evaporated under reduced pressure. The residue was treated with water and formed precipitate filtered off, washed with diluted hydrochloric acid, sodium hydrogen carbonate and then again with water. The crude product was purified by recrystallization from isopropyl alcohol. Yield: 40-50%.

### General Method B

The carboxylic acid in question (0.55 mmol), the amine in question (0.50 mmol), 1-hydroxy-7-azabenzotriazole (HOAt, 0.75 mmol) and 1-ethyl-3-(3-dimethylaminopropyl)carbodiimide (EDC, 0.55 mmol) were dissolved in 6 mL of DMF. The resulting slurry was stirred for 24 h at ambient temperature. Thereafter 3 mL of methanol and 0.2 g of silica gel C₁₈ were added sequentially and the mixture was stirred for 2 h, then filtered and solid residue dissolved in 0.5-1 mL of DMSO for HPLC purification (H₂O:MeOH), gradient). Yield: 20-80%.

### Example 1

### N-(5-ethylthiazol-2-yl)-2-(6-methyl-1H-indol-3-yl)acetamide

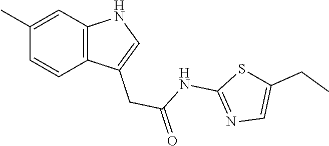 [SMILES: CCc1cnc(NC(=O)Cc2c[nH]c3cc(C)ccc23)s1]

2-(6-Methyl-1H-indol-3-yl)acetic acid (100 mg, 0.53 mmol) was dissolved in DMF (7 mL). 5-Ethylthiazol-2-amine (74.5 mg, 0.58 mmol) and DIPEA (0.18 mL, 0.7 mmol) were added. PyBOP (302.5 mg, 0.58 mmol) was added and the reaction was stirred for 16 h at room temperature. The solvent was removed in vacuo. The residue was dissolved in EtOAc and washed twice with sat. aq. sodium bicarbonate solution, once with water and once with sat. aq. sodium chloride solution. The organic phase was evaporated and the residue was purified by flash chromatography (gradient: 20-100% ethyl acetate in n-heptane). The solvent was evaporated and the title compound was obtained as an orange solid (107 mg, 0.36 mmol, 68% yield). UPLC-MS (Positive mode) m/z 300 (M+H)⁺. Retention time 1.525 min.

### Example 2

### 2-(1H-indol-3-yl)-N-[5-(trifluoromethyl)thiazol-2-yl]acetamide

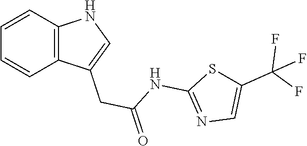 [SMILES: O=C(Cc1c[nH]c2ccccc12)Nc1ncc(C(F)(F)F)s1]

The title compound was prepared according to General Method B using 2-(1H-indol-3-yl)acetic acid and 5-(trifluoromethyl)thiazol-2-amine. Yield 71%. HPLC-MS (Positive mode) m/z 326 (M+H)⁺. Retention time 1.370 min. ¹H NMR (400 MHz, DMSO-d₆): δ=3.92 (s, 2H), 6.99 (t, J=7.0 Hz, 1H), 7.08 (t, J=7.0 Hz, 1H), 7.29 (s, 1H), 7.35 (d, J=7.9 Hz, 1H), 7.55 (d, J=7.0 Hz, 1H), 8.09 (s, 1H), 10.99 (s, 1H), 12.93 (br s, 1H).

### Example 3

### 2-(1-methylindol-3-yl)-N-[5-(trifluoromethyl)thiazol-2-yl]acetamide

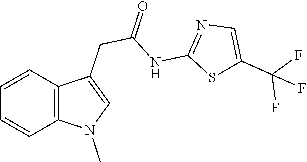

### 3.1 2-(1-methylindol-3-yl)acetic Acid

The title compound was prepared according to procedure reported in Org. Lett., 2010, 12(11), 2660-2663, starting from 2-(1H-indol-3-yl)acetic acid and methyl iodide.

### 3.2 2-(1-methylindol-3-yl)-N-[5-(trifluoromethyl)thiazol-2-yl]acetamide

The title compound was prepared according to General Method B using 2-(1-methylindol-3-yl)acetic acid and 5-(trifluoromethyl)thiazol-2-amine. Yield: 80%. HPLC-MS (Positive mode) m/z 340 (M+H)⁺. Retention time 1.509 min. ¹H NMR (400 MHz, DMSO-d₆): δ=3.76 (s, 3H), 3.92 (s, 2H), 7.03 (t, J=7.5 Hz, 1H), 7.15 (t, J=7.5 Hz, 1H), 7.27 (s, 1H), 7.40 (d, J=7.5 Hz, 1H), 7.57 (d, J=7.5 Hz, 1H), 8.09 (s, 1H), 12.93 (br s, 1H).

### Example 4

### 2-(1-methylindazole-3-yl)-N-[5-(trifluoromethyl)thiazol-2-yl]acetamide

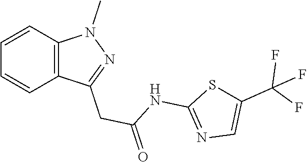 [SMILES: Cn1nc(CC(=O)Nc2ncc(C(F)(F)F)s2)c2ccccc21]

The title compound was prepared according to General Method A using 2-(1-methylindazole-3-yl)acetic acid and 5-(trifluoromethyl)thiazol-2-amine. UPLC-MS (Positive mode) m/z 341 (M+H)⁺. Retention time 1.509 min.

### Example 5

### 2-(benzimidazol-1-yl)-N-(5-methylthiazol-2-yl)acetamide

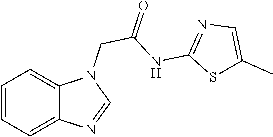

149.2 mg of 2-(benzimidazol-1-yl)acetic acid were dissolved in DMF. 5-Methylthiazol-2-amine (82 mg) and DIPEA (0.5 mL) were added. PyBOP (405 mg) was added and the reaction was allowed to run overnight at room temperature. The solvent was removed in vacuum. The residue was dissolved in EtOAc and washed once with sat. aq. sodium bicarbonate solution, once with water, twice with sat. aq. citric acid, once with brine and dried with sodium sulfate. The organic phase was evaporated and the residue was purified by flash chromatography (n-heptane:EtOAc 1:1). The solvent was removed in vacuum and the desired product was obtained (121 mg). UPLC-MS (Positive mode) m/z 273 (M+H)⁺. Retention time 0.531 min.

### Example 6

### 2-(benzothiophen-3-yl)-N-(5-methylthiazol-2-yl)acetamide

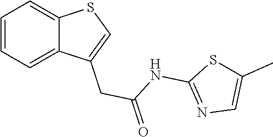 [SMILES: Cc1cnc(NC(=O)Cc2csc3ccccc23)s1]

151.8 mg of 2-(benzothiophen-3-yl)acetic acid were dissolved in DMF. 5-Methylthiazol-2-amine (91 mg) and DIPEA (0.3 mL) were added. PyBOP (450 mg) was added and the reaction was allowed to run overnight at room temperature. The solvent was removed in vacuum. The residue was dissolved in EtOAc and washed once with sat. aq. sodium bicarbonate solution, once with water, twice with sat. aq. citric acid, once with brine and dried with sodium sulfate. The organic phase was evaporated and the residue was purified by flash chromatography (n-heptane:EtOAc 1:1). The solvent was removed in vacuum and the desired product was obtained (16 mg). UPLC-MS (Positive mode) m/z 289 (M+H)⁺. Retention time 1.523 min.

### Example 7

### 2-(benzothiophen-3-yl)-N-[5-(trifluoromethyl)thiazol-2-yl]acetamide

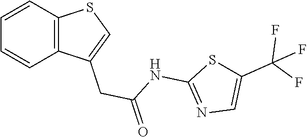 [SMILES: O=C(Cc1csc2ccccc12)Nc1ncc(C(F)(F)F)s1]

The title compound was prepared according to General Method A using 2-(benzothiophen-3-yl)acetic acid and 5-(trifluoromethyl)thiazol-2-amine. Yield: 46%. HPLC-MS (Positive mode) m/z 343 (M+H)⁺. Retention time 1.539 min. ¹H NMR (400 MHz, DMSO-d₆): δ=4.14 (s, 2H), 7.40 (m, 2H), 7.66 (s, 1H), 7.85 (d, J=6.5 Hz, 1H), 7.99 (d, J=7.0 Hz, 1H), 8.12 (s, 1H), 13.07 (s, 1H).

### Example 8

### 2-(benzotriazol-1-yl)-N-[5-(trifluoromethyl)thiazol-2-yl]acetamide

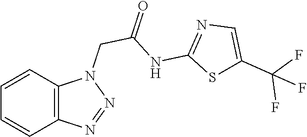 [SMILES: O=C(Cn1nnc2ccccc21)Nc1ncc(C(F)(F)F)s1]

The title compound was prepared according to General Method A using 2-(benzotriazol-1-yl)acetic acid and 5-(trifluoromethyl)thiazol-2-amine. Yield: 44%. HPLC-MS (Positive mode) m/z 328 (M+H)⁺. Retention time 1.326 min. ¹H NMR (400 MHz, DMSO-d₆): δ=5.0 (s, 2H), 7.43 (t, J=8.5 Hz, 1H), 7.58 (t, J=8.5 Hz, 1H), 7.88 (d, J=7.9 Hz, 1H), 8.08 (d, J=8.6 Hz, 1H), 8.17 (s, 1H), 13.40 (s, 1H).

### Example 9

### N-(5-ethylthiazol-2-yl)-2-(4-methylindol-1-yl)acetamide

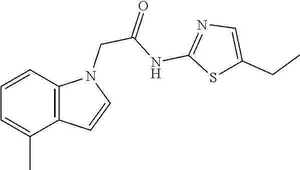 [SMILES: CCc1cnc(NC(=O)Cn2ccc3c(C)cccc32)s1]

2-(4-Methylindol-1-yl)acetic acid (100 mg, 0.53 mmol) was dissolved in DMF (7 mL). 5-Ethylthiazol-2-amine (74.5 mg, 0.58 mmol) and DIPEA (0.18 mL, 0.7 mmol) were added. PyBOP (302.5 mg, 0.58 mmol) was added last and the reaction was allowed to run over night at room temperature. The solvent was removed in vacuo. The residue was dissolved in EtOAc and washed twice with sat. aq. sodium bicarbonate solution, once with water, twice with citric acid, once with water and once with sat. sodium chloride solution. The organic phase was evaporated and the desired product was obtained as a brown solid (152 mg, 0.51 mmol, 96% yield). HPLC-MS (Positive mode) m/z 300 (M+H)⁺. Retention time 1.634 min.

### Example 10

### 2-(6-methyl-1H-indol-3-yl)-N-[5-(trifluoromethyl)thiazol-2-yl]acetamide

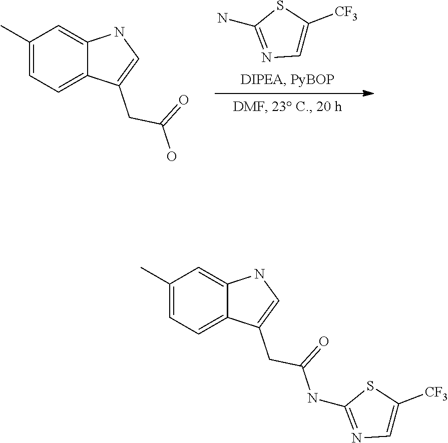 [SMILES: Cc1ccc2c(CC(=O)O)c[nH]c2c1 ; Nc1ncc(C(F)(F)F)s1 ; Cc1ccc2c(CC(=O)Nc3ncc(C(F)(F)F)s3)c[nH]c2c1]

A solution of 2-(6-methyl-1H-indol-3-yl)acetic acid (97.5 mg, 0.52 mmol) and 5 (trifluoromethyl)-thiazol-2-amine (95 mg, 0.57 mmol) in DMF (5 mL) was treated with DIPEA (0.18 mL, 1.03 mmol) and benzotriazol-1-yl-oxytripyrrolidinophosphonium hexafluorophosphate (295 mg, 0.57 mmol), stirred at 23° C. for 20 h and evaporated. The residue was dissolved in EtOAc, washed with a sat NaHCO₃ solution, water and brine and evaporated. The crude was dissolved in DMF (5 mL). HPLC purification (1.0 mL, method A) gave 2-(6-methyl-1H-indol-3-yl)-N-[5-(trifluoromethyl)thiazol-2-yl]acetamide (3.2 mg, 9%) as an off-white solid.

¹H NMR (400 MHz, Chloroform-d) δ=10.00 (br. s, 1H, CO—NH—) 8.13 (br. s, 1H, NH), 7.64 (d, J=1.3 Hz, 1H, H—Ar), 7.43 (d, J=8.1 Hz, 1H, H—Ar), 7.23-7.15 (m, 2H, H—Ar), 7.00 (dd, J=8.1, 1.4 Hz, 1H, H—Ar), 4.01 (s, 2H, CH₂), 2.47 (s, 3H, CH₃) ppm. MS (ESI+, H₂O/MeCN) m/z (%): 340.1 (100, [M+H]⁺).

### Example 11

### 2-(6-chloro-1H-indol-3-yl)-N-[5-(trifluoromethyl)thiazol-2-yl]acetamide

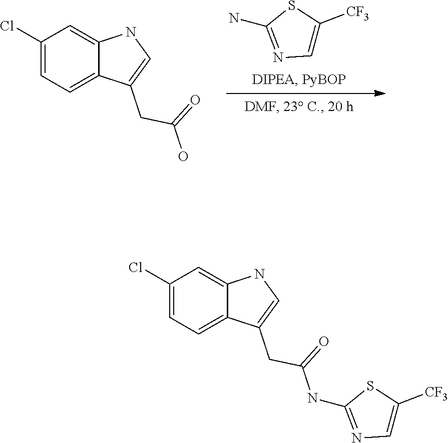 [SMILES: O=C(O)Cc1c[nH]c2cc(Cl)ccc12 ; Nc1ncc(C(F)(F)F)s1 ; O=C(Cc1c[nH]c2cc(Cl)ccc12)Nc1ncc(C(F)(F)F)s1]

A solution of 2-(6-chloro-1H-indol-3-yl)acetic acid (100 mg, 0.48 mmol) and 5-(trifluoromethyl)-thiazol-2-amine (88 mg, 0.53 mmol) in DMF (5 mL) was treated with DIPEA (0.16 mL, 0.95 mmol) and benzotriazol-1-yl-oxytripyrrolidinophosphonium hexafluorophosphate (273 mg, 0.53 mmol), stirred at 23° C. for 20 h and evaporated. The residue was dissolved in EtOAc, the organic phase washed with a sat. citric acid solution, a sat. NaHCO₃ solution, brine and the solvent evaporated. The crude was dissolved in DMF (5 mL). HPLC purification (1.4 mL method A) gave 2-(6-chloro-1H-indol-3-yl)-N-[5-(trifluoromethyl)thiazol-2-yl]acetamide (17.2 mg, 36%) as an off-white solid.

¹H NMR (400 MHz, DMSO-d₆) δ=12.89 (s, 1H, CO—NH—), 11.11-11.05 (m, 1H, NH), 8.04 (d, J=1.6 Hz, 1H, H—Ar), 7.50 (d, J=8.5 Hz, 1H, H—Ar), 7.34 (d, J=1.9 Hz, 1H, H—Ar), 7.28 (d, J=2.4 Hz, 1H, H—Ar), 6.96 (dd, J=8.5, 1.9 Hz, 1H, H—Ar), 3.86 (s, 2H, CH₂) ppm.

MS (ESI+, H₂O/MeCN) m/z (%): 360.0 (100, [M+H]⁺).

### Example 12

### 2-(1,6-dimethylindol-3-yl)-N-(5-methylthiazol-2-yl)acetamide

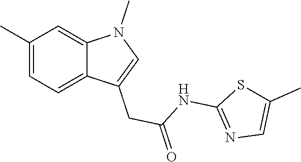

### 12.1 2-(1,6-Dimethyl-1H-indol-3-yl)acetic Acid

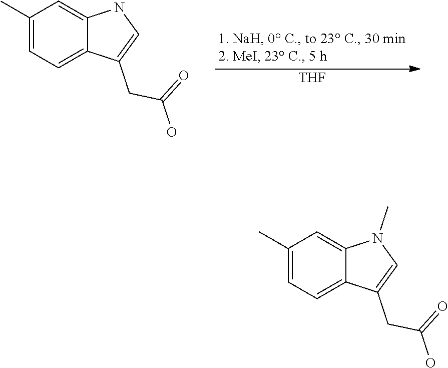

A suspension of sodium hydride (158 mg, 60% dispersion in mineral oil, 3.96 mmol) in anhydrous THF (20 mL) was treated dropwise with a solution of 2-(6-methyl-1H-indol-3-yl)acetic acid (150 mg, 0.79 mmol) in anhydrous THF (5 mL) at 23′ C and stirred at 23° C. for 30 min. The mixture was treated with MeI (163 μL, 2.62 mmol), stirred at 23° C. for 5 h and treated with MeOH (5 mL). The solvent was evaporated, the residue was dissolved in water (50 mL), acidified with 1 M HCl to pH 3 and extracted with CH₂Cl₂ (3×30 mL). The combined organic layers were washed with water (30 mL) and brine (30 mL), dried over anhydrous MgSO₄, filtered and the solvent evaporated. Column chromatography (SiO₂; MeOH/CH₂Cl₂ 0:100->5:95) of the crude gave 2-(1,6-Dimethyl-1H-indol-3-yl)acetic acid (124 mg, 77%) as an off-white solid.

MS (ESI+, H₂O/MeCN) m/z (%): 204.2 (100, [M+H]⁺).

### 12.2 2-(1,6-Dimethyl-1H-indol-3-yl)-N-(5-methylthiazol-2-yl)acetamide

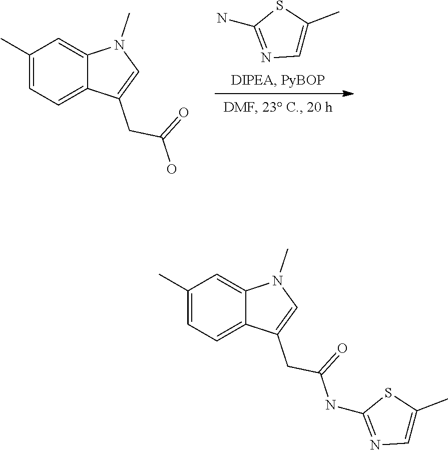

A solution of 2-(1,6-dimethyl-1H-indol-3-yl)acetic acid (100 mg, 0.49 mmol) and 5-methylthiazol-2-amine (61.8 mg, 0.54 mmol) in DMF (5 mL) was treated with DIPEA (0.17 mL, 0.98 mmol) and benzotriazol-1-yl-oxytripyrrolidinophosphonium hexafluorophosphate (282 mg, 0.54 mmol), stirred at 23° C. for 20 h and the solvent evaporated. Recrystallization from EtOAc gave 2-(1,6-Dimethyl-1H-indol-3-yl)-N-(5-methylthiazol-2-yl)acetamide (82 mg, 56%) as a colorless solid.

¹H NMR (400 MHz, DMSO-d₆) δ=12.09 (br. s, 1H, NH), 7.48 (d, J=8.1 Hz, 1H, H—Ar), 7.21-7.09 (m, 3H, H—Ar), 6.87 (dd, J=8.2, 1.4 Hz, 1H, H—Ar), 3.80 (s, 2H, CH₂), 3.71 (s, 3H, N—CH₃), 2.42 (s, 3H, CH₃), 2.31 (s, 3H, CH₃) ppm.

MS (ESI+, H₂O/MeCN) m/z (%): 621.2 (18, [2M+Na]⁺), 599.2 ((16, [2M+H]⁺) 300.2 (100, [M+H]⁺).

### Reference Example 1

Compound of the formula Ref-1 depicted below, which is commercially available, e.g. from Enamine Ltd.

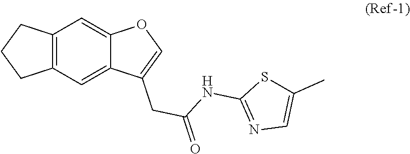 [SMILES: Cc1cnc(NC(=O)Cc2coc3cc4c(cc23)CCC4)s1]

### B. Biological Investigations

### Abbreviations

AUC area under curve
CLL chronic lymphocytic leucemia
DMEM Dulbecco's modified eagle medium
DMSO dimethyl sulfoxide
i.v. or IV intravenous
PBS phosphate buffered saline
PO peroral
QD once a day
Q7D4 4 injections in a 7 days interval
ThPA: N-{[4-(Benzyloxy)phenyl] (methyl)-λ⁴-sulfanylidene}-4-methylbenzenesulfonamide (CAS Number: 21306-65-0; VWR, USA)
Tween 20: polysorbat 20

### General Methods

### Cell Culture

HeLa cells were grown in high-glucose Dulbecco's Modified Eagle's Medium (DMEM, Sigma)+10% FBS+1% penicillin and streptomycin+1% L-glutamine, at 37° C. with 5% CO₂ and 95% humidity. Cytotoxic screening of the ProQinase panel of 100 cell-lines was performed by ProQinase (Freiburg, Germany). Patient derived CLL isolates were prepared and screened as described by Dietrich et al. (S. Dietrich et al., J Clin Invest, 2018, 128(1), 427-445). Cell viability was determined after 48 hours using the ATP-based CellTiter Glo assay (Promega). Luminescence was measured with a Tecan Infinite F200 Microplate Reader (Tecan Group AG) and with an integration time of 0.2 seconds per well.

### Example B.1: Characterization of Compounds for their Influence on egr1 Expression

The compounds of the present invention can be characterized for their effect on expression of egr1 (early growth response protein 1) using an EGR1 reporter cell line.

EGR1 reporter cell lines can be generated, for example, by transfecting cells of a suitable cell line, e.g. HeLa cells, with an expression vector that comprises the coding sequence for at least one reporter, such as luciferase or a GFP (green fluorescent protein), under the control of the EGR1 promoter. This allows for reporter expression to be controlled by stimuli regulating EGR1 transcription (see, for example Gudernova et al., Elife. 6:e21536 (2017)). EGR1 reporter vectors are known in the art and are commercially available (e.g., pGL4[luc2P/hEGR1/Hygro] Vector from Promega Corporation, Madison, Wis., USA, and EGR-1-Luc Reporter Vector from Signosis, Inc., Santa Clara, Calif., USA).

Methods for determining luciferase activity are also well known in the art and generally rely on the measurement of bioluminescent light that is produced in the luciferase-catalyzed conversion of a luciferase substrate (luciferin) by ATP and oxygen in the presence of Mg²⁺ to produce oxyluciferin, AMP, PPᵢ, CO₂ and light. Luciferase assay kits are available, for example, from Promega Corporation, Madison, USA, and Perkin Elmer Inc., Waltham, Mass., USA.

### Generation of a Genomically Engineered EGR1 Reporter HeLa Cell-Line

The HeLa cell line was genetically modified to provide a simple, robust and highly reproducible cell-based assay reporting the activity of an endogenous EGR1 promoter. In brief, a construct encoding EGFP and luciferase proteins, separated by a self-cleaving P2A peptide was inserted, using CRISPR, immediately downstream (3′) to the promoter of endogenous EGR1. Upon treatment with compounds, cells express EGFP and luciferase from EGR1 promoter, which can be readily detected either in live cells using microscopy or cytometry, or through detection of luciferase activity in cell lysates.

To achieve stable genomic integration of an EGR1-promoter dual reporter, two plasmids were generated: one contained the reporter construct (eGFP-P2A-luciferase) flanked by homology arms that direct insertion into genomic DNA, by homologous recombination, of a break in genomic DNA generated by guide RNA targeted cleavage by Cas9 endonuclease. The gRNA expressing plasmid was based on px330, into which a gRNA sequence that targets a break in gDNA close to the start codon of EGR1 was cloned. The left homology arm (encoding part of EGR1 promoter adjacent to its start codon) and right homology arm (encoding upstream of start codon of EGR1) were cloned from gDNA using the following primers:

(SEQ ID NO: 1)
Left HA-rev tcaccatTTGGACGAGCAGGCTGGA
(SEQ ID NO: 2)
Left HA-for gacggccagtgaattCTTCCCCAGCCTAGTTCACG
(SEQ ID NO: 3)
Right HA-rev cgactctagaggatcCCAGTGGCAGAGCCCATTTC
(SEQ ID NO: 4)
Right HA-for tccccgcGGCCAAGGCCGAGATGC

The reporter construct was amplified from HIV-1SDm-CMV-eGFP-P2A-luc plasmid using the following primers:

(SEQ ID NO: 5)
Reporter-for tcgtccaaatggtgagcaagggcgagga
(SEC) ID NO: 6)
Reporter-rev ccttggccgcggggaggcggcccaaagg

The resulting PCR products were cloned into pUC19 vector using an InFusion kit from Clontech. Both vectors were transfected into HeLa cells and suitable derivatives were identified using flow cytometry

### Compound Testing

The present compounds can be tested, e.g. by using a HeLa cell line carrying an EGR1 reporter construct which allows for expression of luciferase and eGFP (enhanced GFP) controlled by the EGR1 promoter. For this reporter cells are seeded in the wells of a 384 well microtiter plate at a density of 2000 cells per well in 48 μl of DMEM supplemented with 4.5 g/l glucose, 2 mM glutamine and 10% FCS and are incubated for 24 hours at 37° C. with 5% CO₂ and 95% humidity. Then, an eleven point 1:3 serial dilution of each test compound, from an initial concentration of 100 μM, is prepared in DMSO and the dilutions are added to the cells in a volume of 2 μl per well. The cells are incubated for a further 24 hours, after which the luciferase activity of each well is determined by addition of 25 μl of luciferase substrate reaction mixture (Britelite™ plus, Perkin Elmer) and measuring the bioluminescence light output (EnVision Xcite plate reader, PerkinElmer). The results are shown in table 1.

The compound of reference example 1 of formula Ref-1 served as a positive control for this EGR1 reporter assay. The compound of example 64 had been identified in an initial high throughput screening campaign. Moreover, massively parallel sequencing of RNA transcripts at multiple time-points from HeLa cells treated with the compound of reference example 1 demonstrated that EGR1 transcripts were upregulated at early time points.

TABLE 1

| Example Number | EC₅₀ |
|---|---|
| 1 | A |
| 2 | B |
| 4 | A |
| 6 | B |
| 10 | A |
| 11 | A |

Key:
A: 10 nM to < 10 μM;
B: 10 μM to < 100 μM;

### Example B.2: Surface Plasmon Resonance

Recombinant human pirin was produced in E. coli with an N-terminal hexahistidine tag and a C-terminal strep tag using a commercially available plasmid construct (pQStrep2-PIR, Addgene Plasmid #31570; Büssow et al, Microbial Cell Factories 4:21 (2005)).

Pirin was covalently linked to a Biacore Series S CM7 chip (GE Healthcare) via amine chemistry in 10 mM acetate buffer, pH 5.5 using 25 μg per ml pirin in the presence of ThPA, a known pirin ligand (Miyazaki et al., Nat. Chem. Biol. 6:667 (2010)) whose presence was included to protect the active site of pirin. A control chip was also prepared under identical condition but without including pirin in the reaction. The sensorgram produced during immobilization demonstrated that pirin was specifically coupled to the surface of the CM7 chip in sufficient amounts to generate a robust signal. A series of increasing concentrations of compound, either the control ThPA or a compound of the present invention is then applied to the pirin modified CM7 chip in phosphate buffered saline containing 2% DMSO and 0.05% tween 20 and sensorgrams are recorded covering the association, equilibrium and dissociation phases of the response.

### Example B.3: Nano Differential Scanning Fluorimetry (NanoDSF)

NanoDSF is an advanced Differential Scanning Fluorimetry method for measuring protein stability using intrinsic tryptophan or tyrosine fluorescence. The fluorescence of the tryptophans and tyrosines in a protein is strongly dependent on their close surroundings. Changes in protein structure typically affect both the intensity and the emission wavelength especially of tryptophan fluorescence. By measuring fluorescence intensity at 330 nm and 350 nm, the change in fluorescence intensity and the shift of the fluorescence maximum upon unfolding can be used to detect thermal melting of the protein. Proteins are stabilized when associated with ligands and show a shift in their melting temperatures. NanoDSF has the advantages of being label free and observing the protein in solution.

A 10 μM solution of pirin in phosphate buffered saline, with or without 20 μM test compound, is subject to thermal denaturation under fluorescence monitoring using a Prometheus NT.48 instrument of NanoTemper Technologies. Unliganded pirin has a complex biphasic melting curve. This may reflect independent melting of the two β-domains within pirin. If the test compound is a ligand to pirin, it adopts a single thermal transition some 20° C. above that of apopirin. This suggests that pirin undergoes substantial structural changes upon binding to the ligands of the present invention.

### Example B.4: In Vitro Test Evaluating Growth Inhibition of Cells Derived from Patients with CLL

The response of 97 tumour samples derived from patients with CLL was investigated. All samples tumor cells were obtained from whole blood, subjected to Ficoll-Isopaque density centrifugation. CD19+ B and CD3+ T cells were isolated by positive magnetic cell separation (Miltenyi Biotec). Sorted cells were checked for purity by fluorescence-activated cell sorting (FACS) with CD19/CD20 for healthy control samples and CD19/CD20/CD5 for CLL samples (BD Biosciences). Following sorting, all samples with a CD19/CD20/CD5 purity <98% were subjected to additional sorting, and the average final purity of all sorted samples was >99%. CLL samples with >100×10⁶ WBC/μL were not subject to purification.

Cells are incubated for three days with an eight-point three-fold titration series of of the test compound from an initial concentration of 30 μM (2000 cells per well in a volume of 50 μl). Cellular viability is estimated by the addition of 25 μL of ATPlite (Perkin Elmer) with the resulting luminescence measured using an EnVision Xcite plate reader (Perkin Elmer).

### Example B.5: In Vivo Test Evaluating the Effects of Test Compounds on the Growth of A549 Cells in Nude Mice

The following test can be conducted for determining, if administration of compounds influences the growth of A549 cells in nude mice, in comparison to solvent only and to carboplatin, a standard of care. An i.p. route of administration is evaluated at 10 and 3 mg/kg delivered i.p., q.d. and compared with solvent control and carboplatin at 75 mg/kg delivered Q7D4 ip. Eight mice are used per study condition.

Compounds are supplied as a dry powder. Each compound is first dissolved in DMSO to yield an appropriate concentration then mixed with 9 volumes of a previously prepared solution of Cremophor-EL: 5% Mannitol (1:8, v/v) warmed to 37° C. while vigorously vortexing. This mixture is sonicated in an ultrasonic bath heated to 40° C. for 15-20 min. The formulations are stable for 24 hours at ambient temperature. A working formulation batch is prepared immediately prior to the in vivo study. A dose volume of 5 ml/kg is used for each concentration and route of administration.

NMRI-nu/nu nude mice are injected subcutaneously in one flank with 5×10⁶ A549 cells in 200 μl of DMEM prepared by trypsinizing an exponentially growing culture of cells. Tumours are allowed to develop to an approximate volume of 100 mm³, (approximately one week after initiation) and thereafter treatment commenced. Body weights and tumour volume are determined every two days. The study lasts for a maximum of a further 28 days, or until the tumour burden exceeded 1000 mm³. At the end of the study, tumours are excised, weighed and then preserved by snap freezing in liquid nitrogen.

### Example B.6: Microsomal Stability

Mouse hepatic microsomes were isolated from pooled (50), perfused livers of Balb/c male mice according to the standard protocol (Hill, J. R. in Current Protocols in Pharmacology 7.8.1-7.8.11, Wiley Interscience, 2003). The batch of microsomes was tested for quality control using Imipramine, Propranolol and Verapamil as reference compounds. Microsomal incubations were carried out in 96-well plates in 5 aliquots of 40 μL each (one for each time point). Liver microsomal incubation medium contained PBS (100 mM, pH 7.4), MgCl₂ (3.3 mM), NADPH (3 mM), glucose-6-phosphate (5.3 mM), glucose-6-phosphate dehydrogenase (0.67 units/ml) with 0.42 mg of liver microsomal protein per ml. Control incubations were performed replacing the NADPH-cofactor system with PBS.

Test compound (2 μM, final solvent concentration 1.6%) is incubated with microsomes at 37° C., shaking at 100 rpm. Incubations are performed in duplicates. Five time points over 40 minutes are analyzed. The reactions are stopped by adding 12 volumes of 90% acetonitrile-water to incubation aliquots, followed by protein sedimentation by centrifuging at 5500 rpm for 3 minutes. Supernatants are analyzed using the HPLC system coupled with tandem mass spectrometer. The elimination constant (kₑₗ), half-life (t½) and intrinsic clearance (Clint) is determined in plot of ln(AUC) versus time, using linear regression analysis.

### Example B.7: Bioavalability

Male Balb/c mice (11-12 weeks old, body weight 23.7 to 30.6 g and average body weight across all groups 26.5 g, SD=1.6 g) are used in this study. The animals are randomly assigned to the treatment groups before the pharmacokinetic study; all animals are fasted for 3 h before dosing. Six time points (IV: 5, 15, 30, 60, 120 and 240 min, and PO: 15, 30, 60, 120, 240, and 360 min) are used in this pharmacokinetic study. Each of the PO and IV time point treatment groups includes 4 animals; there is also control group of 2 animals. Dosing is done according to the treatment schedules outlined in the Table 2. Mice are injected IV with tribrometanol at the dose of 150 mg/kg prior to taking blood. Blood samples are withdrawn from retroorbital sinus and are collected in microcontainers containing K₂EDTA. All samples are immediately prepared, flash-frozen and stored at −70° C. until subsequent bioanalysis.

TABLE 2

| | | | | | Target | |
|---|---|---|---|---|---|---|
| | | | | Target | Dose | Target |
| Number | Test | | | Dose | Concen- | Dose |
| of Mice | com- | Formu- | Delivery | Level | tration | Volume |
| (male) | pound | lation | Route | (mg/kg) | (mg/ml) | (ml/kg) |
| 24 | yes | 1 | PO | 30 | 6 | 5 |
| 24 | yes | 1 | IV | 10 | 2 | 5 |
| 2 | no | 1 | IV | 0 | 0 | 5 |

### Formulation 1: DMSO—Cremophor EL—5% Aqueous Solution of Mannitol

(10%:10%:80%)

Plasma samples (50 μl) are mixed with 200 μl of IS solution (100 ng/ml in acetonitrile-methanol mixture 1:1, v/v). After mixing by pipetting and centrifuging for 4 min at 6,000 rpm, 2 μl of each supernatant is injected into a LC-MS/MS system.

The concentrations of test compound are determined using a high performance liquid chromatography/tandem mass spectrometry (HPLC-MS/MS) method. A Shimadzu HPLC system comprised of 2 isocratic pumps LC-10Advp, an autosampler SIL-HTc, a sub-controller FCV-14AH and a degasser DGU-14A. Mass spectrometric analysis is performed using an API 3000 (triple-quadrupole) instrument from AB Sciex (Canada) with an electro-spray (ESI) interface. The data acquisition and system control is performed using Analyst 1.5.2 software from AB Sciex.

1. A compound of the formula I or a tautomer or a pharmaceutically acceptable salt thereof 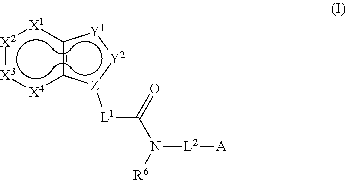
wherein
X¹ is CR¹ or N;
X² is CR² or N;
X³ is CR³ or N;
X⁴ is CR⁴ or N;
with the proviso that at most two of X¹, X², X³ and X⁴ are N;
Y¹ is N, NR^{5a}, S, O or CR^{5b};
Y² is N, NR, S, O or CR^{5d};
Z is N or C;
with the proviso that Y¹ is not O if Y² is CR^{d} and simultaneously Z is C;
with the proviso that Y¹ and Y² are not both simultaneously O or S;
with the proviso that at least one of Y¹, Y² and Z is a heteroatom or heteroatom-containing group;
L¹ is a bond, C₁-C₆-alkylene which may carry one or more substituents R⁷, or C₃-C₈-cycloalkylene which may carry one or more substituents R⁸;
L² is a bond, C₁-C₆-alkylene which may carry one or more substituents R⁷, C₃-C₈-cycloalkylene which may carry one or more substituents R⁸, C₁-C₆-alkylene-O, C₁-C₆-alkylene-S, C₁-C₆-alkylene-NR¹⁵, where the alkylene moiety in the three last-mentioned radicals may carry one or more substituents R⁷; C₃-C₈-cycloalkylene-O, C₃-C₈-cycloalkylene-S or C₃-C₈-cycloalkylene-NR¹⁵, where the cycloalkylene moiety in the three last-mentioned radicals may carry one or more substituents R⁸;
A is 3-, 4-, 5-, 6-, 7- or 8-membered saturated, partially unsaturated or maximally unsaturated carbocyclic ring which may carry one or more substituents R⁹; or a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁰;
or L²-A forms a group C₁-C₆-alkylene-OR¹³, C₁-C₆-alkylene-SR¹⁴ or C₁-C₆-alkylene-NR¹⁵SR¹⁶;
R¹, R², R³ and R⁴, independently of each other, are selected from the group consisting of hydrogen, halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹ and R², or R² and R³, or R³ and R⁴, together with the carbon atoms they are bound to, form a 3-, 4-, 5-, 6- or 7-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members, where the carbocyclic or heterocyclic ring may carry one or more substituents R¹⁸;
R^{5a}, R^{5b}, R^{5c} and R^{5d}, independently of each other, are selected from the group consisting of hydrogen, C₁-C₆-alkyl, C₁-C₆-haloalkyl, aryl, aryl-C₁-C₃-alkyl, where the aryl moiety in the two last-mentioned radicals may carry one or more substituents R¹⁸; hetaryl and hetaryl-C₁-C₃-alkyl, where hetaryl is a 5- or 6-membered heteroaromatic ring containing 1, 2, 3, or 4 heteroatoms selected from the group consisting of O, S and N as ring members, where the heteroaromatic ring may carry one or more substituents R¹⁸;
R⁶ is selected from the group consisting of hydrogen, C₁-C₆-alkyl which may carry one or more substituents R¹¹, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-cycloalkyl-C₁-C₄-alkyl, where cycloalkyl in the two last-mentioned radicals may carry one or more substituents R¹²; C₁-C₆-alkoxy, C₁-C₆-haloalkoxy, aryl, aryl-C₁-C₃-alkyl, where the aryl moiety in the two last-mentioned radicals may carry one or more substituents R¹⁸; heterocyclyl and heterocyclyl-C₁-C₃-alkyl, where heterocyclyl is a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R⁷ and R⁸, independently of each other and independently of each occurrence, are selected from the group consisting of F, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸; or two radicals R⁷ bound on the same carbon atom of the alkylene group, or two radicals R⁸ bound on the same carbon atom of the cycloalkylene group form together a group ═O or ═S;
each R⁹ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on adjacent ring atoms, together with the ring atoms they are bound to, may form a saturated, partially unsaturated or maximally unsaturated 3-, 4, 5- or 6-membered carbocyclic ring which may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R⁹ bound on non-adjacent ring atoms may form a bridge —CH₂— or —(CH₂)₂—;
each R¹⁰ is independently selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or two radicals R¹⁰ bound on adjacent ring atoms, together with the ring atoms they are bound to, may form a saturated, partially unsaturated or maximally unsaturated 3-, 4, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, nitro, SF₅, C₁-C₆-alkyl which may carry one or more substituents R¹¹, C₁-C₆-haloalkyl, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹¹ is independently selected from the group consisting of CN, nitro, SF₅, C₃-C₈-cycloalkyl which may carry one or more substituents R¹², OR¹³, S(O)ₙR¹⁴, NR¹⁵R¹⁶, C(O)R¹⁷, C(O)OR¹³, C(O)NR¹⁵R¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹² is independently selected from the group consisting of halogen, CN, nitro, SF₅, C₁-C₆-alkyl, C₁-C₆-haloalkyl, C₃-C₈-cycloalkyl, C₃-C₈-halocycloalkyl, OR¹³, S(O)ₙR¹⁴, NR¹⁵SR¹⁶, C(O)R¹⁷, C(O)OR¹³, C(O)NR¹⁵SR¹⁶, S(O)₂NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹³ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, S(O)ₘR¹⁴, C(O)R¹⁷, C(O)OR²¹, C(O)NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁴ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, NR¹⁵R¹⁶, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R¹⁵ and R¹⁶, independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, OR²¹, S(O)ₘR²², C(O)R¹⁷, C(O)OR²¹, C(O)NR²³R²⁴, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
or R¹⁵ and R¹⁶, together with the nitrogen atom they are bound to, form a saturated, partially unsaturated or maximally unsaturated 3-, 4-, 5- or 6-membered heterocyclic ring, where the heterocyclic ring may additionally contain 1 or 2 further heteroatoms or heteroatom-containing groups selected from the group consisting of O, N, S, NO, SO and SO₂ as ring members, where the heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁷ is independently selected from the group consisting of hydrogen, C₁-C₆-alkyl which may carry one or more substituents R¹⁹, C₁-C₆-haloalkyl, C₃-C₈-cycloalkyl which may carry one or more substituents R²⁰, aryl which may carry one or more substituents R¹⁸, and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
each R¹⁸ is independently selected from the group consisting of halogen, CN, nitro, OH, SH, SF₅, C₁-C₆-alkyl which may carry one or more substituents selected from the group consisting of CN, OH, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴ and phenyl; C₁-C₆-haloalkyl, C₃-C₈-cycloalkyl which may carry one or more substituents selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl; C₁-C₆-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, carboxyl, C₁-C₆-alkylcarbonyl, C₁-C₆-haloalkylcarbonyl, C₁-C₆-alkoxycarbonyl, C₁-C₆-haloalkoxycarbonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
or two radicals R¹⁸ bound on adjacent ring atoms, together with the ring atoms they are bound to, may form a saturated, partially unsaturated or maximally unsaturated 3-, 4, 5- or 6-membered carbocyclic or heterocyclic ring, where the heterocyclic ring contains 1, 2, 3 or 4 heteroatoms or heteroatom-containing groups selected from the group consisting of O, N, S, NO, SO and SO₂ as ring members, where the carbocyclic or heterocyclic ring may be substituted by one or more radicals selected from the group consisting of halogen, CN, OH, C₁-C₆-alkyl, C₁-C₆-haloalkyl, C₁-C₆-alkoxy, C₁-C₆-haloalkoxy and oxo;
each R¹⁹ is independently selected from the group consisting of CN, OH, C₃-C₈-cycloalkyl, C₃-C₈-halocycloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl, NR²³R²⁴, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
each R²⁰ is independently selected from the group consisting of halogen, CN, OH, C₁-C₆-alkyl, C₁-C₆-haloalkyl, C₁-C₆-alkoxy, C₁-C₆-haloalkoxy, SH, C₁-C₆-alkylthio, C₁-C₆-haloalkylthio, C₁-C₆-alkylsulfonyl, C₁-C₆-haloalkylsulfonyl and phenyl;
R²¹ and R²², independently of each other and independently of each occurrence, are selected from the group consisting of hydrogen, C₁-C₆-alkyl which may carry one or more substituents R¹⁹, C₁-C₆-haloalkyl, C₃-C₈-cycloalkyl, C₃-C₈-halocycloalkyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
R²³ and R²⁴, independently of each other and independently of each occurrence, are selected from the group consisting of hydrogen, C₁-C₆-alkyl, C₁-C₆-haloalkyl, C₃-C₈-cycloalkyl, C₃-C₈-halocycloalkyl, C₁-C₆-alkylcarbonyl, C₁-C₆-haloalkylcarbonyl, C₁-C₆-alkoxycarbonyl, C₁-C₆-haloalkoxycarbonyl, C₁-C₆-alkylsulfonyl, C₁-C₆-haloalkylsulfonyl, aryl and a 3-, 4-, 5-, 6-, 7- or 8-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where aryl or the heterocyclic ring may carry one or more substituents selected from the group consisting of halogen, CN, OH, C₁-C₆-alkyl, C₁-C₆-haloalkyl, C₁-C₆-alkoxy and C₁-C₆-haloalkoxy;
m is 1 or 2; and
n is 0, 1 or 2.

2. The compound as claimed in claim 1, wherein
X¹ is CR¹, X² is CR², X³ is CR³ and X⁴ is CR⁴; or
X¹ is N, X² is CR², X³ is CR³ and X⁴ is CR⁴; or
X¹ is CR¹, X² is N, X³ is CR³ and X⁴ is CR⁴; or
X¹ is CR¹, X² is CR², X³ is N and X⁴ is CR⁴; or
X¹ is CR¹, X² is CR², X³ is CR³ and X⁴ is N; or
X¹ is N, X² is CR², X³ is N and X⁴ is CR⁴; or
X¹ is CR¹, X² is N, X³ is CR³ and X⁴ is N.

3. The compound as claimed in claim 2, wherein X¹ is CR¹, X² is CR², X³ is CR³ and X⁴ is CR⁴.

4. The compound as claimed in claim 1, wherein
R¹ and R², independently of each other, are selected from the group consisting of hydrogen, halogen, CN, C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, C₁-C₆-alkylthio, C₁-C₆-haloalkylthio, phenyl which may carry one or more substituents R¹⁸ and a 5- or 6-membered saturated, partially unsaturated or maximally unsaturated heterocyclic ring containing 1, 2, 3 or 4 heteroatoms or heteroatom-containing groups selected from the group consisting of O, N, S, NO, SO and SO₂ as ring members, where the heterocyclic ring may carry one or more substituents R¹⁸;
R³ and R⁴, independently of each other, are selected from the group consisting of hydrogen, halogen, CN, C₁-C₆-alkyl, C₁-C₆-haloalkyl, C₁-C₄-alkoxy and C₁-C₄-haloalkoxy;
or R¹ and R², or R² and R³, together with the carbon atoms they are bound to, form a 5- or 6-membered saturated, partially unsaturated or maximally unsaturated carbocyclic or heterocyclic ring, where the heterocyclic ring contains 1, 2 or 3 heteroatoms or heteroatom-containing groups selected from the group consisting of O, N, S, NO, SO and SO₂ as ring members.

5. The compound as claimed in claim 4, wherein
R¹ and R², independently of each other, are selected from the group consisting of hydrogen, halogen, CN, C₁-C₄-alkyl, C₁-C₄-alkoxy and C₁-C₄-haloalkoxy;
R³ and R⁴, independently of each other, are selected from the group consisting of hydrogen, F, C₁-C₄-alkyl and C₁-C₄-alkoxy;
or R¹ and R², or R² and R³ form together a bridging group —CH₂CH₂CH₂—, —CH₂CH₂CH₂CH₂—, or —O—CH₂—O—.

6. The compound as claimed in claim 1, wherein
Y¹ is NR^{5a}, Y² is CR^{d} and Z is C; or
Y¹ is NR^{5a}, Y² is N and Z is C; or
Y¹ is S, Y² is CR^{5d} and Z is C; or
Y¹ is O, Y² is N and Z is C; or
Y¹ is N, Y² is CR^{5d} and Z is N; or
Y¹ is S, Y² is N and Z is C; or
Y¹ is CR^{5b}, Y² is NR^{5c} and Z is C; or
Y¹ is CR^{5b}, Y² is S and Z is C; or
Y¹ is CR^{5b}, Y² is CR^{5d} and Z is N; or
Y¹ is N, Y² is NR^{5c} and Z is C; or
Y¹ is N, Y² is O and Z is C; or
Y¹ is N, Y² is N and Z is N; or
Y¹ is N, Y² is S and Z is C; or
Y¹ is CR^{5b}, Y² is O and Z is C.

7. The compound as claimed in claim 6, wherein
Y¹ is NR^{5a}, Y² is CR^{5d} and Z is C; or
Y¹ is NR^{5a}, Y² is N and Z is C; or
Y¹ is S, Y² is CR^{5d} and Z is C.

8. The compound as claimed in claim 1, wherein R^{5a}, R^{5b}, R^{5c} and R^{5d}, independently of each other, are selected from the group consisting of hydrogen and C₁-C₄-alkyl.

9. The compound as claimed in claim 1, wherein
L¹ is C₁-C₆-alkylene which may carry one or more substituents R⁷; and
L² is a bond, C₁-C₆-alkylene or C₁-C₆-alkylene-NR¹⁵, where the alkylene moiety in the two last-mentioned radicals may carry one or more substituents R⁷; where
each R⁷ is independently selected from the group consisting of F, CN, OH, C₁-C₄-alkyl, C₁-C₄-haloalkyl, C₃-C₆-cycloalkyl, C₃-C₆-halocycloalkyl, C₁-C₄-alkoxy, C₁-C₄-haloalkoxy and phenyl which may carry one or more substituents R¹⁸;
or two radicals R⁷ bound on the same carbon atom of the alkylene group, form together a group ═O; and
R¹⁵ and R¹⁸ are as defined in claim 1.

10. The compound as claimed in claim 8, wherein
L¹ is CH₂, CH(CH₃) or CH₂CH₂; and
L² is a bond, CH₂, CH₂CH₂ or CH₂CH₂NH.

11. The compound as claimed in claim 1, wherein R⁶ is hydrogen or C₁-C₄-alkyl.

12. The compound as claimed in claim 1, wherein R⁶ is C₃-C₄-alkenyl or phenyl, where phenyl may carry a substituent R¹⁸; where R¹⁸ is as defined in claim 1.

13. The compound as claimed in claim 1, wherein
A is a 5-membered heteroaromatic ring containing one nitrogen atom and one further heteroatom selected from the group consisting of O, N and S as ring members, where the heterocyclic ring may carry one or more substituents R¹⁰;
where
each R¹⁰ is independently selected from the group consisting of CN, C₁-C₄-alkyl which may carry one or more substituents R¹¹, C₁-C₄-haloalkyl, C(O)R¹⁷, C(O)OR¹³, C(O)NR¹⁵R¹⁶, phenyl which may carry one or more substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH—, —CH₂CH₂CH₂— or —CH₂CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy, NR¹⁵R¹⁶ and C(O)NR¹⁵R¹⁶;
R¹³ is C₁-C₄-alkyl;
R¹⁵ and R¹⁶, independently of each other and independently of each occurrence, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₈-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing 1 or 2 heteroatoms or heteroatom-containing groups selected from the group consisting of O, N, S, NO, SO and SO₂ as ring members, where the heterocyclic ring may be substituted by one or more radicals selected from the group consisting of halogen, C₁-C₄-alkyl, C₁-C₄-haloalkyl, C₁-C₄-alkoxy, C₁-C₄-haloalkoxy and oxo; and
R²³ and R²⁴, independently of each other and independently of each occurrence, are selected from the group consisting of hydrogen and C₁-C₄-alkylcarbonyl.

14. The compound as claimed in claim 13, wherein
A is a 5-membered heteroaromatic ring containing one nitrogen atom and one further heteroatom selected from the group consisting of N and S as ring members, where the heterocyclic ring may carry one or more substituents R¹⁰;
wherein
each R¹⁰ is independently selected from the group consisting of CN, C₁-C₄-alkyl which may carry one or more substituents R¹¹, C₁-C₄-haloalkyl, C(O)R¹⁷, C(O)OR¹³, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or two radicals R¹⁰ bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
each R¹¹ is independently selected from the group consisting of OH, C₁-C₄-alkoxy, C₁-C₄-haloalkoxy and NR¹⁵R¹⁶;
R¹³ is C₁-C₄-alkyl;
R¹⁵ and R¹⁶, independently of each other, are selected from the group consisting of hydrogen, C₁-C₄-alkyl and C₁-C₄-alkylcarbonyl;
R¹⁷ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one nitrogen ring atom or one or two oxygen atoms as ring members, where the heterocyclic ring may be substituted by an oxo group; and
R²³ and R²⁴, independently of each other and independently of each occurrence, are selected from the group consisting of hydrogen and C₁-C₄-alkylcarbonyl.

15. The compound as claimed in claim 13, wherein A is selected from the group consisting of oxazol-2-yl, thiazol-2-yl and imidazol-2-yl, where oxazol-2-yl, thiazol-2-yl and imidazol-2-yl may carry one or two substituents R¹⁰, where R¹⁰ is as defined in claim 1.

16. The compound as claimed in claim 1, wherein the compound of formula I is a compound of formula I.a 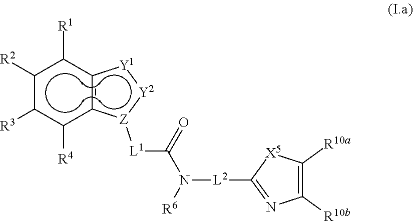
wherein
Y¹ is NR^{5a}, Y² is CR^{5d} and Z is C; or
Y¹ is NR^{5a}, Y² is N and Z is C; or
Y¹ is S, Y² is CR^{5d} and Z is C;
L¹ is CH₂, CH(CH₃) or CH₂CH₂;
L² is a bond or CH₂CH₂NH;
X⁵ is S or NR^{x};
R^{x} is hydrogen or C₁-C₄-alkyl;
R¹ and R², independently of each other, are selected from the group consisting of hydrogen, F, Cl, CN, C₁-C₄-alkyl, C₁-C₂-alkoxy and C₁-C₂-haloalkoxy;
R³ is selected from the group consisting of hydrogen, C₁-C₄-alkyl and C₁-C₄-alkoxy;
or R² and R³ form together a bridging group —CH₂CH₂CH₂— or —O—CH₂—O—;
R⁴ is hydrogen;
R^{5a} is hydrogen or C₁-C₄-alkyl;
R^{5d} is hydrogen;
R⁶ is selected from the group consisting of hydrogen, C₁-C₄-alkyl, C₃-C₄-alkenyl, and phenyl which carries a substituent R¹⁸; where R¹⁸ is as defined in claim 1;
R^{10a} is selected from the group consisting of hydrogen, CN, C₁-C₄-alkyl which may carry one substituent R¹¹; C₁-C₄-haloalkyl, and C(O)OR¹³;
R^{10b} is selected from the group consisting of hydrogen, C₁-C₄-alkyl, phenyl which may carry one or two substituents R¹⁸, and a 5- or 6-membered heteroaromatic ring containing one heteroatom selected from the group consisting of O, N and S as ring members, where the heteroaromatic ring may carry one or more substituents R¹⁸;
or R^{10a} and R^{10b} bound on adjacent ring atoms form together a bridging group —CH═CH—CH═CH— or —CH₂CH₂CH₂—, where one of the hydrogen atoms in the bridging group may be substituted by a radical selected from the group consisting of methyl and methoxy;
R¹¹ is selected from the group consisting of OH and C₁-C₄-alkoxy;
R¹³ is C₁-C₄-alkyl;
each R¹⁸ is independently selected from the group consisting of halogen, C₁-C₆-alkyl which may carry one substituent NR²³R²⁴; C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, NR²³R²⁴, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members; and
R²³ and R²⁴, independently of each other and independently of each occurrence, are selected from the group consisting of hydrogen and C₁-C₄-alkylcarbonyl.

17. The compound as claimed in claim 16, wherein
Y¹ is NR^{5a}, Y² is CR^{d} and Z is C; or
Y¹ is NR^{5a}, Y² is N and Z is C; or
Y¹ is S, Y² is CR^{5d} and Z is C;
L¹ is CH₂, CH(CH₃) or CH₂CH₂;
L² is a bond;
X⁵ is S;
R¹ and R², independently of each other, are selected from the group consisting of hydrogen, F, Cl and C₁-C₄-alkyl;
R³ and R⁴ are hydrogen;
R^{5a} is hydrogen or C₁-C₄-alkyl;
R^{5d} is hydrogen;
R⁶ is hydrogen;
R^{10a} is selected from the group consisting of hydrogen, CN, C₁-C₄-alkyl which may carry one substituent R¹¹; and C₁-C₄-haloalkyl;
R^{10b} is selected from the group consisting of hydrogen and phenyl which may carry one or two substituents R¹⁸;
or R^{10a} and R^{10b} bound on adjacent ring atoms form together a bridging group —CH═CH—CH═CH—;
each R¹¹ is independently selected from the group consisting of OH and C₁-C₄-alkoxy;
each R¹⁸ is independently selected from the group consisting of halogen, C₃-C₆-cycloalkyl, C₁-C₄-alkoxy, C₁-C₆-haloalkoxy, C₁-C₆-alkylthio, C₁-C₆-haloalkylthio, C₁-C₆-alkylsulfonyl, C₁-C₆-haloalkylsulfonyl, and C₁-C₆-alkylcarbonyl;
or two radicals R¹⁸ bound on adjacent ring atoms, together with the ring atoms they are bound to, may form a saturated 5- or 6-membered heterocyclic ring containing one or two oxygen atoms as ring members.

18. The compound as claimed in claim 17, wherein
Y¹ is NR^{5a}, Y² is CR⁵ and Z is C; or
Y¹ is NR^{5a}, Y² is N and Z is C; or
Y¹ is S, Y² is CR^{5d} and Z is C;
L¹ is CH₂;
L² is a bond;
X⁵ is S;
R² is selected from the group consisting of hydrogen, Cl and C₁-C₄-alkyl;
R¹, R³ and R⁴ are hydrogen;
R^{5a} is hydrogen or C₁-C₄-alkyl;
R^{5d} is hydrogen;
R⁶ is hydrogen;
R^{10a} is C₁-C₄-alkyl or C₁-C₄-haloalkyl; and
R^{10b} is hydrogen.

19. The compound of formula I.a 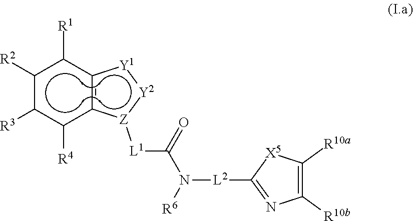
a tautomer, or a pharmaceutically acceptable salts thereof, wherein the variables for a single compound have the meanings given in one line of the following table:

| No. | Y¹—Y²—Z | R¹ | R² | R³ | R⁴ | L¹ | R⁶ | L² | X⁵ | R^{10a} | R^{10b} |
|---|---|---|---|---|---|---|---|---|---|---|---|
| 1 | NH—CH═C | H | CH₃ | H | H | CH₂ | H | bond | S | CH₂CH₃ | H |
| 2 | NH—CH═C | H | H | H | H | CH₂ | H | bond | S | CF₃ | H |
| 3 | N(CH₃)—CH═C | H | H | H | H | CH₂ | H | bond | S | CF₃ | H |
| 4 | N(CH₃)—N═C | H | H | H | H | CH₂ | H | bond | S | CF₃ | H |
| 5 | N═CH—N | H | H | H | H | CH₂ | H | bond | S | CH₃ | H |
| 6 | S—CH═C | H | H | H | H | CH₂ | H | bond | S | CH₃ | H |
| 7 | S—CH═C | H | H | H | H | CH₂ | H | bond | S | CF₃ | H |
| 8 | N═N—N | H | H | H | H | CH₂ | H | bond | S | CF₃ | H |
| 9 | CH═CH—N | CH₃ | H | H | H | CH₂ | H | bond | S | CH₂CH₃ | H |
| 10 | NH—CH═C | H | CH₃ | H | H | CH₂ | H | bond | S | CF₃ | H |
| 11 | NH—CH═C | H | Cl | H | H | CH₂ | H | bond | S | CF₃ | H |
| 12 | N(CH₃)—CH═C | H | CH₃ | H | H | CH₂ | H | bond | S | CH₃ | H |

20. A pharmaceutical composition comprising a compound as defined in claim 1 or a tautomer or a pharmaceutically acceptable salt thereof.

21. (canceled)

22. A method to treat a condition, disorder or disease in a patient in need thereof comprising administering to the patient in need thereof a compound or a tautomer or a pharmaceutically acceptable salt thereof as described in claim 1, wherein the condition, disorder or disease is selected from the group consisting of inflammatory diseases, hyperproliferative diseases or disorders, a hypoxia related pathology and a disease characterized by pathophysiological hypervascularization.

23. The method of claim 22, wherein the conditions, disorders or diseases are selected from the group consisting of atherosclerosis, rheumatoid arthritis, asthma, inflammatory bowel disease, psoriasis psoriasis vulgaris, psoriasis capitis, psoriasis guttata, psoriasis inversa; neurodermatitis; ichthyosis; alopecia areata; alopecia totalis; alopecia subtotalis; alopecia universalis; alopecia diffusa; atopic dermatitis; lupus erythematodes of the skin; dermatomyositis; atopic eczema; morphea; scleroderma; alopecia areata Ophiasis type; androgenic alopecia; allergic dermatitis; irritative contact dermatitis; contact dermatitis; pemphigus vulgaris; pemphigus foliaceus; pemphigus vegetans; scarring mucous membrane pemphigoid; bullous pemphigoid; mucous membrane pemphigoid; dermatitis; dermatitis herpetiformis Duhring; urticaria; necrobiosis lipoidica; erythema nodosum; prurigo simplex; prurigo nodularis; prurigo acuta; linear IgA dermatosis; polymorphic light dermatosis; erythema solaris; exanthema of the skin; drug exanthema; purpura chronica progressiva; dihydrotic eczema; eczema; fixed drug exanthema; photoallergic skin reaction; and periorale dermatitis.

24. The method of claim 22, wherein the condition, disorder or disease is a hyperproliferative disease which is selected from the group consisting of a tumor or cancer disease, precancerosis, dysplasia, histiocytosis, a vascular proliferative disease and a virus-induced proliferative disease.

25. The method of claim 24, wherein the condition, disorder or disease is a tumor or cancer disease which is selected from the group consisting of diffuse large B-cell lymphoma (DLBCL), T-cell lymphomas or leukemias, e.g., cutaneous T-cell lymphoma (CTCL), noncutaneous peripheral T-cell lymphoma, lymphoma associated with human T-cell lymphotrophic virus (HTLV), adult T-cell leukemia/lymphoma (ATLL), as well as acute lymphocytic leukemia, acute nonlymphocytic leukemia, acute myeloid leukemia, chronic lymphocytic leukemia, chronic myelogenous leukemia, Hodgkin's disease, non-Hodgkin's lymphoma, myeloma, multiple myeloma, mesothelioma, childhood solid tumors, glioma, bone cancer and soft-tissue sarcomas, common solid tumors of adults such as head and neck cancers (e.g., oral, laryngeal and esophageal), genitourinary cancers (e.g., prostate, bladder, renal, uterine, ovarian, testicular, rectal, and colon), lung cancer (e.g., small cell carcinoma and non-small cell lung carcinoma, including squamous cell carcinoma and adenocarcinoma), breast cancer, pancreatic cancer, melanoma and other skin cancers, basal cell carcinoma, metastatic skin carcinoma, squamous cell carcinoma of both ulcerating and papillary type, stomach cancer, brain cancer, liver cancer, adrenal cancer, kidney cancer, thyroid cancer, medullary carcinoma, osteosarcoma, soft-tissue sarcoma, Ewing's sarcoma, veticulum cell sarcoma, and Kaposi's sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, leiomyosarcoma, rhabdomyosarcoma, squamous cell carcinoma, adenocarcinoma, sweat gland carcinoma, sebaceous gland carcinoma, papillary carcinoma, glioblastoma, papillary adenocarcinomas, cystadenocarcinoma, bronchogenic carcinoma, seminoma, embryonal carcinoma, Wilms' tumor, small cell lung carcinoma, epithelial carcinoma, astrocytoma, medulloblastoma, craniopharyngioma, ependymoma, pinealoma, hemangioblastoma, acoustic neuroma, oligodendroglioma, meningioma, neuroblastoma, retinoblastoma, glaucoma, hemangioma, heavy chain disease and metastases.


SEQUENCE LISTING (6 sequences)

SEQ ID NO: 1 - DNA, 25 bp, Artificial Sequence
  Left HA-rev primer
tcaccatttggacgagcaggctgga

SEQ ID NO: 2 - DNA, 35 bp, Artificial Sequence
  Left HA-for primer
gacggccagtgaattcttccccagcctagttcacg

SEQ ID NO: 3 - DNA, 35 bp, Artificial Sequence
  Right HA-rev primer
cgactctagaggatcccagtggcagagcccatttc

SEQ ID NO: 4 - DNA, 24 bp, Artificial Sequence
  Right HA-for primer
tccccgcggccaaggccgagatgc

SEQ ID NO: 5 - DNA, 28 bp, Artificial Sequence
  Reporter-for primer
tcgtccaaatggtgagcaagggcgagga

SEQ ID NO: 6 - DNA, 28 bp, Artificial Sequence
  Reporter-rev primer
ccttggccgcggggaggcggcccaaagg